(19) United States
(12) Patent Application Publication
(10) Pub. No.: US 2021/0332102 A1
(43) Pub. Date: Oct. 28, 2021

(54) HIGH-AFFINITY T CELL RECEPTOR AGAINST PRAME

(71) Applicant: GUANGDONG XIANGXUE LIFE SCIENCES, LTD., Guangzhou, Guangdong (CN)

(72) Inventors: Yi LI, Guangzhou, Guangdong (CN); Jinhua HUANG, Guangzhou, Guangdong (CN); Kai ZHAN, Guangzhou, Guangdong (CN)

(21) Appl. No.: 17/254,432

(22) PCT Filed: Nov. 23, 2018
(86) PCT No.: PCT/CN2018/117238

§ 371 Date: Dec. 21, 2020

(30) Foreign Application Priority Data
Dec. 6, 2017 (CN) 201711278663.X

(51) Int. Cl. C07K 14/725; A61K 35/17

(52) U.S. Cl. CPC C07K 14/7051; A61K 38/00; A61K 35/17

(57) ABSTRACT

Provided is a T cell receptor (TCR) having a feature of binding to a VLDGLDVLL-HLA A2 complex. The binding affinity of the TCR to the VLDGLDVLL-HLA-A0201 complex is at least 2 times of the binding affinity of a wild-type TCR to the VLDGLDVLL-HLA-A0201 complex. Also provided is a fusion molecule of the TCR and a therapeutic agent. The TCR can be used alone or in combination with a therapeutic agent to target a tumor cell presenting the VLDGLDVLL-HLA-A0201 complex.

### TECHNICAL FIELD

The present invention relates to the field of biotechnology, and in particular to a T cell receptor (TCR) capable of recognizing a polypeptide derived from PRAME protein. The invention also relates to the preparation and uses of such receptors.

### BACKGROUND

There are only two types of molecules that can recognize antigens in a specific manner. One is immunoglobulin or antibody and the other is T cell receptor (TCR), which is α/β or γ/δ heterodimeric glycoprotein on cell membrane. The physical repertoire of TCR of immune system is generated in thymus through V(D)J recombination, followed by positive and negative selections. In peripheral environment, TCRs mediate the recognition of specific Major Histocompatibility Complex-peptide complexes (pMHC) by T cells and, as such, are essential to the immunological functioning of cells in the immune system.

TCR is the only receptor for presenting specific peptide antigens in Major Histocompatibility Complex (MHC). The exogenous or endogenous peptides may be the only sign of abnormality in a cell. In the immune system, once antigen-specific TCRs bind with pMHC complexes, it causes direct physical contact of a T-cell and an antigen presenting cell (APC). Then, the interaction of other membrane molecules in T cell and APC occurs and the subsequent cell signaling and other physiological responses are initiated so that a range of different antigen-specific T cells exert immune effects on their target cells.

Molecular ligands of MHC class I and class II corresponding to TCR are also proteins of the immunoglobulin superfamily but are specific for antigen presentation, and different individuals have different MHCs, thereby presenting different short peptides in one protein antigen to the surface of respective APC cells. Human MHC is commonly referred to as HLA gene or HLA complex.

PRAME is a melanoma-specific antigen (preferentially expressed antigen of melanoma, PRAME), which is expressed in 88% of primary and 95% of metastatic melanomas (Ikeda H, et al. Immunity, 1997, 6(2):199-208), but not expressed in normal skin tissues and benign melanocytes. PRAME is degraded into small molecule polypeptides after being produced in cells, and combined with MHC (major histocompatibility complex) molecules to form a complex, which is presented to the cell surface. VLDGLDVLL (SEQ ID NO: 103) is a short peptide derived from PRAME antigen and a target for treating PRAME-related diseases. In addition to melanoma, PRAME is also expressed in a variety of tumors, including lung squamous cell carcinoma, breast cancer, renal cell carcinoma, head and neck tumors, Hodgkin's lymphoma, sarcoma, medulloblastoma and the like (van't Veer LJ, et al. Nature, 2002, 415(6871): 530-536; Boon K, et al. Oncogene, 2003, 22 (48): 7687-7694). In addition, PRAME is also significantly expressed in leukemia, wherein there is 17% to 42% in acute lymphocytic leukemia, and 30% to 64% in acute myeloid leukemia (SteinbachD, et al. Cancer Genet Cytogene, 2002, 138(1): 89-91). Therefore, VLDGLDVLL-HLA A2 complex provides a marker for TCR targeting tumor cells. The TCR capable of binding to VLDGLDVLL-HLA A2 complex has high application value for treating tumors. For example, a TCR capable of targeting the tumor cell marker can be used to deliver a cytotoxic agent or immunostimulatory agent to target cells, or have the same transformed into T cells, such that the T cell expressing the TCR can destroy the tumor cells and can be administered to a patient during the course of treatment of so called adoptive immunotherapy. For the former purpose, the ideal TCR has a higher affinity, allowing the TCR to reside on the targeted cells for a long period of time. For the latter purpose, it is preferred to use a medium affinity TCR. Accordingly, a skilled person in the art is directed to developing TCRs that can be used to target tumor cell markers for different purposes.

### SUMMARY OF THE INVENTION

It is an object of the present invention to provide a TCR having a higher affinity for VLDGLDVLL-HLA-A0201 complex.

It is still another object of the present invention to provide a method for preparing the TCR of the above type and the use of the TCR of the above type.

In a first aspect of the invention, a T cell receptor (TCR) is provided, which has an activity of binding to VLDGLDVLL-HLA-A0201 complex.

In another preferred embodiment, the T cell receptor (TCR) has an activity of binding to VLDGLDVLL-HLA-A0201 complex, and the T cell receptor comprises a TCR α chain variable domain and a TCR β chain variable domain, the TCRα chain variable domain comprises three CDR regions, and the reference sequences of the three CDR regions of the TCR α chain variable domain are listed as follows,

CDR1α: DRGSQS

CDR2α: IYSNGD

CDR3α: AVARTYTGNQFY, and contains at least one of the following mutations:

| Residue before mutation | Residue after mutation |
|---|---|
| S at position 4 of CDR1α | T or A |
| S at position 6 of CDR1α | A |
| I at position 1 of CDR2α | Q or L or T |
| Y at position 2 of CDR2α | V |
| S at position 3 of CDR2α | M or V or Q |
| N at position 4 of CDR2α | P or D |
| A at position 3 of CDR3α | V |
| R at position 4 of CDR3α | L |
| T at position 5 of CDR3α | S |
| Y at position 6 of CDR3α | W |
| T at position 7 of CDR3α | K or A or R or L or Q or F |
| G at position 8 of CDR3α | S |
| N at position 9 of CDR3α | T |
| Q at position 10 of CDR3α | G or R |

and/or, the TCR β chain variable domain comprises three CDR regions, and the reference sequences of the three CDR regions of the TCR β chain variable domain are listed as follows,

CDR1β: SEHNR CDR2β: FQNEAQ CDR3β: ASSQKFSGIQPQH, and contains at least one of the following mutations:

| Residue before mutation | Residue after mutation |
|---|---|
| N at position 3 of CDR2β | D or G |
| E at position 4 of CDR2β | S or R |
| A at position 5 of CDR2β | I or S |
| Q at position 6 of CDR2β | E |
| S at position 3 of CDR3β | N |
| S at position 4 of CDR3β | A or P or N or K or Q or T or M or R |
| Q at position 5 of CDR3β | S or G or T |
| K at position 6 of CDR3β | P or G or L |
| F at position 7 of CDR3β | V or L. |

In another preferred embodiment, the number of mutations in the CDR region of the TCR α chain may be 1, 2, 3, 4, 5, 6, 7, 8, 9, 10, 11 or 12.

In another preferred embodiment, the number of mutations in the CDR region of the TCR β chain may be 1, 2, 3, 4, 5, 6, 7 or 8.

In another preferred embodiment, the T cell receptor (TCR) according to the invention comprises a TCR α chain variable domain and a TCR β chain variable domain, and said TCR α chain variable domain comprises CDR1α, CDR2α and CDR3α.

In another preferred embodiment, the CDR3α comprises a sequence:

AVAR[3αX1][3αX2][3αX3]S[3αX4][3αX5]FY, wherein [3αX1], [3αX2], [3αX3], [3αX4], [3αX5] are independently selected from any natural amino acid residue.

In another preferred embodiment, [3αX1] is T or S.

In another preferred embodiment, [3αX2] is Y or W.

In another preferred embodiment, [3αX3] is K or A or R or L or Q or F.

In another preferred embodiment, [3αX4] is T or N.

In another preferred embodiment, [3αX5] is G or R or Q.

In another preferred embodiment, [3αX1] is T or S, [3αX2] is W, [3αX3] is K, [3αX4] is T and [3αX5] is G or Q.

In another preferred embodiment, the CDR3α comprises a sequence selected from the group consisting of:

AVARTYTGNQFY,
AVARSWKSNQFY,
AVARSWASNQFY,
AVARTYRSTGFY
and
AVARTYKSTGFY.

In another preferred embodiment, the CDR1α comprises a sequence:

DRG[1αX1][1αX2][1αX3], wherein [1αX1], [1αX2] and [1αX3] are independently selected from any natural amino acid residue.

In another preferred embodiment, [1αX1] is S or T or A.

In another preferred embodiment, [1αX2] is S or Q.

In another preferred embodiment, [1αX3] is S or A.

In another preferred embodiment, [1αX1] is T or A, [1αX2] is Q and [1αX3] is A.

In another preferred embodiment, the CDR1α comprises a sequence selected from the group consisting of:

DRGSQS, DRGTQA, DRGAQA and DRGSQA.

In another preferred embodiment, the CDR2α comprises a sequence:

[2αX1][2αX2][2αX3][2αX4]GD, wherein [2αX1], [2αX2], [2αX3], [2αX4] are independently selected from any natural amino acid residue.

In another preferred embodiment, [2αX1] is I or Q or T.

In another preferred embodiment, [2αX2] is Y or V.

In another preferred embodiment, [2αX3] is S or M or V.

In another preferred embodiment, [2αX4] is N, P or D.

In another preferred embodiment, CDR2α comprises a sequence selected from the group consisting of:

IYSNGD, QVMPGD, QVVPGD and LVQPGD.

In another preferred embodiment, the TCR comprises a TCRα chain variable domain and a TCRβ chain variable domain, and the TCRβ chain comprises CDR1β, CDR2β and CDR3β, wherein the CDR1β comprises the sequence: SEHNR.

In another preferred embodiment, CDR2β comprises a sequence:

FQ[2βX1][2βX2][2βX3][2βX4], wherein each of [2βX1], [2βX2], [2βX3] and [2βX4] is independently selected from any natural amino acid residue.

In another preferred embodiment, [2βX1] is D or G.

In another preferred embodiment, [2βX2] is S or R or E.

In another preferred embodiment, [2βX3] is I or S.

In another preferred embodiment, [2βX4] is E.

In another preferred embodiment, CDR2β comprises a sequence selected from the group consisting of:

FQNEAQ, FQDSIE and FQGRSQ.

In another preferred embodiment, the CDR3β comprises a sequence: AS[3βX1][3βX2][3βX3][3βX4][3βX5]SGIQPQH, wherein [3βX1], [3βX2], [3βX3], [3βX4], [3βX5] are independently selected from any natural amino acid residue.

In another preferred embodiment, [3βX1] is S or N.

In another preferred embodiment, [3βX2] is M, R, Q, A, P, N, K, T or S.

In another preferred embodiment, [3βX3] is G, S, T or Q.

In another preferred embodiment, [3βX4] is G, P or K.

In another preferred embodiment, [3βX5] is V or F.

In another preferred embodiment, [3βX1] is N, [3βX2] is S or Q or R, [3βX3] is G or S, [3βX4] is G and [3βX5] is F.

In another preferred embodiment, the CDR3β comprises a sequence selected from the group consisting of:

ASSSQKFSGIQPQH,
ASNSGPVSGIQPQH,
ASNQSGFSGIQPQH,
ASSMSGFSGIQPQH
and
ASSSGLLSGIQPQH.

In another preferred embodiment, the TCRα chain variable domain of the TCR does not simultaneously comprise the following CDRs:

CDR1α: DRGSQS; CDR2α: IYSNGD; and CDR3α: AVARTYTGNQFY.

In another preferred embodiment, the TCRβ chain variable domain of the TCR does not simultaneously comprise the following CDRs:

CDR1β: SEHNR; CDR2β: FQNEAQ: and CDR3β: ASSSQKFSGIQPQH.

In another preferred embodiment, the mutation occurs in one or more CDR regions of the α chain and/or β chain variable domain.

In another preferred embodiment, the mutation occurs in CDR1, CDR2 and/or CDR3 of the α chain, and/or the mutation occurs in CDR2 and/or CDR3 of the β chain.

In a preferred embodiment of the invention, the affinity of the TCR for VLDGLDVLL-HLA-A0201 complex is at least 2 times of that of the wild type TCR; preferably at least 5 times; more preferably at least 10 times.

In another preferred embodiment, the affinity of the TCR for VLDGLDVLL-HLA-A0201 complex is at least 50 times of that of the wild type TCR; preferably, at least 100 times; more preferably, at least 500 times; most preferably, at least 1000 times.

In another preferred embodiment, the affinity of the TCR for VLDGLDVLL-HLA-A0201 complex is at least 10⁴ times of that of the wild type TCR; preferably, at least 10⁵ times; more preferably, at least 10⁶ times; most preferably, at least 10⁷ times.

In particular, the dissociation equilibrium constant K_{D} of the TCR to VLDGLDVLL-HLA-A0201 complex is ≤5 μM;

In another preferred embodiment, the dissociation equilibrium constant of the TCR to VLDGLDVLL-HLA-A0201 complex is 10 nM≤K_{D}≤50 nM; preferably, 50 nM≤K_{D}≤500 nM; more preferably, 100 nM≤K_{D}≤500 nM;

In another preferred embodiment, the dissociation equilibrium constant of the TCR to VLDGLDVLL-HLA-A0201 complex is 50 pM≤K_{D}≤500 pM; preferably, 50 pM≤K_{D}≤100 pM.

In another preferred embodiment, the TCR has CDRs selected from the group consisting of:

| TCR No. | CDR1α | CDR2α | CDR3α | CDR1β | CDR2β | CDR3β |
|---|---|---|---|---|---|---|
| 1 | DRGSQS | IYSNGD | AVARSWKSNQFY | SEHNR | FQNEAQ | ASSSQKFSGIQPQH |
| 2 | DRGSQS | IYSNGD | AVARTYLSNRFY | SEHNR | FQNEAQ | ASSSQKFSGIQPQH |
| 3 | DRGSQS | IYSNGD | AVARTWASNQFY | SEHNR | FQNEAQ | ASSSQKFSGIQPQH |
| 4 | DRGSQS | IYSNGD | AVARSYQSNQFY | SEHNR | FQNEAQ | ASSSQKFSGIQPQH |
| 5 | DRGSQS | IYSNGD | AVARSWFSNQFY | SEHNR | FQNEAQ | ASSSQKFSGIQPQH |
| 6 | DRGSQS | IYSNGD | AVARTYKSNRFY | SEHNR | FQNEAQ | ASSSQKFSGIQPQH |
| 7 | DRGSQS | IYSNGD | AVARTYKSNQFY | SEHNR | FQNEAQ | ASSSQKFSGIQPQH |
| 8 | DRGSQS | IYSNGD | AVARTYTGNQFY | SEHNR | FQNEAQ | ASNSSGFSGIQPQH |
| 9 | DRGSQS | IYSNGD | AVARTYTGNQFY | SEHNR | FQNEAQ | ASSASGFSGIQPQH |
| 10 | DRGSQS | IYSNGD | AVARTYTGNQFY | SEHNR | FQNEAQ | ASSPSGFSGIQPQH |
| 11 | DRGSQS | IYSNGD | AVARTYTGNQFY | SEHNR | FQNEAQ | ASNNSGFSGIQPQH |
| 12 | DRGSQS | IYSNGD | AVARTYTGNQFY | SEHNR | FQNEAQ | ASNKSGFSGIQPQH |
| 13 | DRGSQS | IYSNGD | AVARTYTGNQFY | SEHNR | FQNEAQ | ASNSGPVSGIQPQH |
| 14 | DRGSQS | IYSNGD | AVARTYTGNQFY | SEHNR | FQNEAQ | ASNQSGFSGIQPQH |
| 15 | DRGSQS | IYSNGD | AVARTYTGNQFY | SEHNR | FQNEAQ | ASNTSGFSGIQPQH |
| 16 | DRGSQS | IYSNGD | AVARTYTGNQFY | SEHNR | FQNEAQ | ASSMSGFSGIQPQH |
| 17 | DRGSQS | IYSNGD | AVARTYTGNQFY | SEHNR | FQNEAQ | ASSSGLLSGIQPQH |
| 18 | DRGSQS | QVMPGD | AVARSYKSNQFY | SEHNR | FQDSIE | ASNSGPVSGIQPQH |
| 19 | DRGSQS | QVVPGD | AVARTYKSTGFY | SEHNR | FQGRSQ | ASNSGPVSGIQPQH |
| 20 | DRGTQA | QVMPGD | AVARTYKSNGFY | SEHNR | FQDSIE | ASNSSGFSGIQPQH |
| 21 | DRGSQS | QVMPGD | AVARSWASNQFY | SEHNR | FQDSIE | ASSMSGFSGIQPQH |
| 22 | DRGSQS | QVMPGD | AVARSWKSNQFY | SEHNR | FQDSIE | ASSRTGFSGIQPQH |
| 23 | DRGSQA | QVMPGD | AVARTYRSTGFY | SEHNR | FQDSIE | ASNSGPVSGIQPQH |
| 24 | DRGTQA | QVMPGD | AVARTYKSTGFY | SEHNR | FQDSIE | ASNSSGFSGIQPQH |
| 25 | DRGSQS | QVMPGD | AVARTWASNQFY | SEHNR | FQDSIE | ASNQSGFSGIQPQH |
| 26 | DRGTQA | LVQPGD | AVARTYKSTGFY | SEHNR | FQDSIE | ASNSGPVSGIQPQH |
| 27 | DRGSQA | QVVPGD | AVARTWASNQFY | SEHNR | FQDSIE | ASNSGPVSGIQPQH |
| 28 | DRGSQS | LVQPGD | AVARTYKSTGFY | SEHNR | FQGSAQ | ASNSGPVSGIQPQH |
| 29 | DRGAQA | QVMPGD | AVARSWASNQFY | SEHNR | FQDSIE | ASNQSGFSGIQPQH |
| 30 | DRGTQA | QVVPGD | AVARSWKSNQFY | SEHNR | FQDSIE | ASNQSGFSGIQPQH |
| 31 | DRGSQA | QVMPGD | AVARSWTSNQFY | SEHNR | FQDSIE | ASNQTGFSGIQPQH |
| 32 | DRGAQA | IYSNGD | AVARSYKSNQFY | SEHNR | FQDSIE | ASSRTGFSGIQPQH |
| 33 | DRGAQA | TVQDGD | AVARSWKSNQFY | SEHNR | FQDSIE | ASNQSGFSGIQPQH |
| 34 | DRGSQA | IYSNGD | AVARSWKSNQFY | SEHNR | FQDSIE | ASNSGPVSGIQPQH |
| 35 | DRGTQA | QVMPGD | AVVLSYKSNGFY | SEHNR | FQDSIE | ASNSSGFSGIQPQH |
| 36 | DRGAQA | QVMPGD | AVARSWKSNQFY | SEHNR | FQDSIE | ASSRTGFSGIQPQH |

In another preferred embodiment, the TCR is soluble.

In another preferred embodiment, the TCR is an αβ heterodimeric TCR or a single chain TCR.

In another preferred embodiment, the TCR of the present invention is an αβ heterodimeric TCR, and the α chain variable domain of the TCR comprises an amino acid sequence having at least 85%, preferably at least 90%; more preferably, at least 92%; most preferably, at least 94% (e.g., may be at least 88%, 89%, 90%, 91%, 92%, 93%, 94%, 95%, 96%, 97%, 98%, 99% of sequence homology) of sequence homology with the amino acid sequence shown in SEQ ID NO: 1; and/or the β chain variable domain of the TCR comprises an amino acid sequence having at least 90%, preferably at least 92%; more preferably, at least 94%; most preferably at least 97% (e.g., may be at least 91%, 92%, 93%, 94%, 95%, 96%, 97%, 98%, 99% of sequence homology) of sequence homology with the amino acid sequence shown in SEQ ID NO: 2.

In another preferred embodiment, the TCR comprises (i) all or part of the TCR α chain other than its transmembrane domain, and (ii) all or part of the TCR β chain other than its transmembrane domain, wherein both of (i) and (ii) comprise the variable domain and at least a portion of the constant domain of the TCR chain.

In another preferred embodiment, the TCR is an αβ heterodimeric TCR, and an artificial interchain disulfide bond is contained between the α chain variable region and the β chain constant region of the TCR.

In another preferred embodiment, cysteine residues forming an artificial interchain disulfide bond between the α chain variable region and the β chain constant region of the TCR are substituted for one or more groups of amino acids selected from the following:

amino acid at position 46 of TRAV and amino acid at position 60 of TRBC1*01 or TRBC2*01 exon 1;

amino acid at position 47 of TRAV and amino acid at position 61 of TRBC1*01 or TRBC2*01 exon 1;

amino acid at position 46 of TRAV and amino acid at position 61 of TRBC1*01 or TRBC2*01 exon 1; or

amino acid at position 47 of TRAV and amino acid at position 60 of TRBC1*01 or TRBC2*01 exon 1;

wherein the position number in an amino acid sequence is based on the position number listed in IMGT (International Immunogenetics Information System).

In another preferred embodiment, the TCR comprising an artificial interchain disulfide bond between α chain variable region and β chain constant region comprises α chain variable domain and β chain variable domain as well as all or part of β chain constant domains other than its transmembrane domain, however it does not comprise α chain constant domain, and α chain variable domain and β chain of the TCR form a heterodimer.

In another preferred embodiment, the TCR comprising an artificial interchain disulfide bond between α chain variable region and β chain constant region comprises (i) all or part of TCR α chain other than its transmembrane domain, and (ii) all or part of TCR β chain other than its transmembrane domain, wherein both of (i) and (ii) comprise the variable domain and at least a portion of constant domains of the TCR chain.

In another preferred embodiment, the TCR is an αβ heterodimeric TCR comprising (i) all or part of TCR α chain other than its transmembrane domain, and (ii) all or part of TCR β chain other than its transmembrane domain, wherein both of (i) and (ii) comprise the variable domain and at least a portion of the constant domain of the TCR chain, and an artificial interchain disulfide bond is contained between α chain constant region and β chain constant region.

In another preferred embodiment, cysteine residues forming an artificial interchain disulfide bond between the TCR α chain constant region and β chain constant region are substituted for one or more groups of amino acids selected from the following:

Thr48 of TRAC*01 exon 1 and Ser57 of TRBC1*01 or TRBC2*01 exon 1;

Thr45 of TRAC*01 exon 1 and Ser77 of TRBC1*01 or TRBC2*01 exon 1;

Tyr10 of TRAC*01 exon 1 and Ser17 of TRBC1*01 or TRBC2*01 exon 1;

Thr45 of TRAC*01 exon 1 and Asp59 of TRBC1*01 or TRBC2*01 exon 1;

Ser15 of TRAC*01 exon 1 and Glu15 of TRBC1*01 or TRBC2*01 exon 1;

Arg53 of TRAC*01 exon 1 and Ser54 of TRBC1*01 or TRBC2*01 exon 1;

Pro89 of TRAC*01 exon 1 and Ala19 of TRBC1*01 or TRBC2*01 exon 1; and

Tyr10 of TRAC*01 exon 1 and Glu20 of TRBC1*01 or TRBC2*01 exon 1;

wherein the position number in an amino acid sequence is based on the position number listed in IMGT (International Immunogenetics Information System).

In another preferred embodiment, the TCR is a single chain TCR.

In another preferred embodiment, the TCR is a single-chain TCR consisting of an α chain variable domain and a β chain variable domain, and the α chain variable domain and the β chain variable domain are connected by a flexible short peptide sequence (linker).

In another preferred embodiment, the hydrophobic core of the TCR α chain variable domain and/or β chain variable domain is mutated.

In another preferred embodiment, the TCR, in which the hydrophobic core is mutated, is a single-chain TCR consisting of an α variable domain and a β variable domain, and the α variable domain and the β variable domain are connected by a flexible short peptide sequence (linker).

In another preferred embodiment, the TCR of the present invention is a single-chain TCR, and the α chain variable domain of the TCR comprises an amino acid sequence having at least 85%, preferably at least 90%; more preferably, at least 92%; most preferably at least 94% (e.g., may be at least 88%, 89%, 90%, 91%, 92%, 93%, 94%, 95%, 96%, 97%, 98%, 99% of sequence homology) of sequence homology with the amino acid sequence shown in SEQ ID NO: 3; and/or the β chain variable domain of the TCR comprises an amino acid sequence having at least 90%, preferably at least 92%; more preferably, at least 94%; most preferably at least 97% (e.g., may be at least 91%, 92%, 93%, 94%, 95%, 96%, 97%, 98%, 99% of sequence homology) of sequence homology with the amino acid sequence shown in SEQ ID NO: 4.

In another preferred embodiment, the amino acid sequence of the α chain variable domain of the TCR is selected from the group consisting of: SEQ ID NOs: 9-34 and 57-82; and/or the amino acid sequence of the β chain variable domain of the TCR is selected from the group consisting of: SEQ ID NOs: 35-52 and 83-100.

In another preferred embodiment the TCR is selected from the ground consisting of:

| | Sequence of α chain variable | Sequence of β chain variable |
|---|---|---|
| TCR No. | domain SEQ ID NO: | domain SEQ ID NO: |
| s-1 | 9 | 4 |
| s-2 | 10 | 4 |
| s-3 | 11 | 4 |
| s-4 | 12 | 4 |
| s-5 | 13 | 4 |
| s-6 | 14 | 4 |
| s-7 | 15 | 4 |
| s-8 | 3 | 35 |
| s-9 | 3 | 36 |
| s-10 | 3 | 37 |
| s-11 | 3 | 38 |
| s-12 | 3 | 39 |
| s-13 | 3 | 40 |
| s-14 | 3 | 41 |
| s-15 | 3 | 42 |
| s-16 | 3 | 43 |
| s-17 | 3 | 44 |
| s-18 | 16 | 45 |
| s-19 | 17 | 46 |
| s-20 | 18 | 47 |
| s-21 | 19 | 48 |
| s-22 | 20 | 49 |
| s-23 | 21 | 45 |
| s-24 | 22 | 47 |
| s-25 | 23 | 50 |
| s-26 | 24 | 45 |
| s-27 | 25 | 45 |
| s-28 | 26 | 51 |
| s-29 | 27 | 50 |
| s-30 | 28 | 50 |
| s-31 | 29 | 52 |
| s-32 | 30 | 49 |
| s-33 | 31 | 50 |
| s-34 | 32 | 45 |
| s-35 | 33 | 47 |
| s-36 | 34 | 49. |

In another preferred embodiment, the TCR is selected from the group consisting of:

| | Sequence of α chain variable | Sequence of β chain variable |
|---|---|---|
| TCR No. | domain SEQ ID NO: | domain SEQ ID NO: |
| 1 | 57 | 2 |
| 2 | 58 | 2 |
| 3 | 59 | 2 |
| 4 | 60 | 2 |
| 5 | 61 | 2 |
| 6 | 62 | 2 |
| 7 | 63 | 2 |
| 8 | 1 | 83 |
| 9 | 1 | 84 |
| 10 | 1 | 85 |
| 11 | 1 | 86 |
| 12 | 1 | 87 |
| 13 | 1 | 88 |
| 14 | 1 | 89 |
| 15 | 1 | 90 |
| 16 | 1 | 91 |
| 17 | 1 | 92 |
| 18 | 64 | 93 |
| 19 | 65 | 94 |
| 20 | 66 | 95 |
| 21 | 67 | 96 |
| 22 | 68 | 97 |
| 23 | 69 | 93 |
| 24 | 70 | 95 |
| 25 | 71 | 98 |
| 26 | 72 | 93 |
| 27 | 73 | 93 |
| 28 | 74 | 99 |
| 29 | 75 | 98 |
| 30 | 76 | 98 |
| 31 | 77 | 100 |
| 32 | 78 | 97 |
| 33 | 79 | 98 |
| 34 | 80 | 93 |
| 35 | 81 | 95 |
| 36 | 82 | 97 |

In another preferred embodiment, a conjugate binds to the α chain and/or β chain of the TCR at C- or N-terminal.

In another preferred embodiment, the conjugate that binds to the TCR is a detectable label, a therapeutic agent, a PK modified moiety, or a combination thereof.

In another preferred embodiment, the therapeutic agent that binds to the TCR is an anti-CD3 antibody linked to the α or β chain of the TCR at C- or N-terminal.

In a preferred embodiment of the invention, the T cell receptor (TCR) has the activity of binding to VLDGLDVLL-HLA-A0201 complex and comprises a TCR α chain variable domain and a TCR β chain variable domain, there is a mutation in the α chain variable domain of the TCR as shown in SEQ ID NO: 1, and the mutated amino acid residue site includes one or more of 30S, 32S, 50I, 51Y, 52S, 53N, 92A, 93R, 94T, 95Y, 96T, 97G, 98N and 99Q, wherein the amino acid residue is numbered as shown in SEQ ID NO: 55 or 1; and/or there is a mutation in the β chain variable domain of the TCR as shown in SEQ ID NO: 2, and the mutated amino acid residue site includes one or more of 51N, 52E, 53A, 54Q, 95S, 96S, 97Q, 98K and 99F, wherein the amino acid residue is numbered as shown in SEQ ID NO: 56 or 2;

preferably, upon mutation, the TCR α chain variable domain comprises one or more amino acid residues selected from the group consisting of: 30T or 30A; 32A; 50Q, SOL or 50T; 51V; 52M, 52V, 52Q; 53P or 53D; 92V; 93L; 94S; 95W; 96K, 96A, 96R, 96L, 96Q, 96F or 97S; 98T; and 99R or 99G, wherein the amino acid residue is numbered as shown in SEQ ID NO: 1; and/or upon mutation, the TCR β chain variable domain comprises one or more amino acid residues selected from the group consisting of: 51D or 51G; 52S or 52R; 53I or 53S; 54E; 95N; 96A, 96P, 96N, 96K, 96Q, 96T, 96M or 96R;975, 97G or 97T; 98G, 98P or 98L; 99V or 99L; wherein the amino acid residue is numbered as shown in SEQ ID NO: 2.

In a second aspect of the invention, a multivalent TCR complex comprising at least two TCR molecules is provided, and at least one TCR molecule is the TCR of the first aspect of the invention.

In a third aspect of the invention, a nucleic acid molecule is provided, comprising a nucleic acid sequence encoding the TCR molecule of the first aspect of the invention or the multivalent TCR complex of the second aspect of the invention, or a complement sequence thereof.

In a fourth aspect of the invention, a vector is provided, comprising the nucleic acid molecule of the third aspect of the invention.

In a fifth aspect of the present invention, a host cell is provided, comprising the vector of the fourth aspect of the present invention or having the exogenous nucleic acid molecule of the third aspect of the present invention integrated into its genome.

In a sixth aspect of the invention, an isolated cell is provided, expressing the TCR of the first aspect of the invention.

In a seventh aspect of the invention, a pharmaceutical composition is provided, comprising a pharmaceutically acceptable carrier, and a TCR of the first aspect of the invention, or a TCR complex of the second aspect of the invention, or the cell of the sixth aspect of the invention.

In an eighth aspect of the present invention, a method for treating a disease is provided, comprising administering an appropriate amount of the TCR of the first aspect of the present invention, or the TCR complex of the second aspect of the present invention, or the cell of the sixth aspect of the invention, or the pharmaceutical composition of the seventh aspect of the invention to a subject in need thereof.

In a ninth aspect of the invention, use of the TCR of the first aspect of the invention, or the TCR complex of the second aspect of the invention, or the cell of the sixth aspect of the invention is provided for preparing a medicament for treating tumor.

In a tenth aspect of the invention, a method for preparing the T cell receptor of the first aspect of the invention is provided, comprising the steps of:

(i) culturing the host cell of the fifth aspect of the invention to express the T cell receptor of the first aspect of the invention;

(ii) isolating or purifying the T cell receptor.

It is to be understood that within the scope of the present invention, the various technical features of the present invention and the technical features specifically described hereinafter (as in the embodiments) may be combined with each other to constitute a new or preferred technical solution, which will not be repeated herein one by one.

### DESCRIPTION OF DRAWINGS

FIG. 1a and FIG. 1b show the amino acid sequences of wild-type TCR α and β chain variable domain that are capable of specifically binding to VLDGLDVLL-HLA-A0201 complex, respectively.

FIG. 2a and FIG. 2b show the amino acid sequences of the α variable domain and the β chain variable domain of the single-chain template TCR constructed in the present invention, respectively.

FIG. 3a and FIG. 3b show the DNA sequences of the α variable domain and the β chain variable domain of the single-chain template TCR constructed in the present invention, respectively.

FIG. 4a and FIG. 4b are the amino acid sequence and nucleotide sequence of the linking short peptide (linker) of the single-chain template TCR constructed in the present invention, respectively.

FIGS. 5(1)-(26) show the amino acid sequences of α-chain variable domain of single-chain TCRs with high affinity for VLDGLDVLL-HLA-A0201 complex, respectively, and the mutated residues are underlined.

FIGS. 6(1)-(18) show the amino acid sequences of β-chain variable domain of single-chain TCRs with high affinity for VLDGLDVLL-HLA-A0201 complex, respectively, and the mutated residues are underlined.

FIG. 7a and FIG. 7b show the amino acid sequence and DNA sequence of the single-chain template TCR constructed in the present invention, respectively.

FIGS. 8a and 8b show the amino acid sequences of the reference TCR α and β chains in the present invention, respectively.

FIGS. 9(1)-(26) show the amino acid sequences of α-chain variable domain of a heterodimeric TCR with high affinity for VLDGLDVLL-HLA-A0201 complex, respectively, and the mutated residues are underlined.

FIGS. 10(1)-(18) show the amino acid sequences of β-chain variable domain of a heterodimeric TCR with high affinity for VLDGLDVLL-HLA-A0201 complex, respectively, and the mutated residues are underlined.

FIG. 11a and FIG. 11b show the amino acid sequences of wild-type TCR α and β chain that are capable of specifically binding to VLDGLDVLL-HLA-A0201 complex, respectively.

FIG. 12 is a binding curve of a wild-type TCR with VLDGLDVLL-HLA-A0201 complex.

FIGS. 13a and 13b show results of INF-γ activation experiment of effector cells transfected with the high affinity TCR of the present invention.

FIGS. 14a-f show results of redirection experiments of a fusion protein formed by the high affinity TCR of the present invention and anti-CD3 antibody on effector cells.

### MODES FOR CARRYING OUT THE INVENTION

Through extensive and intensive research, the present inventors obtained a high affinity T cell receptor (TCR) recognizing VLDGLDVLL short peptide (derived from PRAME protein) which is presented in a form of peptide-HLA-A0201 complexe. The high affinity TCR has a mutation in three CDR regions of its α chain variable domain

CDR1α:
DRGSQS
CDR2α:
IYSNGD
CDR3α:
AVARTYTGNQFY;
and/or has a mutation in three CDR regions of
its β chain variable domain
CDR1β:
SEHNR
CDR2β:
FQNEAQ

CDR3β: ASSQKFSGIQPQH; and, after mutation, the affinity and/or binding half-life of the TCR of the present invention for the above VLDGLDVLL-HLA-A0201 complex is at least 2-fold greater than that of the wild-type TCR.

Before the present invention is described, it is to be understood that the invention is not limited to the specific methods and experimental conditions described, as such methods and conditions may vary. It is also understood that the terminology used herein is for the purpose of describing particular embodiments, and is not intended to be limiting, and the scope of the present invention shall be only limited by the attached claim set.

All technical and scientific terms used herein have the same meaning as commonly understood by a skilled person in the art to which this invention belongs, unless otherwise defined.

Although any methods and materials similar or equivalent to those described in the present invention can be used in the practice or testing of the present invention, the preferred methods and materials are exemplified herein.

Term

T Cell Receptor (TCR)

International Immunogenetics Information System (IMGT) can be used to describe a TCR. A native αβ heterodimeric TCR has an α chain and a β chain. Generally speaking, each chain comprises a variable region, a junction region and a constant region, and the β chain typically also contains a short hypervariable region between the variable region and junction region, which however is often considered as a part of the junction region. The TCR junction region is determined by the unique TRAJ and TRBJ of IMGT, and the constant region of a TCR is determined by TACT and TRBC of IMGT.

Each variable region comprises three CDRs (complementarity determining regions), CDR1, CDR2 and CDR3, which are chimeric in the framework sequence. In IMGT nomenclature, the different numbers of TRAV and TRBV refer to different Vα types and Vβ types, respectively. In IMGT system, there are following symbols for α chain constant domain: TRAC*01, where “TR” represents T cell receptor gene; “A” represents α chain gene; C represents the constant region; “*01” represents allele gene 1. There are following symbols for β-chain constant domain: TRBC1*01 or TRBC2*01, where “TR” represents T cell receptor gene; “B” represents β-chain gene; C represents constant region; “*01” represents allele gene 1. The constant region of α chain is uniquely defined, and in the form of β chain, there are two possible constant region genes “C1” and “C2”. A skilled person in the art can obtain constant region gene sequences of TCR α and β chains through the disclosed IMGT database.

The α and β chains of TCR are generally considered as having two “domains” respectively, i.e., variable domain and constant domain. The variable domain consists of a connected variable region and a connection region. Therefore, in the specification and claims of the present application, “TCR α chain variable domain” refers to a connected TRAV and TRAJ region, and likewise, “TCR β chain variable domain” refers to a connected TRBV and TRBD/TRBJ region. The three CDRs of TCR α chain variable domain are CDR1α, CDR2α and CDR3α, respectively; and the three CDRs of TCR β chain variable domain are CDR1β, CDR2β and CDR3β, respectively. The framework sequences of TCR variable domains of the invention may be of murine or human origin, preferably of human origin. The constant domain of TCR comprises an intracellular portion, transmembrane region, and extracellular portion. To obtain a soluble TCR for determining the affinity between TCR and VLDGLDVLL-HLA-A0201 complex, TCR of the invention preferably does not comprise a transmembrane region. More preferably, the amino acid sequence of the TCR of the present invention refers to the extracellular amino acid sequence of the TCR.

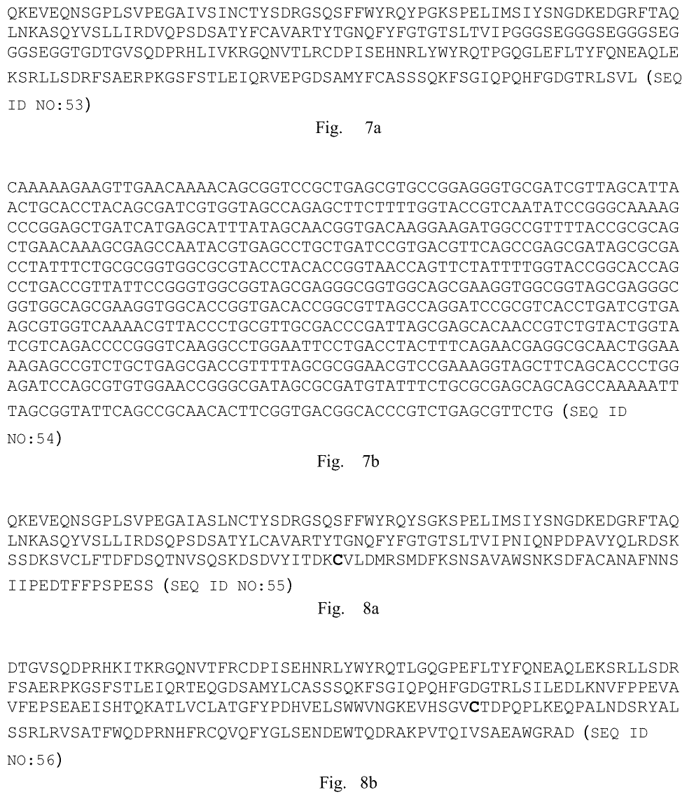

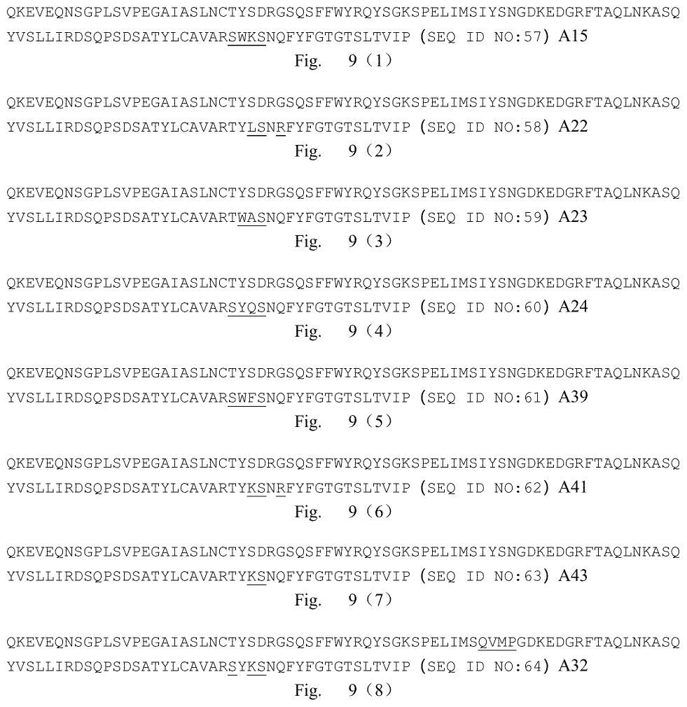

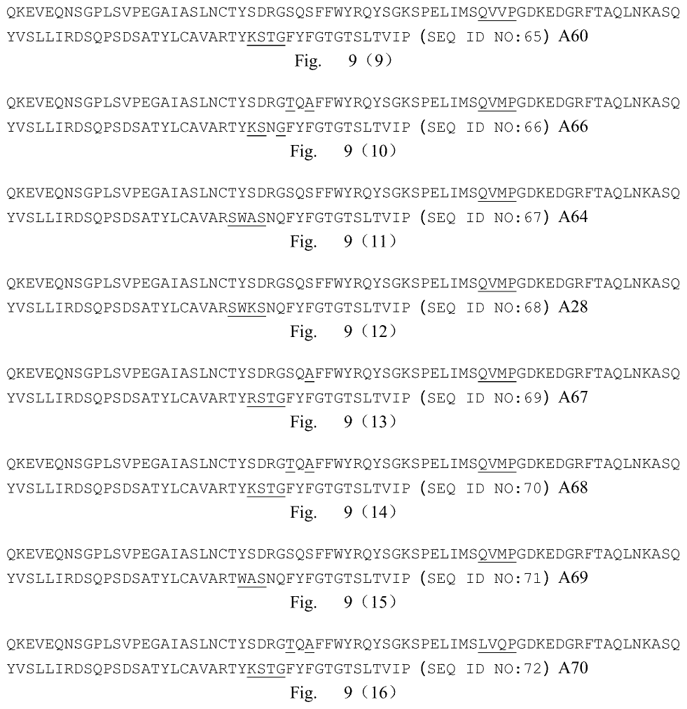

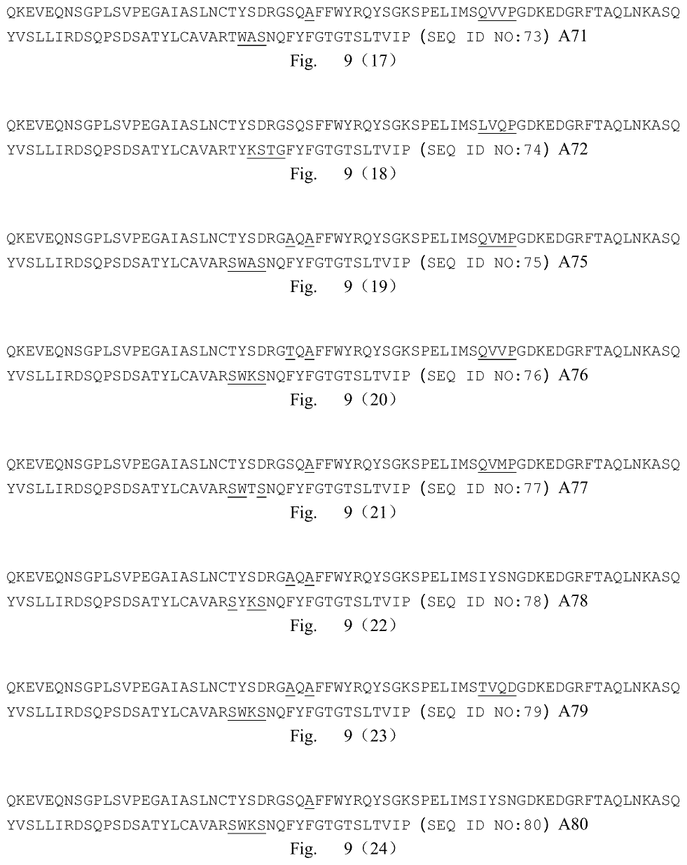

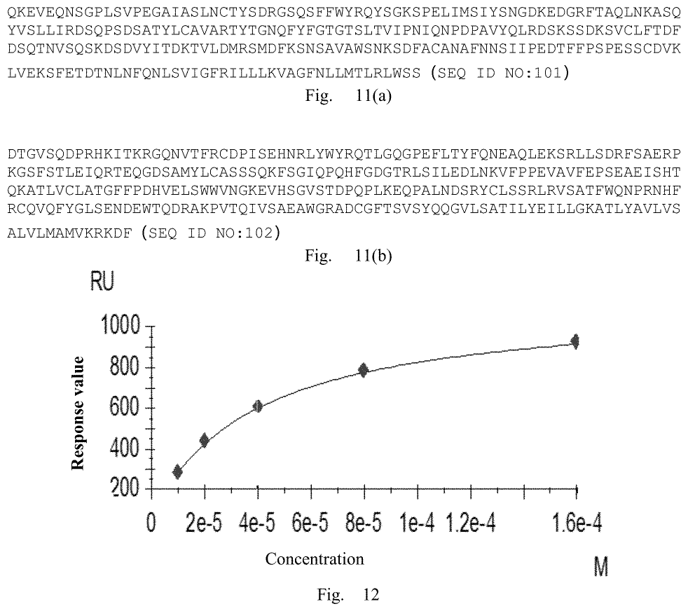

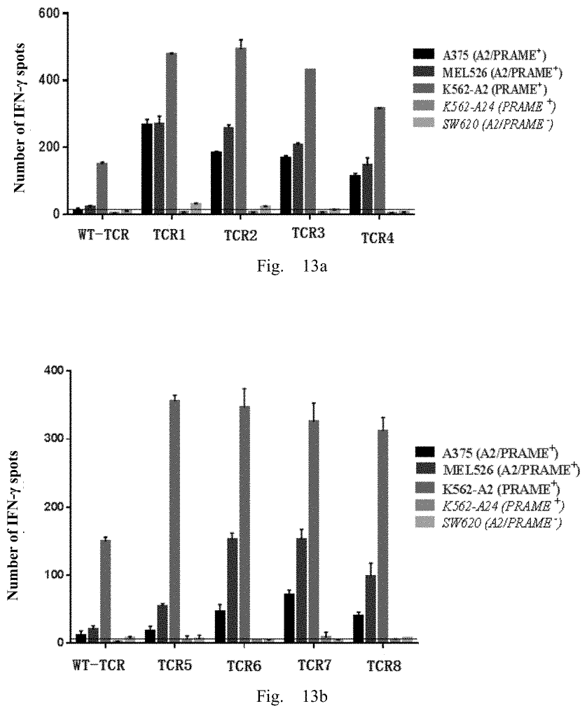

The α chain amino acid sequence and β chain amino acid sequence of the “wild type TCR” described in the present invention are SEQ ID NO: 101 and SEQ ID NO: 102, respectively, as shown in FIGS. 11a and 11b. In the present invention, the α chain amino acid sequence and β chain amino acid sequence of the “reference TCR” are SEQ ID NO: 56 and SEQ ID NO: 57, respectively, as shown in FIGS. 8a and 8b. In the present invention, the α and β chain variable domain amino acid sequences of the wild type TCR capable of binding to VLDGLDVLL-HLA-A0201 complex are SEQ ID NO: 1 and SEQ ID NO: 2, respectively, as shown in FIGS. 1a and 1b. In the present invention, the terms “polypeptide of the present invention”, “TCR of the present invention” and “T cell receptor of the present invention” are used interchangeably.

In a preferred embodiment of the invention, the T cell receptor (TCR) according to the invention comprises a TCR α chain variable domain and a TCR β chain variable domain, and said TCR α chain variable domain comprising CDR1α, CDR2α and CDR3α.

In another preferred embodiment, the CDR3α comprises a sequence:

AVAR[3αX1][3αX2][3αX3]S[3αX4][3αX5]FY, wherein [3αX1], [3αX2], [3αX3], [3αX4], [3αX5] are independently selected from any natural amino acid residues.

In another preferred embodiment, [3αX1] is T or S.

In another preferred embodiment, [3αX2] is Y or W.

In another preferred embodiment, [3αX3] is K or A or R or L or Q or F.

In another preferred embodiment, [3αX4] is T or N.

In another preferred embodiment, [3αX5] is G or R or Q.

In another preferred embodiment, [3αX1] is T or S, [3αX2] is W, [3αX3] is K, [3αX4] is T and [3αX5] is G or Q.

In another preferred embodiment, the CDR3α comprises a sequence selected from the group consisting of:

AVARTYTGNQFY,
AVARSWKSNQFY,
AVARSWASNQFY,
AVARTYRSTGFY
and
AVARTYKSTGFY.

In another preferred embodiment, the CDR1α comprises a sequence:

DRG[1αX1][1αX2][1αX3], wherein [1αX1], [1αX2] and [1αX3] are independently selected from any natural amino acid residue.

In another preferred embodiment, [1αX1] is S or T or A.

In another preferred embodiment, [1αX2] is S or Q.

In another preferred embodiment, [1αX3] is S or A.

In another preferred embodiment, [1αX1] is T or A, [1αX2] is Q and [1αX3] is A.

In another preferred embodiment, the CDR1α comprises a sequence selected from the group consisting of:

DRGSQS, DRGTQA, DRGAQA and DRGSQA.

In another preferred embodiment, the CDR2α comprises a sequence:

[2αX1][2αX2][2αX3][2αX4]GD, wherein [2αX1], [2αX2], [2αX3], [2αX4] are independently selected from any natural amino acid residue.

In another preferred embodiment, [2αX1] is I or Q or T.

In another preferred embodiment, [2αX2] is Y or V.

In another preferred embodiment, [2αX3] is S or M or V.

In another preferred embodiment, [2αX4] is N, P or D.

In another preferred embodiment, CDR2α comprises a sequence selected from the group consisting of:

IYSNGD, QVMPGD, QVVPGD and LVQPGD.

In another preferred embodiment, the TCR comprises a TCRα chain variable domain and a TCRβ chain variable domain, and the TCRβ chain comprises CDR1β, CDR2β and CDR3β, wherein the CDR1β comprises the sequence: SEHNR.

In another preferred embodiment, CDR2β comprises a sequence:

FQ[2βX1][2βX2][2βX3][2βX4], wherein each of [2βX1], [2βX2], [2βX3] and [2βX4] is independently selected from any natural amino acid residue.

In another preferred embodiment, [2βX1] is D or G.

In another preferred embodiment, [2βX2] is S or R or E.

In another preferred embodiment, [2βX3] is I or S.

In another preferred embodiment, [2βX4] is E.

In another preferred embodiment, CDR2β comprises a sequence selected from the group consisting of:

FQNEAQ, FQDSIE and FQGRSQ.

In another preferred embodiment, the CDR3β comprises a sequence:

AS[3βX1][3βX2][3βX3][3βX4][3βX5]SGIQPQH, wherein [3βX1], [3βX2], [3βX3], [3βX4], [3βX5] are independently selected from any natural amino acid residue.

In another preferred embodiment, [3βX1] is S or N.

In another preferred embodiment, [3βX2] is M, R, Q, A, P, N, K, T or S.

In another preferred embodiment, [3βX3] is G, S, T or Q.

In another preferred embodiment, [3βX4] is G, P or K.

In another preferred embodiment, [3βX5] is V or F.

In another preferred embodiment, [3βX1] is N, [3βX2] is S or Q or R, [3βX3] is G or S, [3βX4] is G and [3βX5] is F.

In another preferred embodiment, the CDR3β comprises a sequence selected from the group consisting of:

ASSSQKFSGIQPQH,
ASNSGPVSGIQPQH,
ASNQSGFSGIQPQH,
ASSMSGFSGIQPQH
and
ASSSGLLSGIQPQH.

In another preferred embodiment, the TCRα chain variable domain of the TCR does not simultaneously comprise the following CDRs:

CDR1α: DRGSQS; CDR2α: IYSNGD; and CDR3α: AVARTYTGNQFY.

In another preferred embodiment, the TCRβ chain variable domain of the TCR does not simultaneously comprise the following CDRs:

CDR1β: SEHNR; CDR2β: FQNEAQ: and CDR3β: ASSSQKFSGIQPQH.

Natural Interchain Disulfide Bond and Artificial Interchain Disulfide Bond

A group of disulfide bonds is present between the Cα and Cβ chains in the membrane proximal region of a native TCR, which is named herein as “natural interchain disulfide bond”. In the present invention, an interchain covalent disulfide bond which is artificially introduced and the position of which is different from the position of a natural interchain disulfide bond is named as “artificial interchain disulfide bond”.

For convenience of description, in the present invention, the positions of the amino acid sequences of TRAC*01 and TRBC1*01 or TRBC2*01 are sequentially numbered in order from N-terminal to C-terminal. For example, the 60^{th} amino acid in the order from N-terminal to C-terminal in TRBC1*01 or TRBC2*01 is P (valine), which can be described as Pro60 of TRBC1*01 or TRBC2*01 exon 1 in the present invention, and can also be expressed as the amino acid at position 60 of TRBC1* 01 or TRBC2*01 exon 1. For another example, the 61^{st} amino acid in the order from N-terminal to C-terminal in TRBC1*01 or TRBC2*01 is Q (glutamine), which can be described as Gln61 of TRBC1*01 or TRBC2*01 exon 1 in the invention, and can also be expressed as the amino acid at position 61 of TRBC1*01 or TRBC2*01 exon 1, and so on. In the present invention, the positions of the amino acid sequences of variable regions TRAV and TRBV are numbered according to the positions listed in IMGT. As for an amino acid in TRAV, the position is numbered as 46 in IMGT, which is described in the present invention as the amino acid at position 46 of TRAV, and so on. In the present invention, if the sequence positions of other amino acids are specifically described, the special description shall prevail.

Tumor

The term “tumor” refers to include all types of cancer cell growth or carcinogenic processes, metastatic tissues or malignant transformed cells, tissues or organs, regardless of pathological type or stage of infection. Examples of tumors include, without limitation, solid tumors, soft tissue tumors, and metastatic lesions. Examples of solid tumors include: malignant tumors of different organ systems, such as sarcoma, lung squamous cell carcinoma, and cancer. For example: infected prostate, lung, breast, lymph, gastrointestinal (e.g., colon) and genitourinary tract (e.g., kidney, epithelial cells), pharynx. Squamous cell carcinoma of lung includes malignant tumors, for example, most of colon cancer, rectal cancer, renal cell carcinoma, liver cancer, non-small cell cancer of lung, small intestine cancer and esophageal cancer. Metastatic lesions of the above cancers can likewise be treated and prevented using the methods and compositions of the invention.

### DETAILED DESCRIPTION OF THE INVENTION

It is well known that the α chain variable domain and the β chain variable domain of a TCR contain three CDRs (similar to the complementarity determining regions of antibodies), respectively. CDR3 interacts with the antigen short peptide, and CDR1 and CDR2 interact with HLA. Therefore, the CDR of a TCR molecule determines its interaction with the antigen short peptide-HLA complex. The amino acid sequences of α chain variable domain and β chain variable domain of a wild type TCR capable of binding the complex of antigen short peptide VLDGLDVLL and HLA-A0201 (i.e., VLDGLDVLL-HLA-A0201 complex) are SEQ ID NO: 1 and SEQ ID NO: 2, respectively. This sequence was firstly discovered by the inventors. It has the following CDR regions:

α chain variable domain CDR CDR1: DRGSQS
CDR2α: IYSNGD
CDR3α: AVARTYTGNQFY

and β chain variable domain CDR CDR1β: SEHNR
CDR2β: FQNEAQ
CDR3β: ASSSQKFSGIQPQH.

In the present invention, a high affinity TCR is obtained by subjecting mutation and screen in the above CDR regions, which has an affinity for VLDGLDVLL-HLA-A0201 complex that is at least 2 times greater than that of a wild type TCR for VLDGLDVLL-HLA-A0201 complex.

In the present invention, a T cell receptor (TCR) is provided, which has an activity in binding to VLDGLDVLL-HLA-A0201 complex.

The T cell receptor comprises a TCR α chain variable domain and a TCR β chain variable domain, the TCRα chain variable domain comprises three CDR regions, and the reference sequences of the three CDR regions of the TCR α chain variable domain are listed as follows,

CDR1α: DRGSQS

CDR2α: IYSNGD

CDR3α: AVARTYTGNQFY, and contains at least one of the following mutations:

| Residue before mutation | Residue after mutation |
|---|---|
| S at position 4 of CDR1α | T or A |
| S at position 6 of CDR1α | A |
| I at position 1 of CDR2α | Q or L or T |
| Y at position 2 of CDR2α | V |
| S at position 3 of CDR2α | M or V or Q |
| N at position 4 of CDR2α | P or D |
| A at position 3 of CDR3α | V |
| R at position 4 of CDR3α | L |
| T at position 5 of CDR3α | S |
| Y at position 6 of CDR3α | W |
| T at position 7 of CDR3α | K or A or R or L or Q or F |
| G at position 8 of CDR3α | S |
| N at position 9 of CDR3α | T |
| Q at position 10 of CDR3α | G or R |

and/or, the TCR β chain variable domain comprises three CDR regions, and the reference sequences of the three CDR regions of the TCR β chain variable domain are listed as follows,

CDR1β: SEHNR

CDR2β: FQNEAQ

CDR3β: ASSSQKFSGIQPQH, and contains at least one of the following mutations:

| Residue before mutation | Residue after mutation |
|---|---|
| N at position 3 of CDR2β | D or G |
| E at position 4 of CDR2β | S or R |
| A at position 5 of CDR2β | I or S |
| Q at position 6 of CDR2β | E |
| S at position 3 of CDR3β | N |
| S at position 4 of CDR3β | A or P or N or K or Q or T or M or R |
| Q at position 5 of CDR3β | S or G or T |
| K at position 6 of CDR3β | P or G or L |
| F at position 7 of CDR3β | V or L. |

In another preferred embodiment, the number of mutations in the CDR region of the TCR α chain may be 1, 2, 3, 4, 5, 6, 7, 8, 9, 10, 11 or 12.

In another preferred embodiment, the number of mutations in the CDR region of the TCR β chain may be 1, 2, 3, 4, 5, 6, 7 or 8.

Moreover, the TCR of the present invention is an αβ heterodimeric TCR, and the α chain variable domain of the TCR comprises an amino acid sequence having at least 85%, preferably at least 90%; more preferably, at least 92%; most preferably, at least 94% (e.g., may be at least 88%, 89%, 90%, 91%, 92%, 93%, 94%, 95%, 96%, 97%, 98%, 99% of sequence homology) of sequence homology with the amino acid sequence shown in SEQ ID NO: 1; and/or the β chain variable domain of the TCR comprises an amino acid sequence having at least 90%, preferably at least 92%; more preferably, at least 94%; most preferably at least 97% (e.g., may be at least 91%, 92%, 93%, 94%, 95%, 96%, 97%, 98%, 99% of sequence homology) of sequence homology with the amino acid sequence shown in SEQ ID NO: 2.

Additionally, the TCR of the present invention is a single-chain TCR, and the α chain variable domain of the TCR comprises an amino acid sequence having at least 85%, preferably at least 90%; more preferably, at least 92%; most preferably at least 94% (e.g., may be at least 88%, 89%, 90%, 91%, 92%, 93%, 94%, 95%, 96%, 97%, 98%, 99% of sequence homology) of sequence homology with the amino acid sequence shown in SEQ ID NO: 3; and/or the β chain variable domain of the TCR comprises an amino acid sequence having at least 90%, preferably at least 92%; more preferably, at least 94%; most preferably at least 97% (e.g., may be at least 91%, 92%, 93%, 94%, 95%, 96%, 97%, 98%, 99% of sequence homology) of sequence homology with the amino acid sequence shown in SEQ ID NO: 4.

Preferably, the TCR comprises (i) all or part of TCR α chain other than its transmembrane domain, and (ii) all or part of TCR β chain other than its transmembrane domain, wherein both of (i) and (ii) comprise the variable domain and at least a portion of constant domains of the TCR chain.

In the present invention, the three CDRs of α chain variable domain SEQ ID NO: 1 of the wild type TCR, i.e., CDR1, CDR2 and CDR3 are located at positions 27-32, 50-55 and 90-101 of SEQ ID NO: 1, respectively. Accordingly, the amino acid residue is numbered as shown in SEQ ID NO: 1, 27D is D at the 1^{st} position of CDR1α, 28R is R at the 2^{nd} position of CDR1α, 29G is G at the 3^{rd} position of CDR1α, 30S is S at the 4^{th} position of CDR1α, 31Q is Q at the 5^{th} position of CDR1α, 32S is S at the 6^{th} position of CDR1α; 50I is I at the 1^{st} position of CDR2α, 51Y is Y at the 2^{nd} position of CDR2α, 52S is S at the 3^{rd} position of CDR2α, 53N is N at 4^{th} position of CDR2α, 54G is G at the 5^{th} position of CDR2α, 55D is D at the 6^{th} position of CDR2α; 90A is A at the 1^{st} position of CDR3α, 91V is V at the 2^{nd} position of CDR3α, 92A is A of the 3^{rd} position of CDR3α, 93R is R at the 4^{th} position of CDR3α, 94T is T at the 5^{th} position of CDR3α, 95Y is Y at the 6^{th} position of CDR3α, 96T is T at the 7^{th} position of CDR3α, 97G is G at the 8^{th} position of CDR3α, 98N is N at the 9^{th} position of CDR3α, 99Q is Q at the 10^{th} position of CDR3α, 100F is F at the 11^{th} position of CDR3α, and 101Y is Y at the 12^{th} position of CDR3α.

Similarly, in the present invention, the three CDRs of β chain variable domain SEQ ID NO: 2 of the wild type TCR, i.e., CDR1, CDR2 and CDR3 are located at positions 27-31, 49-54 and 93-106 of SEQ ID NO: 2, respectively. Accordingly, the amino acid residue is numbered as shown in SEQ ID NO: 2, 51N is N at the 3^{rd} position of CDR2β, 52E is E at the 4^{th} position of CDR2β, 53A is A at the 5^{th} position of CDR2β, 54Q is Q at the 6^{th} position of CDR2β, 95S is S at the 3^{rd} position of CDR3β, 96S is S at the 4^{th} position of CDR3β, and 98K is K at the 6^{th} position of CDR3β.

The present invention provides a TCR having the property of binding to VLDGLDVLL-HLA-A0201 complex, and comprises an α chain variable domain and a β chain variable domain, wherein the TCR has a mutation in the α chain variable domain shown in SEQ ID NO: 1, and the site of the mutated amino acid residue includes one or more of 30S, 32S, 50I, 51Y, 52S, 53N, 92A, 93R, 94T, 95Y, 96T, 97G, 98N and 99Q, wherein the amino acid residue is numbered as shown in SEQ ID NO: 1; and/or the TCR has a mutation in the β chain variable domain shown in SEQ ID NO: 2, and the site of the mutated amino acid residue includes one or more of 51N, 52E, 53A, 54Q, 95S, 96S, 97Q, 98K and 99F, wherein the amino acid residue is numbered as shown in SEQ ID NO: 2;

Preferably, the mutated TCR α chain variable domain comprises one or more amino acid residues selected from the group consisting of: 30T or 30A; 32A; 50Q, SOL or 50T; 51V; 52M, 52V, 52Q; 53P or 53D; 92V; 93L; 94S; 95W; 96K, 96A, 96R, 96L, 96Q, 96F or 97S; 98T; and 99R or 99G; wherein the amino acid residue is numbered as shown in SEQ ID NO: 1; and/or the mutated TCR β chain variable domain comprises one or more amino acid residues selected from the group consisting of: 51D or 51G; 52S or 52R; 531 or 53S; 54E; 95N; 96A, 96P, 96N, 96K, 96Q, 96T, 96M or 96R; 97S, 97G or 97T; 98G, 98P or 98L; 99V or 99L; wherein the amino acid residue is numbered as shown in SEQ ID NO: 2.

More specifically, in the α chain variable domain, specific forms of the mutation include one or more groups of S30T/A, S32A, I50Q/L/T, Y51V, S52M/V/Q, N53P/D, A92V, R93L, T94S, Y95W, T96K/A/R/L/Q/F, G97S, N98T, Q99R/G; and in the β chain variable domain, specific forms of the mutation include one or more groups of N51D/G, E52S/R, A53I/S, Q54E, S95N, S96A/P/N/K/Q/T/M/R, Q97S/G/T, K98G/P/L, F99V/L.

Thr48 of the wild type TCR α chain constant region TRAC*01 exon 1 was mutated to cysteine according to the site-directed mutagenesis method well known to a skilled person in the art, and Ser57 of the β chain constant region TRBC1*01 or TRBC2*01 exon 1 was mutated to cysteine, so as to obtain a reference TCR, the amino acid sequences of which are shown in FIGS. 8a and 8b, respectively, and the mutated cysteine residues are indicated by bold letters. The above cysteine substitutions can form an artificial interchain disulfide bond between the constant regions of α and β chain of the reference TCR to form a more stable soluble TCR, so that it is easier to evaluate the binding affinity and/or binding half-life between TCR and VLDGLDVLL-HLA-A0201 complex. It will be appreciated that the CDR regions of the TCR variable region determine its affinity for pMHC complex, therefore, the above cysteine substitutions in the TCR constant region won't affect the binding affinity and/or binding half-life of TCR. Therefore, in the present invention, the measured binding affinity between the reference TCR and VLDGLDVLL-HLA-A0201 complex is considered to be the binding affinity between the wild-type TCR and VLDGLDVLL-HLA-A0201 complex. Similarly, if the binding affinity between the TCR of the invention and VLDGLDVLL-HLA-A0201 complex is determined to be at least 10 times the binding affinity between the reference TCR and VLDGLDVLL-HLA-A0201 complex, the binding affinity between the TCR of the present invention and VLDGLDVLL-HLA-A0201 complex is at least 10 times the binding affinity between the wild type TCR and VLDGLDVLL-HLA-A0201 complex.

The binding affinity (in inverse proportion to the dissociation equilibrium constant K_{D}) and the binding half-life (expressed as T_{1/2}) can be determined by any suitable method. It should be understood that doubling of the affinity of the TCR will halve K_{D}. T_{1/2} is calculated as In2 divided by dissociation rate (K_{of}). Therefore, doubling of T_{1/2} will halve K_{off}. Preferably, the binding affinity or binding half-life of a given TCR is detected for several times by using the same test protocol, for example 3 or more times, and the average of the results is taken. In a preferred embodiment, these measurements are performed using the surface plasmon resonance (BIAcore) method in the Examples herein. The dissociation equilibrium constant K_{D} of the reference TCR to VLDGLDVLL-HLA-A0201 complex is detected as 1.10E-05M, that is, 11 μM by the method, and in the present invention, the dissociation equilibrium constant K_{D} of the wild type TCR to VLDGLDVLL-HLA-A0201 complex is also considered as 11 μM. Since doubling of the affinity of TCR will halve K_{D}, if the dissociation equilibrium constant K_{D} of the high affinity TCR to VLDGLDVLL-HLA-A0201 complex is detected as 1.10E-06M, i.e., 1.1 μM, the affinity of the high affinity TCR for VLDGLDVLL-HLA-A0201 complex is 10 times that of the wild type TCR for VLDGLDVLL-HLA-A0201 complex. A skilled person is familiar with the conversion relationship between K_{D} value units, i.e., 1 M=1000 μM, 1 μM=1000 nM, and 1 nM=1000 pM.

In a preferred embodiment of the invention, the affinity of the TCR for VLDGLDVLL-HLA-A0201 complex is at least 2 times that of the wild type TCR; preferably at least 5 times; more preferably at least 10 times.

In another preferred embodiment, the affinity of the TCR for VLDGLDVLL-HLA-A0201 complex is at least 50 times that of the wild type TCR; preferably, at least 100 times; more preferably, at least 500 times; most preferably, at least 1000 times.

In another preferred embodiment, the affinity of the TCR for VLDGLDVLL-HLA-A0201 complex is at least 10⁴ times; preferably, at least 10⁵ times that of the wild type TCR.

Specifically, the dissociation equilibrium constant K_{D} of the TCR for VLDGLDVLL-HLA-A0201 complex is ≤5 μM;

In another preferred embodiment, the dissociation equilibrium constant of the TCR for VLDGLDVLL-HLA-A0201 complex is 10 nM<K_{D}<50 nM; preferably, 50 nM≤K_{D}≤500 nM; more preferably, 100 nM≤K_{D}≤500 nM;

In another preferred embodiment, the dissociation equilibrium constant of the TCR for VLDGLDVLL-HLA-A0201 complex is 50 pM<K_{D}≤500 pM; preferably, 50 pM≤K_{D}≤100 pM.

Mutations can be carried out by any suitable method including, but not limited to, those based on polymerase chain reaction (PCR), restriction enzyme-based cloning or linkage-independent cloning (LIC) methods. Many standard molecular biology textbooks describe these methods in detail. More details about polymerase chain reaction (PCR) mutagenesis and cloning based on restriction enzymes can be found in Sambrook and Russell, (2001) Molecular Cloning-A Laboratory Manual (Third Edition) CSHL Publishing house. More information about LIC method can be found in Rashtchian, (1995) Curr Opin Biotechnol 6(1): 30-6.

The method for producing the TCR of the present invention may be, but not limited to, screening for a TCR having high affinity for VLDGLDVLL-HLA-A2 complex from a diverse library of phage particles displaying such TCRs, as described in a literature (Li, et al). (2005) Nature Biotech 23(3): 349-354).

It will be appreciated that genes expressing amino acid of α and β chain variable domain of a wild-type TCR or genes expressing amino acid of α and β chain variable domain of a slightly modified wild-type TCR can be used to prepare template TCRs. Changes necessary to produce the high affinity TCR of the invention are then introduced into the DNA encoding the variable domain of the template TCR.

The high affinity TCR of the present invention comprises one of α chain variable domain amino acid sequences of SEQ ID NO: 57, 58, 59, 60, 61, 62, 63, 64, 65, 66, 67, 68, 69, 70, 71, 72, 73, 74, 75, 76, 77, 78, 79, 80, 81, 82 and/or one of (3 chain variable domain amino acid sequences of SEQ ID NO: 83, 84, 85, 86, 87, 88, 89, 90, 91, 92, 93, 94, 95, 96, 97, 98, 99, 100. Therefore, a TCR α chain comprising α chain variable domain amino acid sequence of the wild-type TCR (SEQ ID NO: 1) can bind to a TCR β chain comprising one of SEQ ID NO: 83, 84, 85, 86, 87, 88, 89, 90, 91, 92, 93, 94, 95, 96, 97, 98, 99, 100 to form a heterodimeric TCR or a single-chain TCR molecule. Alternatively, a TCR β chain comprising β variable domain amino acid sequence of the wild type TCR (SEQ ID NO: 2) can bind to a TCR a chain comprising one of SEQ ID NO: 57, 58, 59, 60, 61, 62, 63, 64, 65, 66, 67, 68, 69, 70, 71, 72, 73, 74, 75, 76, 77, 78, 79, 80, 81, 82 to form a heterodimeric TCR or single-chain TCR molecule. Alternatively, a TCR α chain comprising one of TCR α chain variable domain amino acid sequences SEQ ID NO: 57, 58, 59, 60, 61, 62, 63, 64, 65, 66, 67, 68, 69, 70, 71, 72, 73, 74, 75, 76, 77, 78, 79, 80, 81, 82 can bind to a TCR β chain comprising one of TCR β chain variable domain amino acid sequences SEQ ID NO: 83, 84, 85, 86, 87, 88, 89, 90, 91, 92, 93, 94, 95, 96, 97, 98, 99, 100 to form a heterodimeric TCR or a single-chain TCR molecule. In the present invention, the amino acid sequences of the α chain variable domain and β chain variable domain which form the heterodimeric TCR molecule are preferably selected from the following Table 1:

TABLE 1

| | α chain variable domain | β chain variable domain |
|---|---|---|
| TCR No. | sequence SEQ ID NO: | sequence SEQ ID NO: |
| 1 | 57 | 2 |
| 2 | 58 | 2 |
| 3 | 59 | 2 |
| 4 | 60 | 2 |
| 5 | 61 | 2 |
| 6 | 62 | 2 |
| 7 | 63 | 2 |
| 8 | 1 | 83 |
| 9 | 1 | 84 |
| 10 | 1 | 85 |
| 11 | 1 | 86 |
| 12 | 1 | 87 |
| 13 | 1 | 88 |
| 14 | 1 | 89 |
| 15 | 1 | 90 |
| 16 | 1 | 91 |
| 17 | 1 | 92 |
| 18 | 64 | 93 |
| 19 | 65 | 94 |
| 20 | 66 | 95 |
| 21 | 67 | 96 |
| 22 | 68 | 97 |
| 23 | 69 | 93 |
| 24 | 70 | 95 |
| 25 | 71 | 98 |
| 26 | 72 | 93 |
| 27 | 73 | 93 |
| 28 | 74 | 99 |
| 29 | 75 | 98 |
| 30 | 76 | 98 |
| 31 | 77 | 100 |
| 32 | 78 | 97 |
| 33 | 79 | 98 |
| 34 | 80 | 93 |
| 35 | 81 | 95 |
| 36 | 82 | 97 |

For the purposes of the present invention, the TCR of the invention is a moiety having at least one TCR α and/or TCR β chain variable domain. They usually comprise both of TCR α chain variable domain and TCR β chain variable domain. They may be αβ heterodimers or single-chain forms or any other stable forms. In adoptive immunotherapy, the full length chain of the αβ heterodimeric TCR (including the cytoplasmic and transmembrane domains) can be transfected. The TCR of the present invention can be used as a targeting agent for delivering a therapeutic agent to an antigen presenting cell or in combination with other molecules to prepare a bifunctional polypeptide to direct effector cells, when the TCR is preferably in a soluble form.

For stability, it is disclosed in the prior art that a soluble and stable TCR molecule can be obtained by introducing an artificial interchain disulfide bond between the α and β chain constant domains of a TCR, as described in PCT/CN2015/093806. Therefore, the TCR of the invention may be a TCR that an artificial interchain disulfide bond is introduced between the residues of its α and β chain constant domains. Cysteine residues form an artificial interchain disulfide bond between the α and β chain constant domains of the TCR. A cysteine residue can replace other amino acid residue at a suitable position in a native TCR to form an artificial interchain disulfide bond. For example, Thr48 of TRAC*01 exon 1 and Ser57 of TRBC1*01 or TRBC2*01 exon 1 can be replaced to form a disulfide bond. Other sites for introducing a cysteine residue to form a disulfide bond may be: Thr45 of TRAC*01 exon 1 and Ser77 of TRBC1*01 or TRBC2*01 exon 1; Tyr10 of of TRAC*01 exon 1 and Ser17 of TRBC1*01 or TRBC2*01 exon 1; Thr45 of TRAC*01 exon 1 and Asp59 of TRBC1*01 or TRBC2*01 exon 1; Ser15 of TRAC*01 exon 1 and Glu15 of TRBC1*01 or TRBC2*01 exon 1; Arg53 of TRAC*01 exon 1 and Ser54 of TRBC1*01 or TRBC2*01 exon 1; Pro89 of TRAC*01 exon 1 and Ala19 of TRBC1*01 or TRBC2*01 exon 1; or Tyr10 of TRAC*01 exon 1 and Glu20 of TRBC1*01 or TRBC2*01 exon 1. That is, cysteine residues replace any group of the above-mentioned sites in α and β chain constant domains. A maximum of 15, or a maximum of 10, or a maximum of 8 or fewer amino acids may be truncated at one or more C-termini of the constant domain of the TCR of the invention such that it does not include cysteine residues to achieve the purpose of deleting native interchain disulfide bonds, or the cysteine residues forming a natural interchain disulfide bond can also be mutated to another amino acid for achieving the above purpose.

As described above, the TCR of the present invention may comprise an artificial interchain disulfide bond introduced between residues of its α and β chain constant domains. It should be noted that the introduced artificial disulfide bond as described above can be contained or not contained between the constant domains, and the TCR of the present invention may contain a TRAC constant domain sequence and a TRBC1 or TRBC2 constant domain sequence. The TRAC constant domain sequence and the TRBC1 or TRBC2 constant domain sequence of the TCR can be joined by a natural interchain disulfide bond present in the TCR.

Additionally, as for stability, it was also disclosed in a patent literature PCT/CN2016/077680 that the introduction of an artificial interchain disulfide bond between α chain variable region and β chain constant region of a TCR can significantly improve the stability of the TCR. Therefore, an artificial interchain disulfide bond may be contained between α chain variable region and β chain constant region of a high affinity TCR of the present invention. Specifically, cysteine residues forming an artificial interchain disulfide bond between α chain variable region and β chain constant region of the TCR is substituted for: an amino acid at position 46 of TRAV and amino acid at position 60 of TRBC1*01 or TRBC2*01 exon 1; an amino acid at position 47 of TRAV and amino acid at position 61 of TRBC1*01 or TRBC2*01 exon 1; amino acid at position 46 of TRAV and amino acid at position 61 of TRBC1*01 or TRBC2*01 exon 1; or an amino acid at position 47 of TRAV and amino acid at position 60 of TRBC1*01 or TRBC2*01 exon 1. Preferably, such a TCR may comprises (i) all or part of TCR α chain other than its transmembrane domain, and (ii) all or part of TCR β chain other than its transmembrane domain, wherein both of (i) and (ii) comprise the variable domain and at least a portion of constant domains of the TCR chain, and the α chain and β chain form a heterodimer. More preferably, such TCR may comprise α chain variable domain and β chain variable domain and all or part of β chain constant domain other than the transmembrane domain, which, however, does not comprise α chain constant domain, and the α chain variable domain of the TCR and the β chain form a heterodimer.

For stability, in another aspect, the TCR of the present invention also includes a TCR having a mutation in its hydrophobic core region, and these mutations in hydrophobic core region are preferably mutations capable of increasing the stability of the TCR of the present invention, as described in WO 2014/206304. Such a TCR can have mutations at following positions in the variable domain hydrophobic core: (α and/or β chain) variable region amino acids at position 11, 13, 19, 21, 53, 76, 89, 91, 94, and/or α chain J gene (TRAJ) short peptide amino acid at reciprocal positions 3, 5, 7 and/or β chain J gene (TRBJ) short peptide amino acid at reciprocal positions 2, 4, 6, wherein the positions in amino acid sequence are numbered according to the position numbers listed in the International Immunogenetics Information System (IMGT). A skilled person in the art will know the above-described international immunogenetic information system and can obtain the position numbers of the amino acid residues of different TCRs in the IMGT based on the database.

More specifically, in the present invention, a TCR in which there is a mutation in the hydrophobic core region may be a high-stability single-chain TCR consisting of TCR α and β chain variable domains that linked by a flexible peptide chain. The CDR regions of TCR variable region determine its affinity for the short peptide-HLA complex, and mutations in hydrophobic core can increase the stability of the TCR, but won't affect its affinity for the short peptide-HLA complex. It should be noted that the flexible peptide chain in the present invention may be any peptide chain suitable for linking TCR α and β chain variable domains. The template chain constructed in Example 1 of the present invention for screening high-affinity TCRs is a high-stability single-chain TCR containing mutations in hydrophobic core as described above. The affinity between a TCR and VLDGLDVLL-HLA-A2 complex can be easily evaluated by using a TCR with higher stability.

The CDR regions of α chain variable domain and β chain variable domain of the single chain template TCR are identical to the CDR regions of the wild type TCR. That is, the three CDRs of α chain variable domain are CDR1α: DRGSQS, CDR2α: IYSNGD, and CDR3α: AVARTYTGNQFY and and the three CDRs of β chain variable domains are CDR1β: SEHNR, CDR2β: FQNEAQ, and CDR3β: ASSSQKFSGIQPQH, respectively. The amino acid sequence (SEQ ID NO: 53) and nucleotide sequence (SEQ ID NO: 54) of the single-chain template TCR are shown in FIGS. 7a and 7b, respectively, thereby screening a single-chain TCR consisting of α-chain variable domain and β-chain variable domain and having high affinity for VLDGLDVLL-HLA-A0201 complex.

In the present invention, the three CDRs of α chain variable domain of the single-chain template TCR (SEQ ID NO: 3), i.e., CDR1, CDR2 and CDR3 are located at positions 27-32, 50-55 and 90-101 of SEQ ID NO: 3, respectively. Accordingly, the amino acid residues are numbered according to the number as shown in SEQ ID NO: 1. 27D is D at the 1^{st} position of CDR1α, 28R is Rat the 2^{nd} position of CDR1α, 29G is G at the 3^{rd} position of CDR1α, 30S is S at the 4^{th} position of CDR1α, 31Q is Q at the 5^{th} position of CDR1α, 32S is S at the 6^{th} position of CDR1α, 50I is I at the 1^{st} position of CDR2α, 51YI is Y at the 2^{nd} position of CDR2α, 52S is S at the 3^{rd} position of CDR2α, 53N is N at the 4^{th} position of CDR2α, 54G is G at the 5^{th} position of CDR2α, 55D is D at the 6^{th} position of CDR2α, 90A is A at the 1^{st} position of CDR3α, 91V is V at the 2^{nd} position of CDR3α, 92A is A at the 3^{nd} position of CDR3α, 93R is R at the 4^{th} position of CDR3α, 94T is T at the 5^{th} position of CDR3α, 95Y is Y at the 6^{th} position of CDR3α, 96I is I at the 7^{th} position of CDR3α, 97G is G at the 8^{th} position of CDR3α, 98N is N at the 9^{th} position of CDR3α, 99Q is Q at the 10^{th} position of CDR3α, 100F is F at the 11^{th} position of CDR3α and 101Y is Y at the 12^{th} position of CDR3α.

Similarly, in the present invention, the three CDRs of β chain variable domain of the single-chain template TCR (SEQ ID NO: 4), i.e., CDR1, CDR2 and CDR3 are located at positions 27-31, 49-54 and 93-106 of SEQ ID NO: 2, respectively. Therefore, the amino acid residues are numbered according to the number as shown in SEQ ID NO: 4. 51N is N at the 3^{rd} position of CDR2β, 52E is E at the 4^{th} position of CDR2β, 53A is A at the 5^{th} position of CDR2β, 54Q is Q at the 6^{th} position of CDR2β, 95S is S at the 3^{rd} position of CDR3β, 96S is S at the 4^{th} position of CDR3β, and 98K is K at the 6^{th} position of CDR3β.

The αβ heterodimer of the present invention having high affinity for VLDGLDVLL-HLA-A2 complex is obtained by transferring the CDR regions of α and β chain variable domains of the selected high affinity single-chain TCR to the corresponding positions of α chain variable domain (SEQ ID NO: 1) and 13 chain variable domain (SEQ ID NO: 2) of a wild type TCR.

The high affinity TCR of the invention further comprises one of α chain variable domain amino acid sequences of SEQ ID NO: 9, 10, 11, 12, 13, 14, 15, 16, 17, 18, 19, 20, 21, 22, 23, 24, 25, 26, 27, 28, 29, 30, 31, 32, 33 and 34, and/or one of β chain variable domain amino acid sequences of SEQ ID NO: 35, 36, 37, 38, 39, 40, 41, 42, 43, 44, 45, 46, 47, 48, 49, 50, 51 and 52. Therefore, the α chain variable domain (SEQ ID NO: 3) of the above described high-stability single-chain TCR as a template chain can be combined with TCR β chain variable domain, the amino acid sequence of which is SEQ ID NO: 35, 36, 37, 38, 39, 40, 41, 42, 43, 44, 45, 46, 47, 48, 49, 50, 51 or 52, to form the single-chain TCR molecule. Alternatively, the β chain variable domain (SEQ ID NO: 4) of the above described high-stability single-chain TCR as a template chain can be combined with TCR α chain variable domain, the amino acid sequence of which is SEQ ID NO: 9, 10, 11, 12, 13, 14, 15, 16, 17, 18, 19, 20, 21, 22, 23, 24, 25, 26, 27, 28, 29, 30, 31, 32, 33 or 34, to form the single-chain TCR molecule. Alternatively, one of the TCR α chain variable domains SEQ ID NO: 9, 10, 11, 12, 13, 14, 15, 16, 17, 18, 19, 20, 21, 22, 23, 24, 25, 26, 27, 28, 29, 30, 31, 32, 33 and 34 can be combined with one of the TCR β chain variable domains SEQ ID NO: 35, 36, 37, 38, 39, 40, 41, 42, 43, 44, 45, 46, 47, 48, 49, 50, 51 and 52 to form the single-chain TCR molecule. In the present invention, the amino acid sequences of α chain variable domain and β chain variable domain of the high-affinity single-chain TCR molecule are preferably selected from the following Table 2:

TABLE 2

| | α chain variable domain | β chain variable domain |
|---|---|---|
| TCR No. | sequence SEQ ID NO: | sequence SEQ ID NO: |
| s-1 | 9 | 4 |
| s-2 | 10 | 4 |
| s-3 | 11 | 4 |
| s-4 | 12 | 4 |
| s-5 | 13 | 4 |
| s-6 | 14 | 4 |
| s-7 | 15 | 4 |
| s-8 | 3 | 35 |
| s-9 | 3 | 36 |
| s-10 | 3 | 37 |
| s-11 | 3 | 38 |
| s-12 | 3 | 39 |
| s-13 | 3 | 40 |
| s-14 | 3 | 41 |
| s-15 | 3 | 42 |
| s-16 | 3 | 43 |
| s-17 | 3 | 44 |
| s-18 | 16 | 45 |
| s-19 | 17 | 46 |
| s-20 | 18 | 47 |
| s-21 | 19 | 48 |
| s-22 | 20 | 49 |
| s-23 | 21 | 45 |
| s-24 | 22 | 47 |
| s-25 | 23 | 50 |
| s-26 | 24 | 45 |
| s-27 | 25 | 45 |
| s-28 | 26 | 51 |
| s-29 | 27 | 50 |
| s-30 | 28 | 50 |
| s-31 | 29 | 52 |
| s-32 | 30 | 49 |
| s-33 | 31 | 50 |
| s-34 | 32 | 45 |
| s-35 | 33 | 47 |
| s-36 | 34 | 49 |

The TCR of the present invention can be provided in a form of multivalent complex. The multivalent TCR complex of the present invention comprises a polymer formed by combining two, three, four or more TCRs of the present invention, for example, a tetrameric domain of p53 can be used to produce a tetramer. Alternatively, more TCRs of the invention can be combined with another molecule to form a complex. The TCR complexes of the invention can be used to track or target cells that present a particular antigen in vitro or in vivo, or produce intermediates of other multivalent TCR complexes with such uses.

The TCR of the present invention may be used alone or combined with a conjugate in a covalent manner or other manner, preferably in a covalent manner. The conjugate includes a detectable label (for diagnostic purposes, wherein the TCR is used to detect the presence of a cell presenting VLDGLDVLL-HLA-A2 complex), a therapeutic agent, a PK (protein kinase) modifying moiety, or combination of any of the above described substances.

Detectable labels for diagnostic purposes include, but are not limited to, fluorescent or luminescent labels, radioactive labels, MRI (magnetic resonance imaging) or CT (electron computed tomography) contrast agents, or enzymes capable of producing detectable products.

Therapeutic agents that can be combined with or coupled to the TCRs of the invention include, but are not limited to: 1. Radionuclides (Koppe et al., 2005, Cancer metastasis reviews 24, 539); 2. Biotoxin (Chaudhary et al., 1989, Nature 339, 394; Epel et al., 2002, Cancer Immunology and Immunotherapy 51, 565); 3. Cytokines, such as IL-2, etc. (Gillies et al., 1992, National Academy of Sciences (PNAS) 89, 1428; Card et al., 2004, Cancer Immunology and Immunotherapy 53, 345; Halin et al., 2003, Cancer Research 63, 3202); 4. Antibody Fc fragment (Mosquera et al., 2005, The Journal Of Immunology 174, 4381); 5. Antibody scFv fragments (Zhu et al., 1995, International Journal of Cancer 62, 319); 6. Gold nanoparticles/Nanorods (Lapotko et al., 2005, Cancer letters 239, 36; Huang et al., 2006, Journal of the American Chemical Society 128, 2115); 7. Viral particles (Peng et al., 2004, Gene therapy 11, 1234); 8. Liposomes (Mamot et al., 2005, Cancer research 65, 11631); 9. Nanomagnetic particles; 10. Prodrug activating enzymes (e.g., DT-diaphorase (DTD) or biphenyl hydrolase-like protein (BPHL); 11. chemotherapeutic agent (e.g., cisplatin) or any form of nanoparticles, and the like.

An antibody to which the TCR of the present invention binds or a fragment thereof includes an anti-T cell or an NK-cell determining antibody, such as an anti-CD3 or anti-CD28 or anti-CD16 antibody, and the above antibody or a fragment thereof binds to a TCR, thereby better directing effector cells to target cells. In a preferred embodiment, the TCR of the invention binds to an anti-CD3 antibody or a functional fragment or variant thereof. Specifically, a fusion molecule of the TCR of the present invention and an anti-CD3 single-chain antibody comprises a TCR α chain variable domain, the amino acid sequence of which is selected from the group consisting of SEQ ID NO: 9, 10, 11, 12, 13, 14, 15, 16, 17, 18, 19, 20, 21, 22, 23, 24, 25, 26, 27, 28, 29, 30, 31, 32, 33, 34, 35, 36, 37, 58, 59, 60, 61, 62, 63, 64, 65, 66, 67, 68, 69, 70, 71, 72, 73, 74, 75, 76, 77, 78, 79, 80, 81 and 82, and a TCR β chain variable domain, the amino acid sequence of which is selected from the group consisting of SEQ ID NO: 35, 36, 37, 38, 39, 40, 41, 42, 43, 44, 45, 46, 47, 48, 49, 50, 51, 52, 53, 87, 88 89, 90, 91, 92, 93, 94, 95, 96, 97, 98, 99 and 100.

The invention also relates to a nucleic acid molecule encoding the TCR of the invention. The nucleic acid molecule of the invention may be in a form of DNA or RNA. DNA can be a coding strand or a non-coding strand. For example, a nucleic acid sequence encoding the TCR of the invention may be the same as the nucleic acid sequence set forth in the Figures of the invention or a degenerate variant thereof. By way of example, “degenerate variant”, as used herein, refers to a nucleic acid sequence which encodes a protein with a sequence of SEQ ID NO: 53, but is differences from the sequence of SEQ ID NO: 54.

The full length sequence of the nucleic acid molecule of the present invention or a fragment thereof can generally be obtained by, but not limited to, PCR amplification, recombinant methods or synthetic methods. At present, it has been possible to obtain a DNA sequence encoding the TCR (or a fragment thereof, or a derivative thereof) of the present invention completely by chemical synthesis. And then the DNA sequence can be introduced into various existing DNA molecules (or vectors) and cells known in the art.

The invention also relates to vectors comprising the nucleic acid molecules of the invention, as well as host cells genetically engineered using the vectors or coding sequences of the invention.

The invention also encompasses isolated cells, particularly T cells, which express the TCR of the invention. There are a number of methods suitable for T cell transfection with DNA or RNA encoding the high affinity TCR of the invention (e.g., Robbins et al., (2008) J. Immunol. 180: 6116-6131). T cells expressing the high affinity TCR of the invention can be used in adoptive immunotherapy. A skilled person in the art can know many suitable methods for performing adoptive therapy (e.g., Rosenberg et al., (2008) Nat Rev Cancer 8(4): 299-308).

The invention also provides a pharmaceutical composition, comprising a pharmaceutically acceptable carrier and a TCR of the invention, or a TCR complex of the invention, or cells presenting the TCR of the invention.

The invention also provides a method for treating a disease, comprising administering to a subject in need thereof an appropriate amount of a TCR of the invention, or a TCR complex of the invention, or cells presenting a TCR of the invention, or a pharmaceutical composition of the invention.

It should be understood that the amino acid names herein are identified by internationally accepted single English letters, and the corresponding three-letter abbreviated names of an amino acid are: Ala (A), Arg (R), Asn (N), Asp (D), Cys (C), Gln (Q), Glu (E), Gly (G), His (H), Ile (I), Leu (L), Lys (K), Met (M), Phe (F), Pro (P), Ser (S), Thr (T), Trp (W), Tyr (Y), Val (V).

In the present invention, both of Pro60 or 60P represent proline at position 60. Further, regarding the expression of the specific form of mutation in the present invention, such as “T27G” represents that T at the 27^{th} position is substituted by G. Similarly, “I29A/V” means that I at the 29^{th} position is substituted by A or substituted by V, and so on.

In the art, when an amino acid with similar properties is used for substitution, the function of the protein is usually not altered. The addition of one or several amino acids at C-terminus and/or N-terminus generally does not alter the structure and function of the protein. Therefore, the TCR of the invention further includes a TCR, wherein up to 5, preferably up to 3, more preferably up to 2, the most preferably 1 amino acid (especially an amino acid located outside CDR regions) of the TCR of the invention is replaced by an amino acid with similar properties and still be able to maintain its function.

The present invention also includes a TCR obtained from the TCR of the present invention by slight modification. Form of modification (usually without altering the primary structure) includes: chemically derived forms of the TCR of the invention, such as acetylation or carboxylation. Modifications also include glycosylation, such as those TCRs produced by glycosylation modifications in the synthesis and processing or in further processing steps of the TCR of the invention. Such modification can be accomplished by exposing the TCR to an enzyme performing glycosylation (such as a mammalian glycosylation enzyme or a deglycosylation enzyme). Modification forms also include sequences having phosphorylated amino acid residues (such as phosphotyrosine, phosphoserine, phosphothreonine). Also included are TCRs that have been modified to enhance their antiproteolytic properties or optimize solubility properties.

The TCR, TCR complexes of the invention or T cells transfected by the TCRs of the invention can be provided in a pharmaceutical composition together with a pharmaceutically acceptable carrier. The TCR, multivalent TCR complex or cell of the invention is typically provided as part of a sterile pharmaceutical composition, which typically comprises a pharmaceutically acceptable carrier. The pharmaceutical composition can be of any suitable form (depending on the desired method for administration to a patient). It can be provided in a unit dosage form, usually in a sealed container, and can be provided as part of a kit. Such kit (but not required) includes instructions. It can include a plurality of said unit dosage form.

Furthermore, the TCR of the invention may be used alone or in combination with other therapeutic agents (e.g., formulated in the same pharmaceutical composition).

The pharmaceutical composition may also contain a pharmaceutically acceptable carrier. The term “pharmaceutically acceptable carrier” refers to a carrier for the administration of a therapeutic agent. The term refers to such pharmaceutical carriers which themselves do not induce the production of antibodies harmful to the individual receiving the composition and which are not excessively toxic after administration. These carriers are well known to a skilled person in the art. A full discussion of pharmaceutically acceptable excipients can be found in Remington's Pharmaceutical Sciences (Mack Pub. Co., N.J. 1991). Such carriers include, but are not limited to, saline, buffer, dextrose, water, glycerol, ethanol, adjuvants, and combinations thereof.

The pharmaceutically acceptable carrier in the therapeutic composition may contain a liquid such as water, saline, glycerol and ethanol. In addition, auxiliary substances such as wetting or emulsifying agents, pH buffering substances and the like may also be present in these carriers.

In general, the therapeutic compositions can be formulated as injectables, such as liquid solutions or suspensions; and solid forms such as liquid carriers, which may be suitable for being formulated in solution or suspension prior to injection.

Once a composition of the invention is formulated, it can be administered by conventional routes including, but not limited to, intraocular, intramuscular, intravenous, subcutaneous, intradermal, or topical administration, preferably parenteral, including subcutaneous, intramuscular or intravenous administration. A subject to be prevented or treated may be an animal; especially a human.

When the pharmaceutical composition of the present invention is used for actual treatment, pharmaceutical compositions of various dosage forms may be employed depending on the uses, preferably, an injection, an oral preparation, or the like.

These pharmaceutical compositions can be formulated by mixing, diluting or dissolving according to conventional methods, occasionally, suitable pharmaceutical additives can be added such as excipients, disintegrating agents, binders, lubricants, diluents, buffers, isotonicity Isotonicities, preservatives, wetting agents, emulsifiers, dispersing agents, stabilizers and co-solvents, and the formulation process can be carried out in a customary manner depending on the dosage form.

The pharmaceutical composition of the present invention can also be administered in the form of a sustained release preparation. For example, the TCR of the present invention can be incorporated into a pill or microcapsule in which the sustained release polymer is used as a carrier, and then the pill or microcapsule is surgically implanted into the tissue to be treated. Examples of the sustained-release polymer include ethylene-vinyl acetate copolymer, polyhydrometaacrylate, polyacrylamide, polyvinylpyrrolidone, methylcellulose, lactic acid polymer, lactic acid-glycolic acid copolymer or the like, preferably biodegradable polymer, such as lactic acid polymer and lactic acid-glycolic acid copolymer.

When the pharmaceutical composition of the present invention is used for actual treatment, the amount of the TCR or TCR complex of the present invention or the cell presenting the TCR of the present invention as an active ingredient may be reasonably determined based on the body weight, age, sex, and degree of symptoms of each patient to be treated, and ultimately by a doctor.

Main Advantages of the Invention:

(1) The affinity and/or binding half-life of the TCR of the present invention for VLDGLDVLL-HLA-A2 complex is at least 2 times, preferably at least 10 times that of a wild type TCR.

(2) The affinity and/or binding half-life of the TCR of the present invention for VLDGLDVLL-HLA-A2 complex is at least 100 times, preferably at least 1000 times, and more preferably up to 10⁴-10⁵ times that of a wild type TCR.

(3) Effector cells transduced with the high-affinity TCR of the present invention exhibit a strong killing effect on target cells.

The invention is further illustrated by the following specific examples. It is to be understood that these examples are for illustrative purposes only and are not intended to limit the scope of the invention. The experimental methods in the following examples which do not specify the specific conditions are usually performed under conventional conditions, for example, conditions described in Sambrook and Russell et al., Molecular Cloning-A Laboratory Manual (Third Edition) (2001) CSHL Publishing company, or in accordance with the conditions recommended by the manufacturer. Percentages and parts are by weight unless otherwise stated.

Materials and Method

The experimental materials used in the examples of the present invention can commercially available, unless otherwise specified, among which E. coli DH5α was purchased from Tiangen, E. coli BL21 (DE3) was purchased from Tiangen, E. coli Tuner (DE3) was purchased from Novagen, and plasmid pET28a was purchased from Novagen.

### Example 1. Generation of Stable Single-Chaind TCR Template Chains with Mutations in Hydrophobic Core

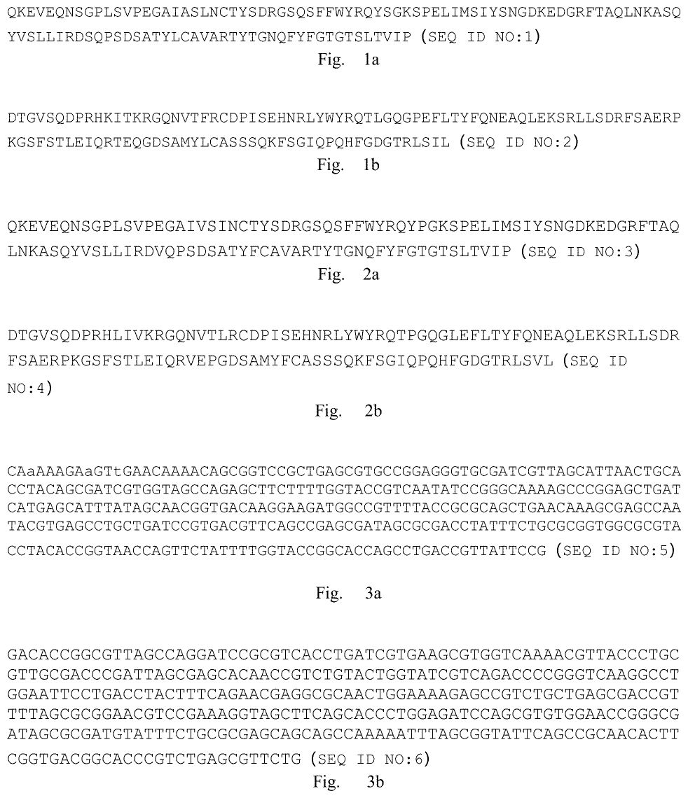

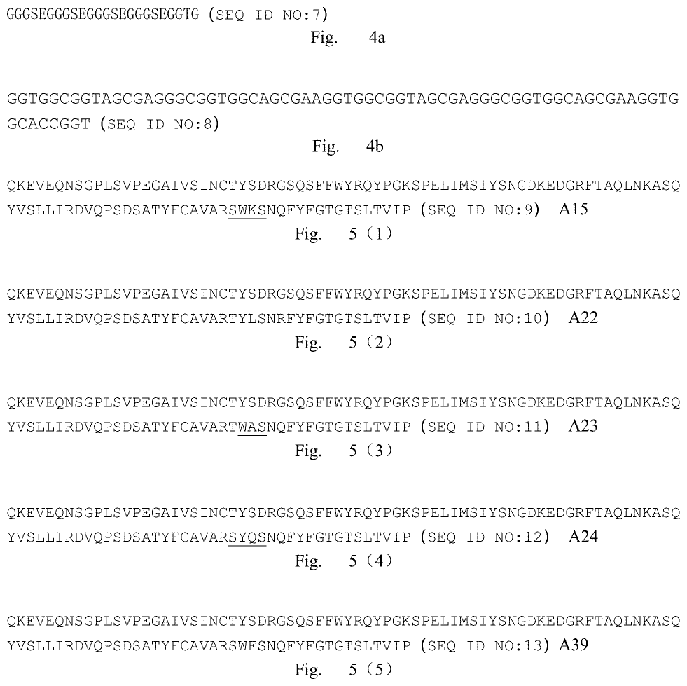

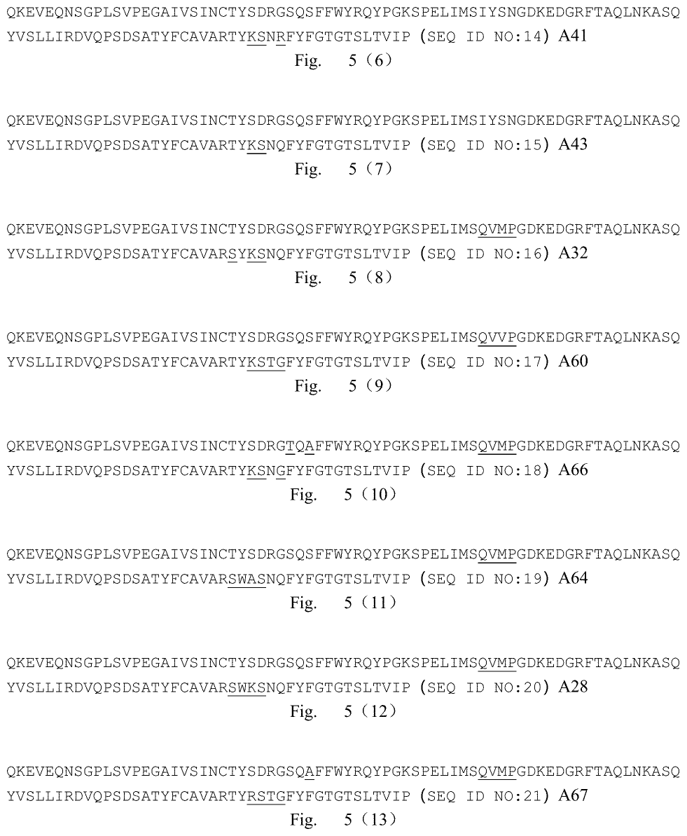

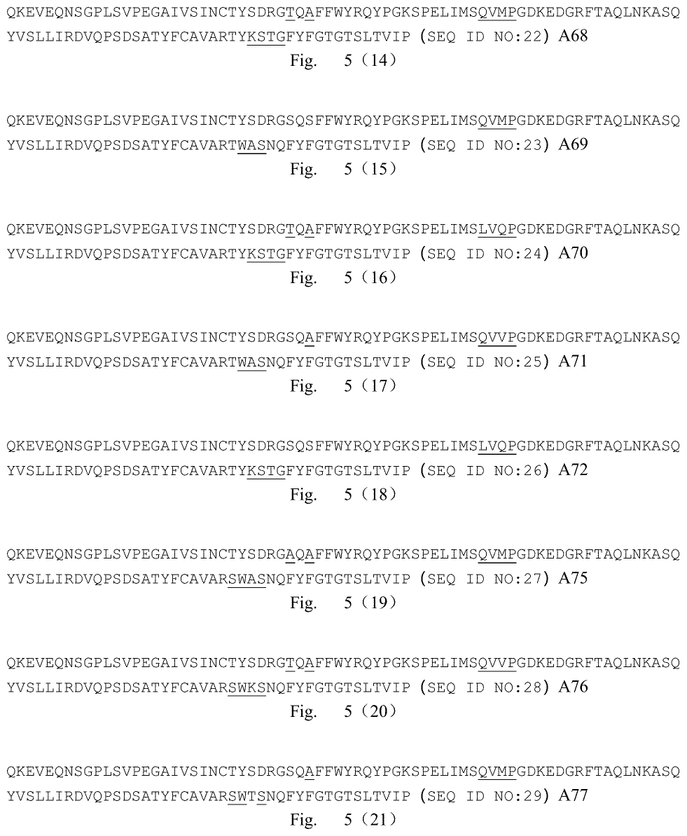

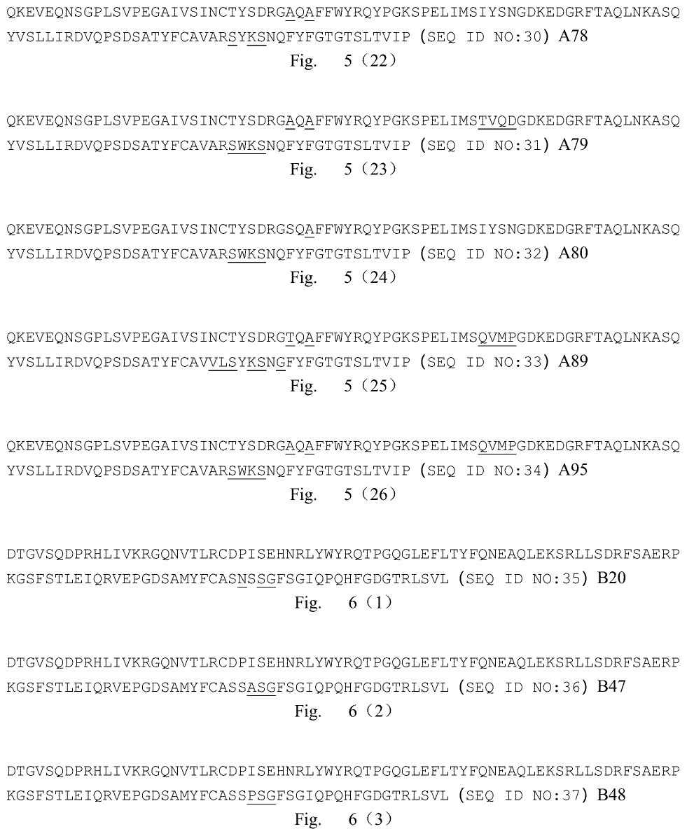

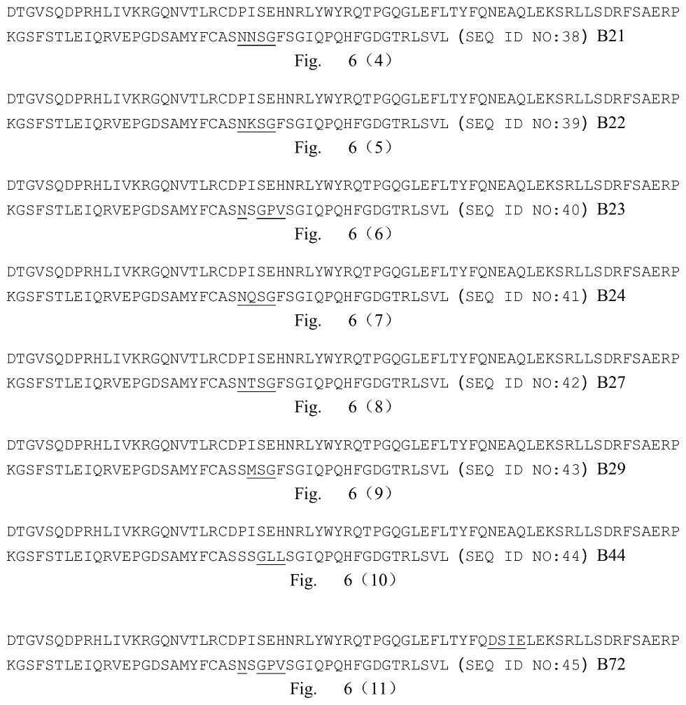

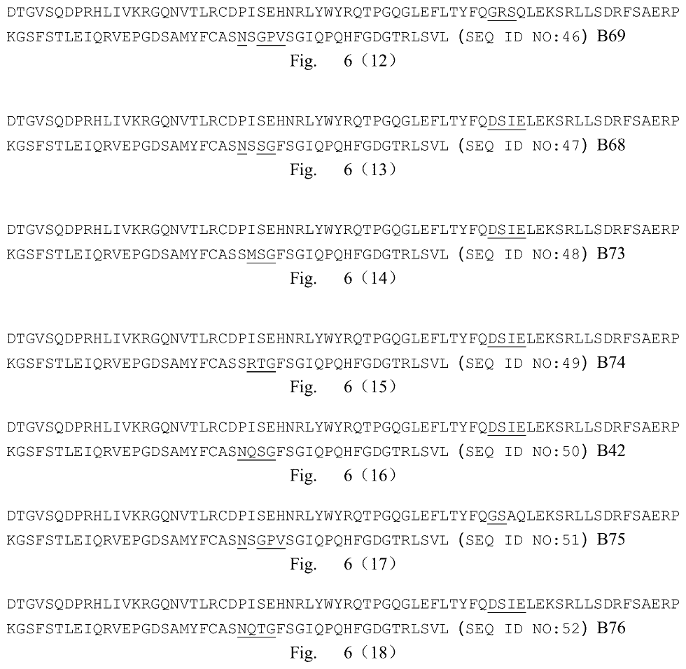

In the present invention, a method of site-directed mutagenesis was used according to a patent literture WO2014/206304 to construct a stable single-chain TCR molecule consisting of TCR α and β-chain variable domain connected by a flexible short peptide, and the amino acid and DNA sequences of which are SEQ ID NO: 53 and SEQ ID NO: 54, respectively, as shown in FIGS. 7a and 7b. The single-chain TCR molecule was used as a template for screening high-affinity TCR molecules. The amino acid sequences of a variable domain (SEQ ID NO: 3) and β variable domain (SEQ ID NO: 4) of the template chain are shown in FIGS. 2a and 2b; the corresponding DNA sequences are SEQ ID NO: 5 and 6, respectively, as shown in FIGS. 3a and 3b; and the amino acid sequence and DNA sequence of the flexible short linker are SEQ ID NOS: 7 and 8, respectively, as shown in FIGS. 4a and 4b.

The target gene carrying the template chain was digested with NcoI and NotI, and ligated with pET28a vector digested with NcoI and NotI. The ligation product was transformed into E. coli DH5α, plated on a kanamycin-containing LB plate, inverted and cultured at 37° C. overnight, and the positive clones were picked for PCR screening. Positive recombinants were sequenced to determine the correct sequence and the recombinant plasmid was extracted and transferred into E. coli BL21 (DE3) for expression.

### Example 2. Expression, Renaturation and Purification of the Stable Single-Chain TCR Constructed in Example 1

All of BL21 (DE 3) colonies containing the recombinant plasmid pET28a-template chain prepared in Example 1 were inoculated into LB medium containing kanamycin, and cultured at 37° C. until OD600 was 0.6-0.8. IPTG was added to a final concentration of 0.5 mM, and cultured at 37° C. for another 4 hrs. The cell pellets were harvested by centrifugation at 5000 rpm for 15 mins, and the cell pellets were lysed with Bugbuster Master Mix (Merck). The inclusion bodies were recovered by centrifugation at 6000 rpm for 15 min, followed by washing with Bugbuster (Merck) to remove cell debris and membrane fraction. The inclusion bodies were collected by centrifugation at 6000 rpm for 15 min, and dissolved in a buffer (20 mM Tris-HCl pH 8.0, 8 M urea), and the insoluble matters were removed by high-speed centrifugation. The supernatant was quantitativly determined by BCA method, and then dispensed and stored at −80° C. until use.

To 5 mg of dissolved single-chain TCR inclusion body protein, 2.5 mL of buffer (6 M Gua-HCl, 50 mM Tris-HCpH 8.1, 100 mM NaCl, 10 mM EDTA) was added, then DTT was added to a final concentration of 10 mM, and incubated at 37° C. for 30 min. The single-chain TCRs as treated above was added dropwise to a 125 mL of refolding buffer (100 mM Tris-HC1 pH 8.1, 0.4 M L-arginine, 5 M urea, 2 mM EDTA, 6.5 mM β-mercapthoethylamine, 1.87 mM Cystamine) with a syringe, and stirred at 4° C. for 10 min. Then the refolded solution was loaded into a cellulose membrane dialysis bag with a cut-off of 4 kDa, and the dialysis bag was placed in 1 L of pre-cooled water, and stirred slowly at 4° C. overnight. After 17 hours, the dialysis liquid was changed to 1 L of pre-chilled buffer (20 mM Tris-HCl pH 8.0) and dialysis was continued for 8 h at 4° C. The dialysis liquid was then replaced with the same fresh buffer and dialysis was continued overnight. After 17 hours, the sample was filtered through a 0.45 μm filter, vacuum degassed and purified through an anion exchange column (HiTrap Q HP, GE Healthcare) with a linear gradient elution of 0-1 M NaCl prepared with 20 mM Tris-HCl pH 8.0. The collected fractions were subjected to SDS-PAGE analysis, and the fractions containing single-chain TCRs were concentrated and further purified by a gel filtration column (Superdex 75 10/300, GE Healthcare), and the target components were also subjected to SDS-PAGE analysis.

The eluted fractions for BIAcore analysis was further tested for purity using gel filtration. The conditions were as follows: chromatographic column Agilent Bio SEC-3 (300 A, φ7.8×300 mm), mobile phase 150 mM phosphate buffer, flow rate 0.5 mL/min, column temperature 25° C., and UV detection wavelength 214 nm.

### Example 3. Binding Characterization

BIAcore Analysis

The binding activity of the TCR molecule to VLDGLDVLL-HLA-A2 complex was detected using BIAcore T200 real-time analysis system. The anti-streptavidin antibody (GenScript) was added to a coupling buffer (10 mM sodium acetate buffer, pH 4.77), and then the antibody was passed through a CM5 chip pre-activated with EDC and NHS to immobilize the antibody on the surface of the chip. The unreacted activated surface was finally blocked with a solution of ethanolamine in hydrochloric acid to complete the coupling process at a coupling level of about 15,000 RU.

A low concentration of streptavidin flowed over the surface of the antibody-coated chip, then VLDGLDVLL-HLA-A2 complex flowed through the detection channel with another channel being used as a reference channel. 0.05 mM biotin flowed over the chip for 2 min at a flow rate of 10 μL/min, thereby blocking the remaining binding sites for streptavidin. The affinity was determined by single-cycle kinetic analysis. TCR was diluted to several different concentrations with HEPES-EP buffer (10 mM HEPES, 150 mM NaCl, 3 mM EDTA, 0.005% P20, pH 7.4), and flowed over the the surface of the chip in turn at a flow rate of 30 μL/min, with a binding time of 120 s per injection. After the last injection, the chip was placed for dissociation for 600 s. At the end of each round of assay, the chip was regenerated with 10 mM Gly-HCl, pH 1.75. Kinetic parameters were calculated using BIAcore Evaluation software.

The preparation process for the above VLDGLDVLL-HLA-A2 complex is described as follows:

a. Purification

100 ml of E. coli liquid induced to express heavy or light chain was collected, and centrifuged at 8000 g for 10 min at 4° C., and the cells were washed once with 10 ml of PBS, and then vigorously shaken in 5 ml of BugBuster Master Mix Extraction Reagents (Merck) for resuspending the cells. The suspension was incubated for 20 min at room temperature, and then centrifuged at 6000 g for 15 min at 4° C. The supernatant was discarded to collect inclusion bodies.

The above inclusion bodies was resuspended in 5 ml BugBuster Master Mix and incubated vortically at room temperature for 5 min. 30 ml of 10 time-diluted BugBuster was added, mixed, and centrifuged at 6000 g for 15 min at 4° C. The supernatant was discarded, 30 ml of 10 time-diluted BugBuster was added to resuspend the inclusion body, mixed, and centrifuged twice at 6000 g at 4° C. for 15 min. 30 ml of 20 mM Tris-HC1 pH 8.0 was added to resuspend the inclusion bodies, mixed, and centrifuged at 6000 g at 4° C. for 15 min. Finally, inclusion bodies were dissolved in 20 mM Tris-HCl 8M urea, and the purity of inclusion bodies was determined by SDS-PAGE and the concentration was measured by BCA kit.

b. Refolding

Synthesized short peptide VLDGLDVLL (Beijing Saibaisheng Gene Technology Co., Ltd.) were dissolved in DMSO to a concentration of 20 mg/ml. Inclusion bodies of light and heavy chains were solubilized in 8 M urea, 20 mM Tris pH 8.0, 10 mM DTT, and further denatured by adding 3 M guanidine hydrochloride, 10 mM sodium acetate, 10 mM EDTA before refolding. VLDGLDVLL peptide was added to a refolding buffer (0.4 M L-arginine, 100 mM Tris pH 8.3, 2 mM EDTA, 0.5 mM oxidized glutathione, 5 mM reduced glutathione, 0.2 mM PMSF, cooled to 4° C.) at 25 mg/L (final concentration). Then 20 mg/L of light chain and 90 mg/L of heavy chain (final concentration, heavy chain was added in three portions, 8 h/portion) were successively added, and refolded at 4° C. for at least 3 days to completion of refolding, and SDS-PAGE was used to confirm refolding.

c. Purification Upon Refolding

The refolding buffer was replaced with 10 volumes of 20 mM Tris pH 8.0 for dialysis, and the buffer was exchanged for at least two times to substantially reduce the ionic strength of the solution. After dialysis, the protein solution was filtered through a 0.45 μm cellulose acetate filter and loaded onto a HiTrap Q HP (GE, General Electric Company) anion exchange column (5 ml bed volume). The protein was eluted with a linear gradient of 0-400 mM NaCl prepared in 20 mM Tris pH 8.0 using Akta Purifier (GE), and the pMHC was eluted at approximately 250 mM NaCl. Peak fractions were collected and the purity thereof was detected by SDS-PAGE.

d. Biotinylation

Purified pMHC molecules were concentrated in a Millipore ultrafiltration tube, while the buffer was replaced with 20 mM Tris pH 8.0, and then biotinylation reagent 0.05 M Bicine pH 8.3, 10 mM ATP, 10 mM MgOAc, 50 μM D-Biotin, 100 μg/m1 BirA enzyme (GST-BirA) was added. The resulting mixture was incubated at room temperature overnight, and SDS-PAGE was used to detect the completion of biotinylation.

e. Purification of Biotinylated Complex

The biotinylated and labeled pMHC molecules were concentrated to 1 ml in a Millipore ultrafiltration tube. The biotinylated pMHC was purified by gel filtration chromatography. 1 ml of concentrated biotinylated pMHC molecules was loaded on a HiPrep™ 16/60 5200 HR column (GE) pre-equilibrated with filtered PBS using an Akta Purifier (GE) and eluted with PBS at a flow rate of 1 ml/min. The biotinylated pMHC molecules were eluted as a single peak at about 55 ml. The protein-containing fractions were combined and concentrated in a Millipore ultrafiltration tube. The concentration of protein was determined by BCA method (Thermo), protease inhibitor cocktail (Roche) was added and the biotinylated pMHC molecules were dispensed and stored at −80° C.

### Example 4. Generation of High-Affinity Single-Chain TCR

Phage display technology is a means to generate high affinity TCR variant libraries for screening high affinity variants. The TCR phage display and screening method described by Li et al. ((2005) Nature Biotech 23(3): 349-354) was applied to the single-chain TCR template of Example 1. A library of high affinity TCRs was established by mutating CDR regions of the template chain and panned. After several rounds of panning, the phage library can specifically bind to the corresponding antigen, the monoclone was picked and sequence analysis was performed.

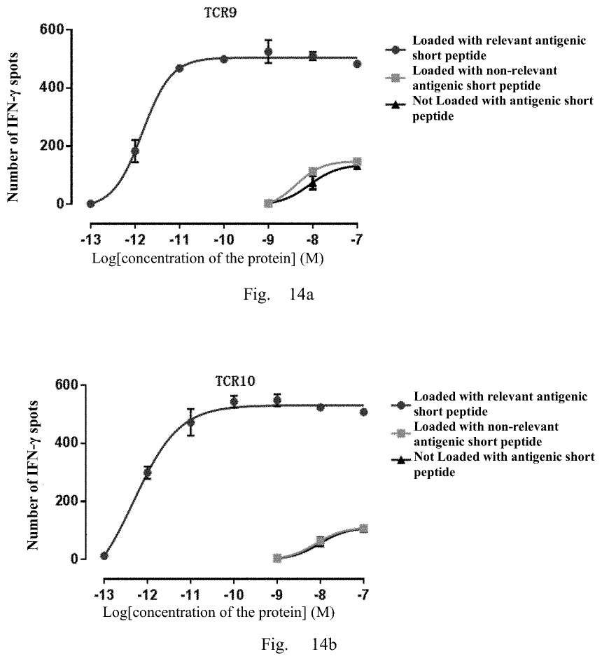

BIAcore method of Example 3 was used to analyze the interaction between a TCR molecule and VLDGLDVLL-HLA-A2 complex, and a high affinity TCR with affinity and/or binding half-life of at least 2 times that of the wild-type TCR was screened out, that is, the dissociation equilibrium constant K_{D} of the screened high affinity TCR for binding VLDGLDVLL-HLA-A2 complex is less than or equal to one-half of the dissociation equilibrium constant K_{D} of the wild type TCR for binding SLLMWITQC-HLA-A2 complex, and the results are shown in Table 3 below. K_{D} value of the interaction between the reference TCR and VLDGLDVLL-HLA-A2 complex was detected to be 11 μM by using the above method, and the interaction curve is shown in FIG. 12, that is, K_{D} value of the wild type TCR interacting with VLDGLDVLL-HLA-A2 complex is also 11 μM (1.1E-05M).

Specifically, when using the numbering shown in SEQ ID NO: 1, an amino acid at one or more of the following sites in the α chain variable domain of these high-affinity TCR mutants mutated: 30S, 32S, 50I, 51Y, 52S, 53N, 92A, 93R, 94T, 95Y, 96T, 97G, 98N, 99Q and/or when using the numbering shown in SEQ ID NO: 2, an amino acid at one or more of the following sites in the β chain variable domain of these high-affinity TCR mutants mutated: 51N, 52E, 53A, 54Q, 95S, 96S, 97Q, 98K, 99F.

More specifically, when using the numbering shown in SEQ ID NO: 1, the α chain variable domains of these high-affinity TCRs comprise one or more amino acid residues selected from the group consisting of 30T or 30A; 32A; 50Q, SOL or 50T; 51V; 52M, 52V, 52Q; 53P or 53D; 92V; 93L; 94S; 95W; 96K, 96A, 96R, 96L, 96Q, 96F or 97S; 98T; and 99R or 99G; and/or when using the numbering shown in SEQ ID NO: 2, the β chain variable domains of these high-affinity TCRs comprise one or more amino acid residues selected from the group consisting of 51D or 51G; 52S or 52R; 531 or 53S; 54E; 95N; 96A, 96P, 96N, 96K, 96Q, 96T, 96M or 96R; 97S, 97G or 97T; 98G, 98P or 98L; 99V or 99L.

The specific amino acid sequences of α chain variable domains (SEQ ID NOs: 9, 10, 11, 12, 13, 14, 15, 16, 17, 18, 19, 20, 21, 22, 23, 24, 25, 26, 27, 28, 29, 30, 31, 32, 33 and 34) and β chain variable domains (SEQ ID NO: 35, 36, 37, 38, 39, 40, 41, 42, 43, 44 45, 46, 47, 48, 49, 50, 51, 52 and 53) of the high-affinity single-chain TCRs are shown in FIGS. 5(1)-(26) and FIGS. 6(1)-(18), respectively.

TABLE 3

| | | |
|---|---|---|
| | Single chain TCR variable | |
| Single chain | domain (SEQ ID NO.) | K_{D} |

| TCR No. | α | β | (M) |
|---|---|---|---|
| s-1 | 9 | 4 | 8.29E−08 |
| s-2 | 10 | 4 | 1.43E−06 |
| s-3 | 11 | 4 | 6.85E−08 |
| s-4 | 12 | 4 | 1.02E−08 |
| s-5 | 13 | 4 | 3.35E−07 |
| s-6 | 14 | 4 | 2.85E−06 |
| s-7 | 15 | 4 | 2.60E−06 |
| s-8 | 3 | 35 | 5.74E−06 |
| s-9 | 3 | 36 | 7.66E−06 |
| s-10 | 3 | 37 | 4.08E−06 |
| s-11 | 3 | 38 | 1.46E−05 |
| s-12 | 3 | 39 | 2.46E−06 |
| s-13 | 3 | 40 | 3.90E−05 |
| s-14 | 3 | 41 | 2.04E−06 |
| s-15 | 3 | 42 | 1.15E−07 |
| s-16 | 3 | 43 | 6.07E−05 |
| s-17 | 3 | 44 | 2.22E−07 |
| s-18 | 16 | 45 | 1.15E−09 |
| s-19 | 17 | 46 | 1.30E−09 |
| s-20 | 18 | 47 | 7.53E−10 |
| s-21 | 19 | 48 | 1.01E−09 |
| s-22 | 20 | 49 | 6.92E−10 |
| s-23 | 21 | 45 | 1.07E−09 |
| s-24 | 22 | 47 | 1.04E−09 |
| s-25 | 23 | 50 | 8.76E−10 |
| s-26 | 24 | 45 | 1.15E−09 |
| s-27 | 25 | 45 | 1.36E−09 |
| s-28 | 26 | 51 | 9.78E−10 |
| s-29 | 27 | 50 | 2.79E−10 |
| s-30 | 28 | 50 | 2.87E−10 |
| s-31 | 29 | 52 | 4.26E−10 |
| s-32 | 30 | 49 | 5.55E−10 |
| s-33 | 31 | 50 | 5.85E−10 |
| s-34 | 32 | 45 | 6.35E−10 |
| s-35 | 33 | 47 | 4.70E−10 |
| s-36 | 34 | 49 | 2.48E−10 |

### Example 5. Generation of High-Affinity αβ Heterodimeric TCR

The mutations in CDR regions of the high-affinity single-chain TCRs screened in Example 4 were introduced into the corresponding sites of the variable domain of the αβ heterodimeric TCR, and its affinity for VLDGLDVLL-HLA-A2 complex was detected by BIAcore. The mutated sites of high-affinity can be introduced in the above CDR regions by a method of site-directed mutagenesis well known to a skilled person in the art. The amino acid sequences of α chain and β chain variable domain of the above wild type TCR are shown in FIGS. 1a (SEQ ID NO: 1) and 1b (SEQ ID NO: 2), respectively.

It should be noted that in order to obtain a more stable soluble TCR for easier evaluation of the binding affinity and/or binding half-life between the TCR and VLDGLDVLL-HLA-A0201 complex, the αβ heterodimeric TCR may be such a TCR in which a cysteine residue is respectively introduced into α and β chain constant domain to form an artificial interchain disulfide bond. In this example, the amino acid sequences of TCR α and β chains after introducing a cysteine residue are shown in FIG. 8a (SEQ ID NO: 55) and 8b (SEQ ID NO: 56), and the introduced cysteine residues are indicated by bold letters.

According to standard methods described in Molecular Cloning a Laboratory Manual (3rd edition, Sambrook and Russell), genes of extracellular sequences of the TCR α and β chains to be expressed are synthesized and inserted into an expression vector pET28a+ (Novagene), in which the upstream and downstream cloning sites are NcoI and NotI, respectively. Mutations in the CDR regions are introduced by overlap PCR well known to a skilled person in the art. The inserted fragment was sequenced to confirm that it was correct.

### Example 6. Expression, Refolding and Purification of αβ Heterodimeric TCR

Expression vectors for TCR α and β chain were transformed into the expression bacteria BL21 (DE3) by chemical transformation, respectively. The bacteria were grown in LB medium and induced with a final concentration of 0.5 mM IPTG at OD₆₀₀=0.6. The inclusion bodies formed after the TCR α and β chains were expressed were extracted by BugBuster Mix (Novagene) and repeatedly washed with BugBuster solution. The inclusion bodies were finally dissolved in 6 M guanidine hydrochloride, 10 mM dithiothreitol (DTT), 10 mM ethylenediaminetetraacetic acid (EDTA) and 20 mM Tris (pH 8.1).

The dissolved TCR α and β chains were rapidly mixed in 5 M urea, 0.4 M arginine, 20 mM Tris (pH 8.1), 3.7 mM cystamine, and 6.6 mM β-mercapoethylamine (4° C.) at a mass ratio of 1:1. The final concentration is 60 mg/mL. After mixing, the solution was dialyzed against 10 volumes of deionized water (4° C.), and after 12 hours, deionized water was exchanged with a buffer (20 mM Tris, pH 8.0) and dialysis was continued at 4° C. for 12 hours. After completion of the dialysis, the solution was filtered through a 0.45 μM filter and purified through an anion exchange column (HiTrap Q HP, 5 ml, GE Healthcare). The elution peak of TCR containing successfully refolded α and β dimers was confirmed by SDS-PAGE gel. The TCR was then further purified by gel filtration chromatography (HiPrep 16/60, Sephacryl S-100 HR, GE Healthcare). The purity of the purified TCR was determined by SDS-PAGE to be greater than 90%, and the concentration thereof was determined by BCA method.

### Example 7. Results of BIAcore Analysis

The affinity of the αβ heterodimeric TCR, in which a high affinity CDR region was introduced, for VLDGLDVLL-HLA-A2 complex was detected by using the method described in Example 3.

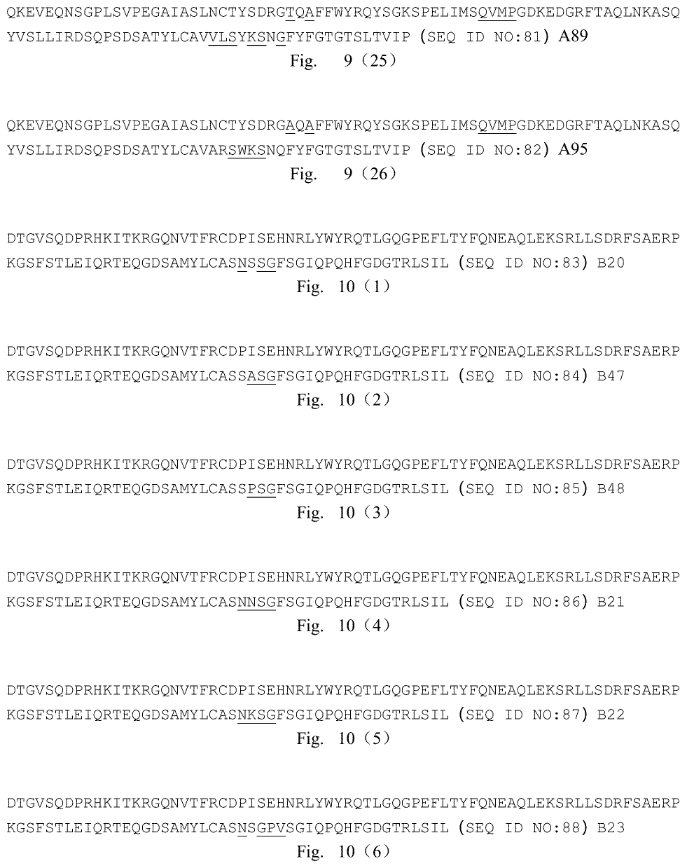

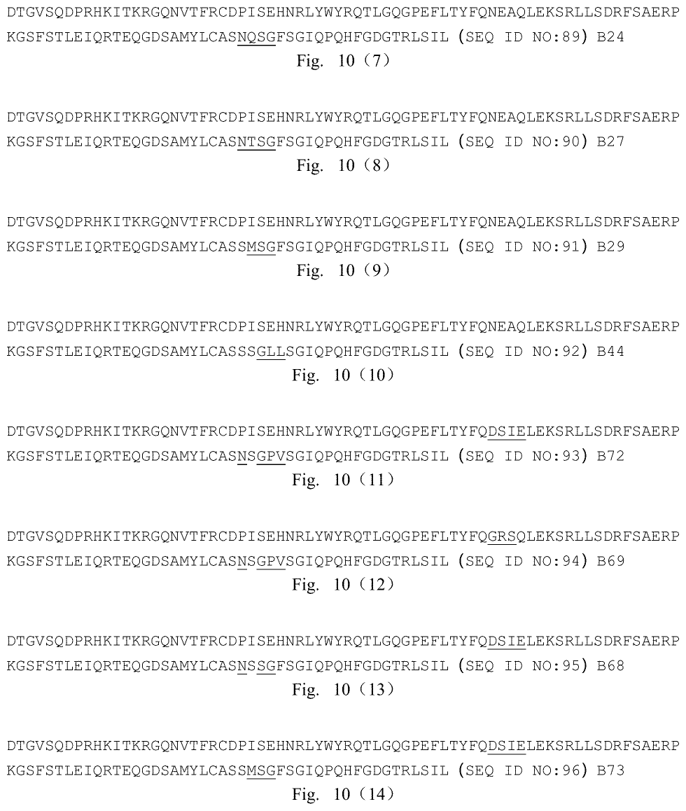

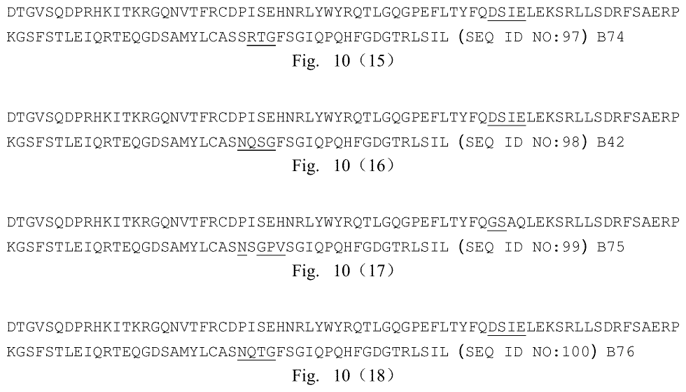

The CDR regions selected from the high-affinity single-chain TCR α and β chain were transferred into the corresponding positions of the wild-type TCR α chain variable domain SEQ ID NO: 1 and β chain variable domain SEQ ID NO: 2, respectively, to form an αβ heterodimeric TCR. The amino acid sequences of resulting new TCR α and β chain variable domains are shown in FIGS. 9(1)-(26) and FIGS. 10(1)-(18), respectively. Since the CDR regions of a TCR molecule determine their affinity for the corresponding pMHC complex, a skilled person in the art can anticipate that an a(3 heterodimeric TCR, in which a high affinity mutation site is introduced also has a high affinity for VLDGLDVLL-HLA-A2 complex. The expression vector was constructed by the method described in Example 5, the above-mentioned αβ heterodimeric TCR with a high-affinity mutation being introduced was expressed, refolded and purified by the method described in Example 6, and then the affinity of the TCR for VLDGLDVLL-HLA-A2 complex is determined by BIAcore T200, as shown in Table 4 below.

TABLE 4

| | TCR variable domain | | |
|---|---|---|---|
| | (SEQ ID NO) | | K_{D} |
| TCR No. | α | β | (M) |
| 1 | 57 | 2 | 4.01E−08 |
| 2 | 58 | 2 | 2.27E−07 |
| 3 | 59 | 2 | 2.98E−07 |
| 4 | 60 | 2 | 3.75E−07 |
| 5 | 61 | 2 | 1.15E−06 |
| 6 | 62 | 2 | 5.604E−07 |
| 7 | 63 | 2 | 5.674E−07 |
| 8 | 1 | 83 | 2.08E−06 |
| 9 | 1 | 84 | 1.87E−06 |
| 10 | 1 | 85 | 1.07E−06 |
| 11 | 1 | 86 | 4.63E−06 |
| 12 | 1 | 87 | 1.53E−06 |
| 13 | 1 | 88 | 1.24E−06 |
| 14 | 1 | 89 | 1.63E−06 |
| 15 | 1 | 90 | 1.17E−06 |
| 16 | 1 | 91 | 3.03E−06 |
| 17 | 1 | 92 | 3.52E−06 |
| 18 | 64 | 93 | 5.51E−10 |
| 19 | 65 | 94 | 2.82E−10 |
| 20 | 66 | 95 | 1.73E−10 |
| 21 | 67 | 96 | 4.38E−10 |
| 22 | 68 | 97 | 1.57E−10 |
| 23 | 69 | 93 | 5.01E−10 |
| 24 | 70 | 95 | 3.46E−10 |
| 25 | 71 | 98 | 5.41E−10 |
| 26 | 72 | 93 | 5.69E−10 |
| 27 | 73 | 93 | 8.43E−10 |
| 28 | 74 | 99 | 5.82E−10 |
| 29 | 75 | 98 | 1.80E−10 |
| 30 | 76 | 98 | 1.57E−10 |
| 31 | 77 | 100 | 9.33E−09 |
| 32 | 78 | 97 | 2.28E−10 |
| 33 | 79 | 98 | 1.84E−10 |
| 34 | 80 | 93 | 2.45E−10 |
| 35 | 81 | 95 | 7.645E−11 |
| 36 | 82 | 97 | 6.761E−11 |

As can be seen from Table 4 above, the αβ heterodimeric TCR with mutation sites introduced into CDR regions maintains high affinity for VLDGLDVLL-HLA-A2 complex. The affinity of the heterodimeric TCR for VLDGLDVLL-HLA-A2 complex is at least 2 times of that of the wild-type TCR.

### Example 8. Expression, Refolding and Purification of Fusions of Anti-CD3 Antibodies with High-Affinity Single-Chain TCR

The high-affinity single-chain TCR molecule of the present invention is fused with a single-chain molecule (scFv) of an anti-CD3 antibody to construct a fusion molecule. Primers were designed by overlapping PCR, and the genes of anti-CD3 antibodies and high-affinity single-chain TCR molecule were ligated. The intermediate linker was designed as GGGGS, and the gene fragment of the fusion molecule had restriction enzyme sites NcoI and NotI. The PCR amplification product was digested with NcoI and NotI and ligated with pET28a vector digested with NcoI and NotI. The ligation product was transformed into E. coli DH5α competent cells, and plated on LB plate containing kanamycin and inverted and cultured overnight at 37° C. Positive clones were picked for PCR screening, and the positive recombinants were sequenced to determine the correct sequence. The recombinant plasmids were extracted and transformed into E. coli BL21 (DE3) competent cells for expression.

Expression of Fusion Protein

The expression plasmid containing the gene of interest was transformed into E. coli strain BL21 (DE3), plated on a LB plate (kanamycin, 50 μg/ml) and cultured at 37° C. overnight. On the next day, the clones were picked and inoculated into 10 ml of LB liquid medium (kanamycin, 50 μg/ml), cultured for 2-3 h, then inoculated to 1 L of LB medium (kanamycin, 50 μg/ml) at a volume ratio of 1:100, cultured until the OD₆₀₀ was 0.5-0.8, and then induced to express the protein of interest using IPTG at a final concentration of 0.5 mM. 4 hours after induction, the cells were harvested by centrifugation at 6000 rpm for 10 min. The cells were washed once in PBS buffer, and dispensed. Cells corresponding to 200 ml of the bacterial culture were taken and lysed with 5 ml of BugBuster Master Mix (Novagen), and the inclusion bodies were collected by centrifugation at 6000 g for 15 minutes. The inclusion bodies were washed for 4 times with detergent to remove cell debris and membrane components. The inclusion bodies are then washed with a buffer such as PBS to remove detergent and salt. Finally, the inclusion bodies were dissolved in Tris buffer solution containing 8 M urea, the concentration of inclusion bodies was measured, and inclusion bodies were dispensed and cryopreserved at −80° C.

Refolding of Fusion Proteins

About 10 mg of inclusion bodies were taken from a −80° C. ultra-low temperature freezer and thawed, and dithiothreitol (DTT) was added to a final concentration of 10 mM, and incubated at 37° C. for 30 min to 1 hour to ensure complete opening of the disulfide bond. The solution of inclusion body sample was then added dropwise into 200 ml of 4° C. pre-cooled refolding buffer (100 mM Tris pH 8.1, 400 mM L-arginine, 2 mM EDTA, 5 M urea, 6.5 mM β-mercapthoethylamine, 1.87 mM Cystamine), respectively, and stirred slowly at 4° C. for about 30 minutes. The refolding solution was dialyzed against 8 volumes of pre-cooled H2O for 16-20 hours. It was further dialyzed twice with 8 volumes of 10 mM Tris pH 8.0, and dialysis was continued at 4° C. for about 8 hours. After dialysis, the sample was filtered and subjected to the following purification process.

First Step Purification of Fusion Protein

The dialyzed and refolded material (in 10 mM Tris pH 8.0) was eluted on a POROS HQ/20 anion exchange chromatography prepacked column (Applied Biosystems) with a gradient of 0-600 mM NaCl using AKTA Purifier (GE Healthcare). Each component was analyzed by Coomassie brilliant blue stained SDS-PAGE and then combined.

Second Step Purification of the Fusion Protein

The purified and combined sample solution from the first step was concentrated for purification in this step, and the fusion protein was purified by Superdex 75 10/300 GL gel filtration chromatography prepacked column (GE Healthcare) pre-equilibrated in PBS buffer. The components of the peaks were analyzed by Coomassie Brilliant Blue-stained SDS-PAGE and then combined.

### Example 9. Expression, Refolding and Purification of Fusions of Anti-CD3 Antibody with High-Affinity αβ Heterodimeric TCR

A fusion molecule was prepared by fusing an anti-CD3 single-chain antibody (scFv) with an αβ heterodimeric TCR. The anti-CD3 scFv was fused with β chain of the TCR, and the TCR β chain may comprise β chain variable domain of any of the above high-affinity αβ heterodimeric TCRs, and the TCR α chain of the fusion molecule may comprise α chain variable domain of any of the above high-affinity αβ heterodimeric TCR.

Construction of Expression Vector for Fusion Molecule

1. Construction of Expression Vector for α Chain

The target gene carrying α chain of the αβ heterodimeric TCR was digested with NcoI and NotI, and ligated with pET28a vector digested with NcoI and NotI. The ligation product was transformed into E. coli DH5α, plated on a LB plate containing kanamycin, and inverted and cultured overnight at 37° C. Positive clones were picked for PCR screening, and the positive recombinants were sequenced to determine the correct sequence. The recombinant plasmids were extracted and transformed into E. coli Tuner (DE3) for expression.

2. Construction of Expression Vector for Anti-CD3 (scFv)-β Chain

Primers were designed by overlapping PCR to connect genes of the anti-CD3 scFv and high-affinity heterodimeric TCRβ chain. The anti-CD3 scFv can be connected to the N-terminal or C terminal of the TCRβ chain. In Example 11 of the present invention, the TCRs, in which anti-CD3 scFv was connected to the N terminal are TCR9, TCR10 and TCR11; the TCRs, in which anti-CD3 scFv was connected to the C terminal are TCR12, TCR13 and TCR14. The intermediate linker was GGGGS, and the gene fragment of the fusion protein of anti-CD3 scFv and the high-affinity heterodimeric TCRβ chain had the restriction endonuclease sites NcoI (CCATGG) and NotI (GCGGCCGC). The PCR amplification product was digested with NcoI and NotI and ligated with pET28a vector digested with NcoI and NotI. The ligation product was transformed into E. coli DH5α competent cells, plated on a kanamycin-containing LB plate, and inverted and cultured overnight at 37° C. Positive clones were picked for PCR screening, and the positive recombinants were sequenced to determine the correct sequence. The recombinant plasmids were extracted and transformed into E. coli Tuner (DE3) competent cells for expression.

Expression, Refolding and Purification of Fusion Protein

The expression plasmids were separately transformed into E. coli Tuner (DE3) competent cells, plated on LB plates (kanamycin 50 μg/mL) and cultured overnight at 37° C. On the next day, clones were picked and inoculated into 10 mL LB liquid medium (kanamycin 50 μg/mL) for 2-3 h, and inoculated into 1 L LB medium at a volume ratio of 1:100, the culture was continued until the OD600 was 0.5-0.8, and a final concentration of 1 mM IPTG was added to induce expression of the protein of interest. After 4 hours, cells were harvested by centrifugation at 6000 rpm for 10 mins. The cells were washed once in PBS buffer and were dispensed, and cells corresponding to 200 mL of the bacterial culture were taken and lysed with 5 mL of BugBuster Master Mix (Merck), inclusion bodies were collected by centrifugation at 6000 g for 15 min and then washed with detergent for 4 times to remove cell debris and membrane components. The inclusion bodies were then washed with a buffer such as PBS to remove detergent and salt. Finally, the inclusion bodies were dissolved in 6M guanidine hydrochloride, 10 mM dithiothreitol (DTT), 10 mM ethylenediaminetetraacetic acid (EDTA), 20 mM Tris, pH 8.1 buffer solution, and the concentration of inclusion bodies was determined. The inclusion bodies were dispensed and cryopreserved at −80° C.

The dissolved TCRα chain and anti-CD3 (scFv)-β chain were rapidly mixed in a mass ratio of 2: 5 in 5 M urea (urea), 0.4 M L-arginine (L-arginine), 20 mM Tris pH 8.1, 3.7 mM cystamine, and 6.6 mM β-mercapoethylamine (4° C.), and the final concentrations of α chain and anti-CD3 (scFv)-β chain were 0.1 mg/mL, 0.25 mg/mL, respectively.

After mixing, the solution was dialyzed against 10 volumes of deionized water (4° C.), and after 12 hours, deionized water was exchanged with buffer (10 mM Tris, pH 8.0) for another 12 hours at 4° C. After completion of dialysis, the solution was filtered through a 0.45 μM filter and purified by an anion exchange column (HiTrap Q HP 5 ml, GE healthcare). The eluted peaks containing the reconstituted TCR α chain and anti-CD3 (scFv)-β chain dimer TCR were confirmed by SDS-PAGE gel. The TCR fusion molecule was then purified by size exclusion chromatography (S-100 16/60, GE healthcare) and further purified by an anion exchange column (HiTrap Q HP 5 ml, GE healthcare). The purity of the purified TCR fusion molecule was determined by SDS-PAGE to be greater than 90%, and the concentration was determined by BCA method.

### Example 10. Activation Function Assay of Effector Cells Transfected with High Affinity TCR of the Present Invention

This example demonstrates that effector cells transfected with the high affinity TCR of the present invention have a good specific activation effect on target cells.

The function and specificity of the high affinity TCR of the present invention in cells were examined by ELISPOT assay. Methods for detecting cellular function using ELISPOT assays are well known to a skilled person in the art. In the IFN-γ ELISPOT assay of this example, CD8⁺ T cells isolated from the blood of healthy volunteers and transfected with TCR using lentivirus were used as effector cells, and labeled as TCR1 (α chain variable domain SEQ ID NO: 10, β chain variable domain SEQ ID NO: 2), TCR2 (α chain variable domain SEQ ID NO: 11, β chain variable domain SEQ ID NO: 2), TCR3 (α chain variable domain SEQ ID NO: 12, β chain variable domain SEQ ID NO: 2) and TCR4 (α chain variable domain SEQ ID NO: 13, β chain variable domain SEQ ID NO: 2) as the first group; TCR5 (α chain variable domain SEQ ID NO: 1, β chain variable domain SEQ ID NO: 39), TCR6 (α chain variable domain SEQ ID NO: 1, β chain variable domain SEQ ID NO: 40), TCR7 (α chain variable domain SEQ ID NO: 1, β chain variable domain SEQ ID NO: 43) and TCR8 (α chain variable domain SEQ ID NO: 1, β chain variable domain SEQ ID NO: 44) as the second group, respectively; and effector cells in the control group were labeled as WT-TCR (transfected with wild-type TCR). The target cell lines were A375(A2/PRAME⁺), K562-A2(PRAME⁺) and Mel526(A2/PRAME⁺) cells as the control. Cell lines with mismatched genotypes or not expressing relevant antigens K562-A2(PRAME⁺) and SW620(A2/PRAME⁻) were used as controls.

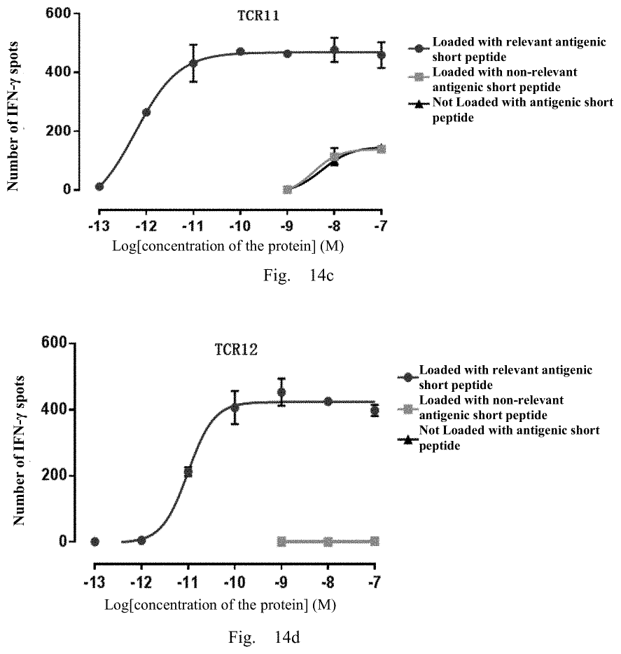

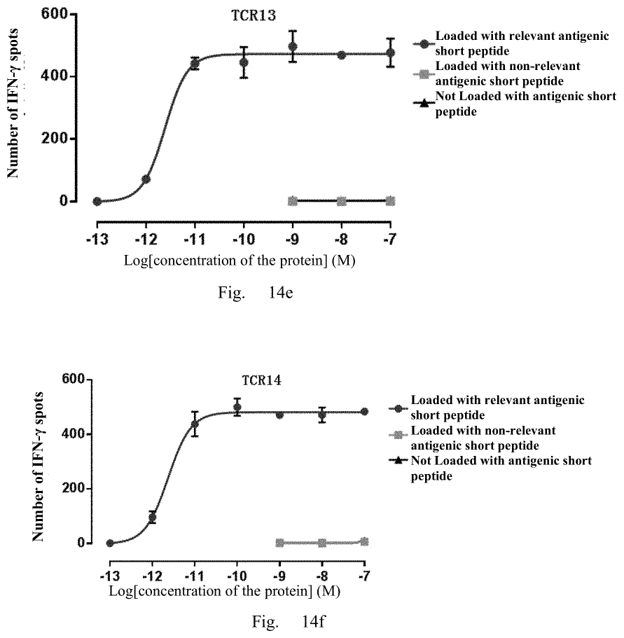

Firstly, a ELISPOT plate was prepared. The ELISPOT plate was activated and coated with ethanol overnight at 4° C. On the first day of the experiment, the coating solution was removed, and the plate was washed, blocked and incubated at room temperature for 2 hrs, and the blocking solution was removed. Components of the assay were added to the ELISPOT plate in the following order: the medium for adjusting effector cells to 1×10⁵ cells/ml, and the medium for adjusting each target cell line to 2×10⁵ cells/ml. After homogeneously mixing, 100 μL of target cell line 2×10⁵ cells/ml (i.e., 20,000 cells/well) and 100 μL of effector cells 1×10⁵ cells/ml (i.e., 10,000 cells/well) were added to the corresponding wells in duplicate, and incubate overnight (37° C., 5% CO₂). On the second day of the experiment, the plate was washed, subjected to a secondary detection and development, and dried, and the spots formed on the film were counted using an immunospot plate reader (ELISPOT READER system; AID20 company). Experimental results are shown in FIG. 13a (the first group) and FIG. 13b (the second group), in which the effector cells transfected with the high affinity TCR of the present invention exhibit excellent specific activation effects on target cells.

### Example 11. Functional Assay of Fusion Protein of High Affinity TCR of the Present Invention and anti-CD3 Antibody

This example demonstrates that the fusion protein of the high affinity TCR of the present invention and anti-CD3 antibody is capable of redirecting effector cells and exhibits good activation effects.

Methods for detecting cellular function using ELISPOT assays are well known to a skilled person in the art. The effector cells used in IFN-γ ELISPOT assay of this example were CD8+ T cells isolated from the blood of healthy volunteers. The target cells were T2 cells loaded with the relevant antigenic short peptides of the present invention, and T2 cells not loaded with antigenic short peptides or loaded with other unrelevant antigenic short peptides were used as controls. The high affinity TCR of the present invention was randomly selected and the fusion protein was prepared as described in Example 9. In particular, α and β chains of the selected high affinity TCR were as follows are listed as follows: TCR9 (α chain SEQ ID NO: 64, β chain SEQ ID NO: 93); TCR10 (α chain SEQ ID NO: 65, β chain SEQ ID NO: 94); TCR11 (α chain SEQ ID NO: 67, β chain SEQ ID NO: 96); TCR12 (α chain SEQ ID NO: 66, β chain SEQ ID NO: 95); TCR13 (α chain SEQ ID NO: 69, β chain SEQ ID NO: 93); TCR14 (α chain SEQ ID NO: 70, β chain SEQ ID NO: 95).

Firstly, a ELISPOT plate was prepared. ELISPOT plate was activated and coated with ethanol, and incubated overnight at 4° C. On the first day of the experiment, the coating solution was removed, and the plate was washed, blocked and incubated at room temperature for two hours, and the blocking solution was removed. Components of the assay were added to the ELISPOT plate in the following order: medium for adjusting CD8+ T cells to 8×10⁴ cells/ml, medium for adjusting each target cell line to 4×10⁵ cells/ml, medium for diluting the polypeptide to a concentration of 0.04 μM, and medium for diluting the fusion protein to a concentration of 0.04 μM (serially diluted by 10 times). After homogeneously mixing, 50 μL of fusion protein dilution, 50 μL of target cell line 4×10⁵ cells/ml (i.e., 20,000 cells/well) and 50 μL of effector cells 8×10⁴ cells/ml (i.e., 4,000 effect cells/well) were taken and added to the corresponding wells in duplicate. The plate was incubated overnight (37° C., 5% CO₂). On the second day of the experiment, the plate was washed and subjected to the secondary detection and color development. The plate was dried, and the spots formed on the film were counted using an immunospot plate reader (ELISPOT READER system; AID20 company). Experiment results are shown in FIGS. 14a, 14b, 14c, 14d, 14e and 14f, and the fusion protein of the high affinity TCR of the present invention and anti-CD3 antibody is capable of redirecting effector cells and exhibts good activating effects.

All documents mentioned in the present application are hereby incorporated by reference in their entireties, as if each is incorporated by reference. In addition, it should be understood that after reading the teachings of the present invention described above, a skilled person in the art can make various changes or modifications of the invention, and these equivalent forms also fall into the scope as defined by the appended claims of the present application.

1-60. (canceled)

61. A T cell receptor (TCR), which has an activity of binding to VLDGLDVLL-HLA-A0201 complex, the T cell receptor comprises a TCR α chain variable domain and a TCR β chain variable domain, the α chain variable domain of the TCR comprises an amino acid sequence having at least 90% of sequence homology with the amino acid sequence shown in SEQ ID NO: 1, and the β chain variable domain of the TCR comprises an amino acid sequence having at least 90% of sequence homology with the amino acid sequence shown in SEQ ID NO: 2, the TCRα chain variable domain comprises three CDR regions, and the reference sequences of the three CDR regions of the TCR α chain variable domain are listed as follows,
CDR1α:
DRGSQS
CDR2α:
IYSNGD
CDR3α:
AVARTYTGNQFY,
the TCR β chain variable domain comprises three CDR regions, and the reference sequences of the three CDR regions of the TCR β chain variable domain are listed as follows,
CDR1β:
SEHNR
CDR2β:
FQNEAQ
CDR3β:
ASSSQKFSGIQPQH,
and the TCRα chain variable domain contains at least one of the following mutations:

| Residue before mutation | Residue after mutation |
|---|---|
| S at position 4 of CDR1α | T or A |
| S at position 6 of CDR1α | A |
| I at position 1 of CDR2α | Q or L or T |
| Y at position 2 of CDR2α | V |
| S at position 3 of CDR2α | M or V or Q |
| N at position 4 of CDR2α | P or D |
| A at position 3 of CDR3α | V |
| R at position 4 of CDR3α | L |
| T at position 5 of CDR3α | S |
| Y at position 6 of CDR3α | W |
| T at position 7 of CDR3α | K or A or R or L or Q or F |
| G at position 8 of CDR3α | S |
| N at position 9 of CDR3α | T |
| Q at position 10 of CDR3α | G or R |

and/or, the TCR β chain variable domain contains at least one of the following mutations:

| Residue before mutation | Residue after mutation |
|---|---|
| N at position 3 of CDR2β | D or G |
| E at position 4 of CDR2β | S or R |
| A at position 5 of CDR2β | I or S |
| Q at position 6 of CDR2β | E |
| S at position 3 of CDR3β | N |
| S at position 4 of CDR3β | A or P or N or K or Q or T or M or R |
| Q at position 5 of CDR3β | S or G or T |
| K at position 6 of CDR3β | P or G or L |
| F at position 7 of CDR3β | V or L. |

62. The TCR of claim 61, wherein the affinity of the TCR for VLDGLDVLL-HLA-A0201 complex is at least 2 times of that of the wild type TCR.

63. The TCR of claim 61, wherein the TCR comprises a TCR α chain variable domain and a TCR β chain variable domain, the TCR α chain variable domain comprises CDR1α, CDR2α and CDR3α, and the sequence of CDR3α is selected from the group consisting of:
AVARTYTGNQFY, AVARSWKSNQFY, AVARSWASNQFY, AVARTYRSTGFY and AVARTYKSTGFY; and/or the amino acid sequence of CDR1α is selected from the group consisting of:
DRGSQS, DRGTQA, DRGAQA and DRGSQA; and/or the amino acid sequence of CDR2α is selected from the group consisting of:
IYSNGD, QVMPGD, QVVPGD and LVQPGD.

64. The TCR of claim 61, wherein the TCR comprises a TCRα chain variable domain and a TCRβ chain variable domain, and the TCRβ chain comprises CDR1β, CDR2β and CDR3β, wherein the amino acid sequence of CDR1β is: SEHNR; and/or the amino acid sequence of CDR2β is selected from the group consisting of:
FQNEAQ, FQDSIE and FQGRSQ; and/or the amino acid sequence of CDR3β is selected from the group consisting of:
ASSSQKFSGIQPQH,
ASNSGPVSGIQPQH,
ASNQSGFSGIQPQH,
ASSMSGFSGIQPQH
and
ASSSGLLSGIQPQH.

65. The TCR of claim 61, wherein the TCR has CDRs selected from the group consisting of:

| TCR No. | CDR1α | CDR2α | CDR3α | CDR1β | CDR2β | CDR3β |
|---|---|---|---|---|---|---|
| 1 | DRGSQS | IYSNGD | AVARSWKSNQFY | SEHNR | FQNEAQ | ASSSQKFSGIQPQH |
| 2 | DRGSQS | IYSNGD | AVARTYLSNRFY | SEHNR | FQNEAQ | ASSSQKFSGIQPQH |
| 3 | DRGSQS | IYSNGD | AVARTWASNQFY | SEHNR | FQNEAQ | ASSSQKFSGIQPQH |
| 4 | DRGSQS | IYSNGD | AVARSYQSNQFY | SEHNR | FQNEAQ | ASSSQKFSGIQPQH |
| 5 | DRGSQS | IYSNGD | AVARSWFSNQFY | SEHNR | FQNEAQ | ASSSQKFSGIQPQH |
| 6 | DRGSQS | IYSNGD | AVARTYKSNRFY | SEHNR | FQNEAQ | ASSSQKFSGIQPQH |
| 7 | DRGSQS | IYSNGD | AVARTYKSNQFY | SEHNR | FQNEAQ | ASSSQKFSGIQPQH |
| 8 | DRGSQS | IYSNGD | AVARTYTGNQFY | SEHNR | FQNEAQ | ASNSSGFSGIQPQH |
| 9 | DRGSQS | IYSNGD | AVARTYTGNQFY | SEHNR | FQNEAQ | ASSASGFSGIQPQH |
| 10 | DRGSQS | IYSNGD | AVARTYTGNQFY | SEHNR | FQNEAQ | ASSPSGFSGIQPQH |
| 11 | DRGSQS | IYSNGD | AVARTYTGNQFY | SEHNR | FQNEAQ | ASNNSGFSGIQPQH |
| 12 | DRGSQS | IYSNGD | AVARTYTGNQFY | SEHNR | FQNEAQ | ASNKSGFSGIQPQH |
| 13 | DRGSQS | IYSNGD | AVARTYTGNQFY | SEHNR | FQNEAQ | ASNSGPVSGIQPQH |
| 14 | DRGSQS | IYSNGD | AVARTYTGNQFY | SEHNR | FQNEAQ | ASNQSGFSGIQPQH |
| 15 | DRGSQS | IYSNGD | AVARTYTGNQFY | SEHNR | FQNEAQ | ASNTSGFSGIQPQH |
| 16 | DRGSQS | IYSNGD | AVARTYTGNQFY | SEHNR | FQNEAQ | ASSMSGFSGIQPQH |
| 17 | DRGSQS | IYSNGD | AVARTYTGNQFY | SEHNR | FQNEAQ | ASSSGLLSGIQPQH |
| 18 | DRGSQS | QVMPGD | AVARSYKSNQFY | SEHNR | FQDSIE | ASNSGPVSGIQPQH |
| 19 | DRGSQS | QVVPGD | AVARTYKSTGFY | SEHNR | FQGRSQ | ASNSGPVSGIQPQH |
| 20 | DRGTQA | QVMPGD | AVARTYKSNGFY | SEHNR | FQDSIE | ASNSSGFSGIQPQH |
| 21 | DRGSQS | QVMPGD | AVARSWASNQFY | SEHNR | FQDSIE | ASSMSGFSGIQPQH |
| 22 | DRGSQS | QVMPGD | AVARSWKSNQFY | SEHNR | FQDSIE | ASSRTGFSGIQPQH |
| 23 | DRGSQA | QVMPGD | AVARTYRSTGFY | SEHNR | FQDSIE | ASNSGPVSGIQPQH |
| 24 | DRGTQA | QVMPGD | AVARTYKSTGFY | SEHNR | FQDSIE | ASNSSGFSGIQPQH |
| 25 | DRGSQS | QVMPGD | AVARTWASNQFY | SEHNR | FQDSIE | ASNQSGFSGIQPQH |
| 26 | DRGTQA | LVQPGD | AVARTYKSTGFY | SEHNR | FQDSIE | ASNSGPVSGIQPQH |
| 27 | DRGSQA | QVVPGD | AVARTWASNQFY | SEHNR | FQDSIE | ASNSGPVSGIQPQH |
| 28 | DRGSQS | LVQPGD | AVARTYKSTGFY | SEHNR | FQGSAQ | ASNSGPVSGIQPQH |
| 29 | DRGAQA | QVMPGD | AVARSWASNQFY | SEHNR | FQDSIE | ASNQSGFSGIQPQH |
| 30 | DRGTQA | QVVPGD | AVARSWKSNQFY | SEHNR | FQDSIE | ASNQSGFSGIQPQH |
| 31 | DRGSQA | QVMPGD | AVARSWTSNQFY | SEHNR | FQDSIE | ASNQTGFSGIQPQH |
| 32 | DRGAQA | IYSNGD | AVARSYKSNQFY | SEHNR | FQDSIE | ASSRTGFSGIQPQH |
| 33 | DRGAQA | TVQDGD | AVARSWKSNQFY | SEHNR | FQDSIE | ASNQSGFSGIQPQH |
| 34 | DRGSQA | IYSNGD | AVARSWKSNQFY | SEHNR | FQDSIE | ASNSGPVSGIQPQH |
| 35 | DRGTQA | QVMPGD | AVVLSYKSNGFY | SEHNR | FQDSIE | ASNSSGFSGIQPQH |
| 36 | DRGAQA | QVMPGD | AVARSWKSNQFY | SEHNR | FQDSIE | ASSRTGFSGIQPQH |

66. The TCR of claim 61, wherein the TCR is soluble.

67. The TCR of claim 61, wherein the TCR is an αβ heterodimeric TCR;
preferably, the TCR comprises (i) all or part of the TCR α chain other than its transmembrane domain, and (ii) all or part of the TCR β chain other than its transmembrane domain, wherein both of (i) and (ii) comprise the variable domain and at least a portion of the constant domain of the TCR chain;
more preferably, an artificial interchain disulfide bond is contained between the α chain constant region and the β chain constant region of the TCR;
most preferably, cysteine residues forming an artificial interchain disulfide bond between the TCR α chain constant region and β chain constant region are substituted for one or more groups of amino acids selected from the following:
Thr48 of TRAC*01 exon 1 and Ser57 of TRBC1*01 or TRBC2*01 exon 1;
Thr45 of TRAC*01 exon 1 and Ser77 of TRBC1*01 or TRBC2*01 exon 1;
Tyr10 of TRAC*01 exon 1 and Sen 7 of TRBC1*01 or TRBC2*01 exon 1;
Thr45 of TRAC*01 exon 1 and Asp59 of TRBC1*01 or TRBC2*01 exon 1;
Ser15 of TRAC*01 exon 1 and Glu15 of TRBC1*01 or TRBC2*01 exon 1;
Arg53 of TRAC*01 exon 1 and Ser54 of TRBC1*01 or TRBC2*01 exon 1;
Pro89 of TRAC*01 exon 1 and Ala19 of TRBC1*01 or TRBC2*01 exon 1; and
Tyr10 of TRAC*01 exon 1 and Glu20 of TRBC1*01 or TRBC2*01 exon 1.

68. The TCR of claim 61, wherein the amino acid sequence of the α chain variable domain of the TCR is selected from the group consisting of: SEQ ID NOs: 57-82; and/or the amino acid sequence of the β chain variable domain of the TCR is selected from the group consisting of: SEQ ID NOs: and 83-100.

69. The TCR of claim 61, wherein the TCR is selected from the group consisting of:

| | Sequence of α chain variable | Sequence of β chain variable |
|---|---|---|
| TCR No. | domain SEQ ID NO: | domain SEQ ID NO: |
| 1 | 57 | 2 |
| 2 | 58 | 2 |
| 3 | 59 | 2 |
| 4 | 60 | 2 |
| 5 | 61 | 2 |
| 6 | 62 | 2 |
| 7 | 63 | 2 |
| 8 | 1 | 83 |
| 9 | 1 | 84 |
| 10 | 1 | 85 |
| 11 | 1 | 86 |
| 12 | 1 | 87 |
| 13 | 1 | 88 |
| 14 | 1 | 89 |
| 15 | 1 | 90 |
| 16 | 1 | 91 |
| 17 | 1 | 92 |
| 18 | 64 | 93 |
| 19 | 65 | 94 |
| 20 | 66 | 95 |
| 21 | 67 | 96 |
| 22 | 68 | 97 |
| 23 | 69 | 93 |
| 24 | 70 | 95 |
| 25 | 71 | 98 |
| 26 | 72 | 93 |
| 27 | 73 | 93 |
| 28 | 74 | 99 |
| 29 | 75 | 98 |
| 30 | 76 | 98 |
| 31 | 77 | 100 |
| 32 | 78 | 97 |
| 33 | 79 | 98 |
| 34 | 80 | 93 |
| 35 | 81 | 95 |
| 36 | 82 | 97. |

70. The TCR of claim 61, wherein the TCR is a single chain TCR;
preferably, the TCR is a single-chain TCR consisting of an α chain variable domain and a β chain variable domain, and the α chain variable domain and the β chain variable domain are connected by a flexible short peptide sequence (linker).

71. A TCR, wherein the TCR is selected from the group consisting of:

| | Sequence of α chain variable | Sequence of β chain variable |
|---|---|---|
| TCR No. | domain SEQ ID NO: | domain SEQ ID NO: |
| s-1 | 9 | 4 |
| s-2 | 10 | 4 |
| s-3 | 11 | 4 |
| s-4 | 12 | 4 |
| s-5 | 13 | 4 |
| s-6 | 14 | 4 |
| s-7 | 15 | 4 |
| s-8 | 3 | 35 |
| s-9 | 3 | 36 |
| s-10 | 3 | 37 |
| s-11 | 3 | 38 |
| s-12 | 3 | 39 |
| s-13 | 3 | 40 |
| s-14 | 3 | 41 |
| s-15 | 3 | 42 |
| s-16 | 3 | 43 |
| s-17 | 3 | 44 |
| s-18 | 16 | 45 |
| s-19 | 17 | 46 |
| s-20 | 18 | 47 |
| s-21 | 19 | 48 |
| s-22 | 20 | 49 |
| s-23 | 21 | 45 |
| s-24 | 22 | 47 |
| s-25 | 23 | 50 |
| s-26 | 24 | 45 |
| s-27 | 25 | 45 |
| s-28 | 26 | 51 |
| s-29 | 27 | 50 |
| s-30 | 28 | 50 |
| s-31 | 29 | 52 |
| s-32 | 30 | 49 |
| s-33 | 31 | 50 |
| s-34 | 32 | 45 |
| s-35 | 33 | 47 |
| s-36 | 34 | 49. |

72. The TCR of claim 61, wherein a conjugate binds to the α chain and/or β chain of the TCR at C- or N-terminal;
preferably, the conjugate that binds to the TCR is a detectable label, a therapeutic agent, a PK modified moiety, or a combination thereof;
more preferably, wherein the therapeutic agent that binds to the TCR is an anti-CD3 antibody linked to the α or β chain of the TCR at C- or N-terminal.

73. A multivalent TCR complex, wherein the complex comprises at least two TCR molecules, and at least one TCR molecule is the TCR of claim 61.

74. An isolated cell expressing the TCR of claim 61.

75. A pharmaceutical composition, comprising a pharmaceutically acceptable carrier, and a TCR of claim 61, or a TCR complex, or a cell;
wherein the TCR complex comprises at least two TCR molecules, and at least one TCR molecule is the TCR of claim 61; and
the cell expresses the TCR of claim 61.

76. A method for treating a disease, comprising administering an appropriate amount of the TCR of claim 61, or a TCR complex, or a cell, or a pharmaceutical composition to a subject in need thereof;
wherein the TCR complex comprises at least two TCR molecules, and at least one TCR molecule is the TCR of claim 61;
the cell expresses the TCR of claim 61; and
the pharmaceutical composition comprises a pharmaceutically acceptable carrier, and a TCR of claim 61, or a TCR complex comprising at least two TCR molecules, and at least one TCR molecule is the TCR of claim 61, or a cell expressing the TCR of claim 61.

77. A method for treating tumor, comprising administering an appropriate amount of the TCR of claim 61, or a TCR, or a cell to a subject in need thereof;
wherein the TCR complex comprises at least two TCR molecules, and at least one TCR molecule is the TCR of claim 61;
the cell expresses the TCR of claim 61.

78. A method for preparing the T cell receptor of claim 61, comprising the steps of:
(i) culturing a host cell which can express the T cell receptor of claim 61 to express the T cell receptor;
(ii) isolating or purifying the T cell receptor.


SEQUENCE LISTING (145 sequences)

SEQ ID NO: 1 - protein, 112 aa, Artificial sequence
  Synthesized polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVARTYTGNQFYFGTGTSLTVIP

SEQ ID NO: 2 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSQKFSGIQPQHFGDGTRLSIL

SEQ ID NO: 3 - protein, 112 aa, Artificial sequence
  Synthesized polypeptide
QKEVEQNSGPLSVPEGAIVSINCTYSDRGSQSFFWYRQYPGKSPELIMSIYSNGDKEDGRFTAQLNKASQYVSLLIRDVQPSDSATYFCAVARTYTGNQFYFGTGTSLTVIP

SEQ ID NO: 4 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASSSQKFSGIQPQHFGDGTRLSVL

SEQ ID NO: 5 - DNA, 336 bp, Artificial sequence
  Synthesized polynucleotide
caaaaagaagttgaacaaaacagcggtccgctgagcgtgccggagggtgcgatcgttagcattaactgcacctacagcgatcgtggtagccagagcttcttttggtaccgtcaatatccgggcaaaagcccggagctgatcatgagcatttatagcaacggtgacaaggaagatggccgttttaccgcgcagctgaacaaagcgagccaatacgtgagcctgctgatccgtgacgttcagccgagcgatagcgcgacctatttctgcgcggtggcgcgtacctacaccggtaaccagttctattttggtaccggcaccagcctgaccgttattccg

SEQ ID NO: 6 - DNA, 348 bp, Artificial sequence
  Synthesized polynucleotide
gacaccggcgttagccaggatccgcgtcacctgatcgtgaagcgtggtcaaaacgttaccctgcgttgcgacccgattagcgagcacaaccgtctgtactggtatcgtcagaccccgggtcaaggcctggaattcctgacctactttcagaacgaggcgcaactggaaaagagccgtctgctgagcgaccgttttagcgcggaacgtccgaaaggtagcttcagcaccctggagatccagcgtgtggaaccgggcgatagcgcgatgtatttctgcgcgagcagcagccaaaaatttagcggtattcagccgcaacacttcggtgacggcacccgtctgagcgttctg

SEQ ID NO: 7 - protein, 24 aa, Artificial sequence
  Synthesized polypeptide
GGGSEGGGSEGGGSEGGGSEGGTG

SEQ ID NO: 8 - DNA, 72 bp, Artificial sequence
  Synthesized polynucleotide
ggtggcggtagcgagggcggtggcagcgaaggtggcggtagcgagggcggtggcagcgaaggtggcaccggt

SEQ ID NO: 9 - protein, 112 aa, Artificial sequence
  Synthesized polypeptide
QKEVEQNSGPLSVPEGAIVSINCTYSDRGSQSFFWYRQYPGKSPELIMSIYSNGDKEDGRFTAQLNKASQYVSLLIRDVQPSDSATYFCAVARSWKSNQFYFGTGTSLTVIP

SEQ ID NO: 10 - protein, 112 aa, Artificial sequence
  Synthesized polypeptide
QKEVEQNSGPLSVPEGAIVSINCTYSDRGSQSFFWYRQYPGKSPELIMSIYSNGDKEDGRFTAQLNKASQYVSLLIRDVQPSDSATYFCAVARTYLSNRFYFGTGTSLTVIP

SEQ ID NO: 11 - protein, 112 aa, Artificial sequence
  Synthesized polypeptide
QKEVEQNSGPLSVPEGAIVSINCTYSDRGSQSFFWYRQYPGKSPELIMSIYSNGDKEDGRFTAQLNKASQYVSLLIRDVQPSDSATYFCAVARTWASNQFYFGTGTSLTVIP

SEQ ID NO: 12 - protein, 112 aa, Artificial sequence
  Synthesized polypeptide
QKEVEQNSGPLSVPEGAIVSINCTYSDRGSQSFFWYRQYPGKSPELIMSIYSNGDKEDGRFTAQLNKASQYVSLLIRDVQPSDSATYFCAVARSYQSNQFYFGTGTSLTVIP

SEQ ID NO: 13 - protein, 112 aa, Artificial sequence
  Synthesized polypeptide
QKEVEQNSGPLSVPEGAIVSINCTYSDRGSQSFFWYRQYPGKSPELIMSIYSNGDKEDGRFTAQLNKASQYVSLLIRDVQPSDSATYFCAVARSWFSNQFYFGTGTSLTVIP

SEQ ID NO: 14 - protein, 112 aa, Artificial sequence
  Synthesized polypeptide
QKEVEQNSGPLSVPEGAIVSINCTYSDRGSQSFFWYRQYPGKSPELIMSIYSNGDKEDGRFTAQLNKASQYVSLLIRDVQPSDSATYFCAVARTYKSNRFYFGTGTSLTVIP

SEQ ID NO: 15 - protein, 112 aa, Artificial sequence
  Synthesized polypeptide
QKEVEQNSGPLSVPEGAIVSINCTYSDRGSQSFFWYRQYPGKSPELIMSIYSNGDKEDGRFTAQLNKASQYVSLLIRDVQPSDSATYFCAVARTYKSNQFYFGTGTSLTVIP

SEQ ID NO: 16 - protein, 112 aa, Artificial sequence
  Synthesized polypeptide
QKEVEQNSGPLSVPEGAIVSINCTYSDRGSQSFFWYRQYPGKSPELIMSQVMPGDKEDGRFTAQLNKASQYVSLLIRDVQPSDSATYFCAVARSYKSNQFYFGTGTSLTVIP

SEQ ID NO: 17 - protein, 112 aa, Artificial sequence
  Synthesized polypeptide
QKEVEQNSGPLSVPEGAIVSINCTYSDRGSQSFFWYRQYPGKSPELIMSQVVPGDKEDGRFTAQLNKASQYVSLLIRDVQPSDSATYFCAVARTYKSTGFYFGTGTSLTVIP

SEQ ID NO: 18 - protein, 112 aa, Artificial sequence
  Synthesized polypeptide
QKEVEQNSGPLSVPEGAIVSINCTYSDRGTQAFFWYRQYPGKSPELIMSQVMPGDKEDGRFTAQLNKASQYVSLLIRDVQPSDSATYFCAVARTYKSNGFYFGTGTSLTVIP

SEQ ID NO: 19 - protein, 112 aa, Artificial sequence
  Synthesized polypeptide
QKEVEQNSGPLSVPEGAIVSINCTYSDRGSQSFFWYRQYPGKSPELIMSQVMPGDKEDGRFTAQLNKASQYVSLLIRDVQPSDSATYFCAVARSWASNQFYFGTGTSLTVIP

SEQ ID NO: 20 - protein, 112 aa, Artificial sequence
  Synthesized polypeptide
QKEVEQNSGPLSVPEGAIVSINCTYSDRGSQSFFWYRQYPGKSPELIMSQVMPGDKEDGRFTAQLNKASQYVSLLIRDVQPSDSATYFCAVARSWKSNQFYFGTGTSLTVIP

SEQ ID NO: 21 - protein, 112 aa, Artificial sequence
  Synthesized polypeptide
QKEVEQNSGPLSVPEGAIVSINCTYSDRGSQAFFWYRQYPGKSPELIMSQVMPGDKEDGRFTAQLNKASQYVSLLIRDVQPSDSATYFCAVARTYRSTGFYFGTGTSLTVIP

SEQ ID NO: 22 - protein, 112 aa, Artificial sequence
  Synthesized polypeptide
QKEVEQNSGPLSVPEGAIVSINCTYSDRGTQAFFWYRQYPGKSPELIMSQVMPGDKEDGRFTAQLNKASQYVSLLIRDVQPSDSATYFCAVARTYKSTGFYFGTGTSLTVIP

SEQ ID NO: 23 - protein, 112 aa, Artificial sequence
  Synthesized polypeptide
QKEVEQNSGPLSVPEGAIVSINCTYSDRGSQSFFWYRQYPGKSPELIMSQVMPGDKEDGRFTAQLNKASQYVSLLIRDVQPSDSATYFCAVARTWASNQFYFGTGTSLTVIP

SEQ ID NO: 24 - protein, 112 aa, Artificial sequence
  Synthesized polypeptide
QKEVEQNSGPLSVPEGAIVSINCTYSDRGTQAFFWYRQYPGKSPELIMSLVQPGDKEDGRFTAQLNKASQYVSLLIRDVQPSDSATYFCAVARTYKSTGFYFGTGTSLTVIP

SEQ ID NO: 25 - protein, 112 aa, Artificial sequence
  Synthesized polypeptide
QKEVEQNSGPLSVPEGAIVSINCTYSDRGSQAFFWYRQYPGKSPELIMSQVVPGDKEDGRFTAQLNKASQYVSLLIRDVQPSDSATYFCAVARTWASNQFYFGTGTSLTVIP

SEQ ID NO: 26 - protein, 112 aa, Artificial sequence
  Synthesized polypeptide
QKEVEQNSGPLSVPEGAIVSINCTYSDRGSQSFFWYRQYPGKSPELIMSLVQPGDKEDGRFTAQLNKASQYVSLLIRDVQPSDSATYFCAVARTYKSTGFYFGTGTSLTVIP

SEQ ID NO: 27 - protein, 112 aa, Artificial sequence
  Synthesized polypeptide
QKEVEQNSGPLSVPEGAIVSINCTYSDRGAQAFFWYRQYPGKSPELIMSQVMPGDKEDGRFTAQLNKASQYVSLLIRDVQPSDSATYFCAVARSWASNQFYFGTGTSLTVIP

SEQ ID NO: 28 - protein, 112 aa, Artificial sequence
  Synthesized polypeptide
QKEVEQNSGPLSVPEGAIVSINCTYSDRGTQAFFWYRQYPGKSPELIMSQVVPGDKEDGRFTAQLNKASQYVSLLIRDVQPSDSATYFCAVARSWKSNQFYFGTGTSLTVIP

SEQ ID NO: 29 - protein, 112 aa, Artificial sequence
  Synthesized polypeptide
QKEVEQNSGPLSVPEGAIVSINCTYSDRGSQAFFWYRQYPGKSPELIMSQVMPGDKEDGRFTAQLNKASQYVSLLIRDVQPSDSATYFCAVARSWTSNQFYFGTGTSLTVIP

SEQ ID NO: 30 - protein, 112 aa, Artificial sequence
  Synthesized polypeptide
QKEVEQNSGPLSVPEGAIVSINCTYSDRGAQAFFWYRQYPGKSPELIMSIYSNGDKEDGRFTAQLNKASQYVSLLIRDVQPSDSATYFCAVARSYKSNQFYFGTGTSLTVIP

SEQ ID NO: 31 - protein, 112 aa, Artificial sequence
  Synthesized polypeptide
QKEVEQNSGPLSVPEGAIVSINCTYSDRGAQAFFWYRQYPGKSPELIMSTVQDGDKEDGRFTAQLNKASQYVSLLIRDVQPSDSATYFCAVARSWKSNQFYFGTGTSLTVIP

SEQ ID NO: 32 - protein, 112 aa, Artificial sequence
  Synthesized polypeptide
QKEVEQNSGPLSVPEGAIVSINCTYSDRGSQAFFWYRQYPGKSPELIMSIYSNGDKEDGRFTAQLNKASQYVSLLIRDVQPSDSATYFCAVARSWKSNQFYFGTGTSLTVIP

SEQ ID NO: 33 - protein, 112 aa, Artificial sequence
  Synthesized polypeptide
QKEVEQNSGPLSVPEGAIVSINCTYSDRGTQAFFWYRQYPGKSPELIMSQVMPGDKEDGRFTAQLNKASQYVSLLIRDVQPSDSATYFCAVVLSYKSNGFYFGTGTSLTVIP

SEQ ID NO: 34 - protein, 112 aa, Artificial sequence
  Synthesized polypeptide
QKEVEQNSGPLSVPEGAIVSINCTYSDRGAQAFFWYRQYPGKSPELIMSQVMPGDKEDGRFTAQLNKASQYVSLLIRDVQPSDSATYFCAVARSWKSNQFYFGTGTSLTVIP

SEQ ID NO: 35 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASNSSGFSGIQPQHFGDGTRLSVL

SEQ ID NO: 36 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASSASGFSGIQPQHFGDGTRLSVL

SEQ ID NO: 37 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASSPSGFSGIQPQHFGDGTRLSVL

SEQ ID NO: 38 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASNNSGFSGIQPQHFGDGTRLSVL

SEQ ID NO: 39 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASNKSGFSGIQPQHFGDGTRLSVL

SEQ ID NO: 40 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASNSGPVSGIQPQHFGDGTRLSVL

SEQ ID NO: 41 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASNQSGFSGIQPQHFGDGTRLSVL

SEQ ID NO: 42 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASNTSGFSGIQPQHFGDGTRLSVL

SEQ ID NO: 43 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASSMSGFSGIQPQHFGDGTRLSVL

SEQ ID NO: 44 - protein, 116 aa, Artificial sequence
  Synthesized polypeptide
DTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASSSGLLSGIQPQHFGDGTRLSVL

SEQ ID NO: 45 - protein, 116 aa, Artificial sequence
  Synthesized polypeptide
DTGVSQDPRHLIVKRGQNVTLRCDPISEHNRLYWYRQTPGQGLEFLTYFQDSIELEKSRLLSDRFSAERPKGSFSTLEIQRVEPGDSAMYFCASNSGPVSGIQPQHFGDGTRLSVL

SEQ ID NO: 46 - protein, 116 aa, Artificial sequence
  Synthesized polypeptide
DTGVSQDPRHLIVKRGQNVTLRCDPISEHNRLYWYRQTPGQGLEFLTYFQGRSQLEKSRLLSDRFSAERPKGSFSTLEIQRVEPGDSAMYFCASNSGPVSGIQPQHFGDGTRLSVL

SEQ ID NO: 47 - protein, 116 aa, Artificial sequence
  Synthesized polypeptide
DTGVSQDPRHLIVKRGQNVTLRCDPISEHNRLYWYRQTPGQGLEFLTYFQDSIELEKSRLLSDRFSAERPKGSFSTLEIQRVEPGDSAMYFCASNSSGFSGIQPQHFGDGTRLSVL

SEQ ID NO: 48 - protein, 116 aa, Artificial sequence
  Synthesized polypeptide
DTGVSQDPRHLIVKRGQNVTLRCDPISEHNRLYWYRQTPGQGLEFLTYFQDSIELEKSRLLSDRFSAERPKGSFSTLEIQRVEPGDSAMYFCASSMSGFSGIQPQHFGDGTRLSVL

SEQ ID NO: 49 - protein, 116 aa, Artificial sequence
  Synthesized polypeptide
DTGVSQDPRHLIVKRGQNVTLRCDPISEHNRLYWYRQTPGQGLEFLTYFQDSIELEKSRLLSDRFSAERPKGSFSTLEIQRVEPGDSAMYFCASSRTGFSGIQPQHFGDGTRLSVL

SEQ ID NO: 50 - protein, 116 aa, Artificial sequence
  Synthesized polypeptide
DTGVSQDPRHLIVKRGQNVTLRCDPISEHNRLYWYRQTPGQGLEFLTYFQDSIELEKSRLLSDRFSAERPKGSFSTLEIQRVEPGDSAMYFCASNQSGFSGIQPQHFGDGTRLSVL

SEQ ID NO: 51 - protein, 116 aa, Artificial sequence
  Synthesized polypeptide
DTGVSQDPRHLIVKRGQNVTLRCDPISEHNRLYWYRQTPGQGLEFLTYFQGSAQLEKSRLLSDRFSAERPKGSFSTLEIQRVEPGDSAMYFCASNSGPVSGIQPQHFGDGTRLSVL

SEQ ID NO: 52 - protein, 116 aa, Artificial sequence
  Synthesized polypeptide
DTGVSQDPRHLIVKRGQNVTLRCDPISEHNRLYWYRQTPGQGLEFLTYFQDSIELEKSRLLSDRFSAERPKGSFSTLEIQRVEPGDSAMYFCASNQTGFSGIQPQHFGDGTRLSVL

SEQ ID NO: 53 - protein, 252 aa, Artificial sequence
  Synthesized polypeptide
QKEVEQNSGPLSVPEGAIVSINCTYSDRGSQSFFWYRQYPGKSPELIMSIYSNGDKEDGRFTAQLNKASQYVSLLIRDVQPSDSATYFCAVARTYTGNQFYFGTGTSLTVIPGGGSEGGGSEGGGSEGGGSEGGTGDTGVSQDPRHLIVKRGQNVTLRCDPISEHNRLYWYRQTPGQGLEFLTYFQNEAQLEKSRLLSDRFSAERPKGSFSTLEIQRVEPGDSAMYFCASSSQKFSGIQPQHFGDGTRLSVL

SEQ ID NO: 54 - DNA, 756 bp, Artificial sequence
  Synthesized polynucleotide
caaaaagaagttgaacaaaacagcggtccgctgagcgtgccggagggtgcgatcgttagcattaactgcacctacagcgatcgtggtagccagagcttcttttggtaccgtcaatatccgggcaaaagcccggagctgatcatgagcatttatagcaacggtgacaaggaagatggccgttttaccgcgcagctgaacaaagcgagccaatacgtgagcctgctgatccgtgacgttcagccgagcgatagcgcgacctatttctgcgcggtggcgcgtacctacaccggtaaccagttctattttggtaccggcaccagcctgaccgttattccgggtggcggtagcgagggcggtggcagcgaaggtggcggtagcgagggcggtggcagcgaaggtggcaccggtgacaccggcgttagccaggatccgcgtcacctgatcgtgaagcgtggtcaaaacgttaccctgcgttgcgacccgattagcgagcacaaccgtctgtactggtatcgtcagaccccgggtcaaggcctggaattcctgacctactttcagaacgaggcgcaactggaaaagagccgtctgctgagcgaccgttttagcgcggaacgtccgaaaggtagcttcagcaccctggagatccagcgtgtggaaccgggcgatagcgcgatgtatttctgcgcgagcagcagccaaaaatttagcggtattcagccgcaacacttcggtgacggcacccgtctgagcgttctg

SEQ ID NO: 55 - protein, 206 aa, Artificial sequence
  Synthesized polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVARTYTGNQFYFGTGTSLTVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 56 - protein, 246 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSQKFSGIQPQHFGDGTRLSILEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 57 - protein, 112 aa, Artificial sequence
  Synthesized polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVARSWKSNQFYFGTGTSLTVIP

SEQ ID NO: 58 - protein, 112 aa, Artificial sequence
  Synthesized polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVARTYLSNRFYFGTGTSLTVIP

SEQ ID NO: 59 - protein, 112 aa, Artificial sequence
  Synthesized polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVARTWASNQFYFGTGTSLTVIP

SEQ ID NO: 60 - protein, 112 aa, Artificial sequence
  Synthesized polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVARSYQSNQFYFGTGTSLTVIP

SEQ ID NO: 61 - protein, 112 aa, Artificial sequence
  Synthesized polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVARSWFSNQFYFGTGTSLTVIP

SEQ ID NO: 62 - protein, 112 aa, Artificial sequence
  Synthesized polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVARTYKSNRFYFGTGTSLTVIP

SEQ ID NO: 63 - protein, 112 aa, Artificial sequence
  Synthesized polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVARTYKSNQFYFGTGTSLTVIP

SEQ ID NO: 64 - protein, 112 aa, Artificial sequence
  Synthesized polypeptide
QKEVEQNSGPLSVPEGAIASLNCTYSDRGSQSFFWYRQYSGKSPELIMSQVMPGDKEDGRFTAQLNKASQYVSLLIRDSQPSDSATYLCAVARSYKSNQFYFGTGTSLTVIP

SEQ ID NO: 65 - protein, 112 aa, Artificial sequence
  Synthesized polypeptide
QKEVEQNSGPLSVPEGAIASLNCTYSDRGSQSFFWYRQYSGKSPELIMSQVVPGDKEDGRFTAQLNKASQYVSLLIRDSQPSDSATYLCAVARTYKSTGFYFGTGTSLTVIP

SEQ ID NO: 66 - protein, 112 aa, Artificial sequence
  Synthesized polypeptide
QKEVEQNSGPLSVPEGAIASLNCTYSDRGTQAFFWYRQYSGKSPELIMSQVMPGDKEDGRFTAQLNKASQYVSLLIRDSQPSDSATYLCAVARTYKSNGFYFGTGTSLTVIP

SEQ ID NO: 67 - protein, 112 aa, Artificial sequence
  Synthesized polypeptide
QKEVEQNSGPLSVPEGAIASLNCTYSDRGSQSFFWYRQYSGKSPELIMSQVMPGDKEDGRFTAQLNKASQYVSLLIRDSQPSDSATYLCAVARSWASNQFYFGTGTSLTVIP

SEQ ID NO: 68 - protein, 112 aa, Artificial sequence
  Synthesized polypeptide
QKEVEQNSGPLSVPEGAIASLNCTYSDRGSQSFFWYRQYSGKSPELIMSQVMPGDKEDGRFTAQLNKASQYVSLLIRDSQPSDSATYLCAVARSWKSNQFYFGTGTSLTVIP

SEQ ID NO: 69 - protein, 112 aa, Artificial sequence
  Synthesized polypeptide
QKEVEQNSGPLSVPEGAIASLNCTYSDRGSQAFFWYRQYSGKSPELIMSQVMPGDKEDGRFTAQLNKASQYVSLLIRDSQPSDSATYLCAVARTYRSTGFYFGTGTSLTVIP

SEQ ID NO: 70 - protein, 112 aa, Artificial sequence
  Synthesized polypeptide
QKEVEQNSGPLSVPEGAIASLNCTYSDRGTQAFFWYRQYSGKSPELIMSQVMPGDKEDGRFTAQLNKASQYVSLLIRDSQPSDSATYLCAVARTYKSTGFYFGTGTSLTVIP

SEQ ID NO: 71 - protein, 112 aa, Artificial sequence
  Synthesized polypeptide
QKEVEQNSGPLSVPEGAIASLNCTYSDRGSQSFFWYRQYSGKSPELIMSQVMPGDKEDGRFTAQLNKASQYVSLLIRDSQPSDSATYLCAVARTWASNQFYFGTGTSLTVIP

SEQ ID NO: 72 - protein, 112 aa, Artificial sequence
  Synthesized polypeptide
QKEVEQNSGPLSVPEGAIASLNCTYSDRGTQAFFWYRQYSGKSPELIMSLVQPGDKEDGRFTAQLNKASQYVSLLIRDSQPSDSATYLCAVARTYKSTGFYFGTGTSLTVIP

SEQ ID NO: 73 - protein, 112 aa, Artificial sequence
  Synthesized polypeptide
QKEVEQNSGPLSVPEGAIASLNCTYSDRGSQAFFWYRQYSGKSPELIMSQVVPGDKEDGRFTAQLNKASQYVSLLIRDSQPSDSATYLCAVARTWASNQFYFGTGTSLTVIP

SEQ ID NO: 74 - protein, 112 aa, Artificial sequence
  Synthesized polypeptide
QKEVEQNSGPLSVPEGAIASLNCTYSDRGSQSFFWYRQYSGKSPELIMSLVQPGDKEDGRFTAQLNKASQYVSLLIRDSQPSDSATYLCAVARTYKSTGFYFGTGTSLTVIP

SEQ ID NO: 75 - protein, 112 aa, Artificial sequence
  Synthesized polypeptide
QKEVEQNSGPLSVPEGAIASLNCTYSDRGAQAFFWYRQYSGKSPELIMSQVMPGDKEDGRFTAQLNKASQYVSLLIRDSQPSDSATYLCAVARSWASNQFYFGTGTSLTVIP

SEQ ID NO: 76 - protein, 112 aa, Artificial sequence
  Synthesized polypeptide
QKEVEQNSGPLSVPEGAIASLNCTYSDRGTQAFFWYRQYSGKSPELIMSQVVPGDKEDGRFTAQLNKASQYVSLLIRDSQPSDSATYLCAVARSWKSNQFYFGTGTSLTVIP

SEQ ID NO: 77 - protein, 112 aa, Artificial sequence
  Synthesized polypeptide
QKEVEQNSGPLSVPEGAIASLNCTYSDRGSQAFFWYRQYSGKSPELIMSQVMPGDKEDGRFTAQLNKASQYVSLLIRDSQPSDSATYLCAVARSWTSNQFYFGTGTSLTVIP

SEQ ID NO: 78 - protein, 112 aa, Artificial sequence
  Synthesized polypeptide
QKEVEQNSGPLSVPEGAIASLNCTYSDRGAQAFFWYRQYSGKSPELIMSIYSNGDKEDGRFTAQLNKASQYVSLLIRDSQPSDSATYLCAVARSYKSNQFYFGTGTSLTVIP

SEQ ID NO: 79 - protein, 112 aa, Artificial sequence
  Synthesized polypeptide
QKEVEQNSGPLSVPEGAIASLNCTYSDRGAQAFFWYRQYSGKSPELIMSTVQDGDKEDGRFTAQLNKASQYVSLLIRDSQPSDSATYLCAVARSWKSNQFYFGTGTSLTVIP

SEQ ID NO: 80 - protein, 112 aa, Artificial sequence
  Synthesized polypeptide
QKEVEQNSGPLSVPEGAIASLNCTYSDRGSQAFFWYRQYSGKSPELIMSIYSNGDKEDGRFTAQLNKASQYVSLLIRDSQPSDSATYLCAVARSWKSNQFYFGTGTSLTVIP

SEQ ID NO: 81 - protein, 112 aa, Artificial sequence
  Synthesized polypeptide
QKEVEQNSGPLSVPEGAIASLNCTYSDRGTQAFFWYRQYSGKSPELIMSQVMPGDKEDGRFTAQLNKASQYVSLLIRDSQPSDSATYLCAVVLSYKSNGFYFGTGTSLTVIP

SEQ ID NO: 82 - protein, 112 aa, Artificial sequence
  Synthesized polypeptide
QKEVEQNSGPLSVPEGAIASLNCTYSDRGAQAFFWYRQYSGKSPELIMSQVMPGDKEDGRFTAQLNKASQYVSLLIRDSQPSDSATYLCAVARSWKSNQFYFGTGTSLTVIP

SEQ ID NO: 83 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASNSSGFSGIQPQHFGDGTRLSIL

SEQ ID NO: 84 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSASGFSGIQPQHFGDGTRLSIL

SEQ ID NO: 85 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPSGFSGIQPQHFGDGTRLSIL

SEQ ID NO: 86 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASNNSGFSGIQPQHFGDGTRLSIL

SEQ ID NO: 87 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASNKSGFSGIQPQHFGDGTRLSIL

SEQ ID NO: 88 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASNSGPVSGIQPQHFGDGTRLSIL

SEQ ID NO: 89 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASNQSGFSGIQPQHFGDGTRLSIL

SEQ ID NO: 90 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASNTSGFSGIQPQHFGDGTRLSIL

SEQ ID NO: 91 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSMSGFSGIQPQHFGDGTRLSIL

SEQ ID NO: 92 - protein, 116 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSGLLSGIQPQHFGDGTRLSIL

SEQ ID NO: 93 - protein, 116 aa, Artificial sequence
  Synthesized polypeptide
DTGVSQDPRHKITKRGQNVTFRCDPISEHNRLYWYRQTLGQGPEFLTYFQDSIELEKSRLLSDRFSAERPKGSFSTLEIQRTEQGDSAMYLCASNSGPVSGIQPQHFGDGTRLSIL

SEQ ID NO: 94 - protein, 116 aa, Artificial sequence
  Synthesized polypeptide
DTGVSQDPRHKITKRGQNVTFRCDPISEHNRLYWYRQTLGQGPEFLTYFQGRSQLEKSRLLSDRFSAERPKGSFSTLEIQRTEQGDSAMYLCASNSGPVSGIQPQHFGDGTRLSIL

SEQ ID NO: 95 - protein, 116 aa, Artificial sequence
  Synthesized polypeptide
DTGVSQDPRHKITKRGQNVTFRCDPISEHNRLYWYRQTLGQGPEFLTYFQDSIELEKSRLLSDRFSAERPKGSFSTLEIQRTEQGDSAMYLCASNSSGFSGIQPQHFGDGTRLSIL

SEQ ID NO: 96 - protein, 116 aa, Artificial sequence
  Synthesized polypeptide
DTGVSQDPRHKITKRGQNVTFRCDPISEHNRLYWYRQTLGQGPEFLTYFQDSIELEKSRLLSDRFSAERPKGSFSTLEIQRTEQGDSAMYLCASSMSGFSGIQPQHFGDGTRLSIL

SEQ ID NO: 97 - protein, 116 aa, Artificial sequence
  Synthesized polypeptide
DTGVSQDPRHKITKRGQNVTFRCDPISEHNRLYWYRQTLGQGPEFLTYFQDSIELEKSRLLSDRFSAERPKGSFSTLEIQRTEQGDSAMYLCASSRTGFSGIQPQHFGDGTRLSIL

SEQ ID NO: 98 - protein, 116 aa, Artificial sequence
  Synthesized polypeptide
DTGVSQDPRHKITKRGQNVTFRCDPISEHNRLYWYRQTLGQGPEFLTYFQDSIELEKSRLLSDRFSAERPKGSFSTLEIQRTEQGDSAMYLCASNQSGFSGIQPQHFGDGTRLSIL

SEQ ID NO: 99 - protein, 116 aa, Artificial sequence
  Synthesized polypeptide
DTGVSQDPRHKITKRGQNVTFRCDPISEHNRLYWYRQTLGQGPEFLTYFQGSAQLEKSRLLSDRFSAERPKGSFSTLEIQRTEQGDSAMYLCASNSGPVSGIQPQHFGDGTRLSIL

SEQ ID NO: 100 - protein, 116 aa, Artificial sequence
  Synthesized polypeptide
DTGVSQDPRHKITKRGQNVTFRCDPISEHNRLYWYRQTLGQGPEFLTYFQDSIELEKSRLLSDRFSAERPKGSFSTLEIQRTEQGDSAMYLCASNQTGFSGIQPQHFGDGTRLSIL

SEQ ID NO: 101 - protein, 253 aa, Artificial sequence
  Synthesized polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVARTYTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 102 - protein, 293 aa, Artificial sequence
  Synthesized polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSQKFSGIQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 103 - protein, 9 aa, Artificial sequence
  Synthesized polypeptide
VLDGLDVLL

SEQ ID NO: 104 - protein, 6 aa, Artificial Sequence
  Synthesized polypeptide
DRGSQS

SEQ ID NO: 105 - protein, 6 aa, Artificial Sequence
  Synthesized polypeptide
IYSNGD

SEQ ID NO: 106 - protein, 12 aa, Artificial Sequence
  Synthesized polypeptide
AVARTYTGNQFY

SEQ ID NO: 107 - protein, 5 aa, Artificial Sequence
  Synthesized polypeptide
SEHNR

SEQ ID NO: 108 - protein, 6 aa, Artificial Sequence
  Synthesized polypeptide
FQNEAQ

SEQ ID NO: 109 - protein, 14 aa, Artificial Sequence
  Synthesized polypeptide
ASSSQKFSGIQPQH

SEQ ID NO: 110 - protein, 12 aa, Artificial Sequence
  Synthesized polypeptide
AVARSWKSNQFY

SEQ ID NO: 111 - protein, 12 aa, Artificial Sequence
  Synthesized polypeptide
AVARSWASNQFY

SEQ ID NO: 112 - protein, 12 aa, Artificial Sequence
  Synthesized polypeptide
AVARTYRSTGFY

SEQ ID NO: 113 - protein, 12 aa, Artificial Sequence
  Synthesized polypeptide
AVARTYKSTGFY

SEQ ID NO: 114 - protein, 6 aa, Artificial Sequence
  Synthesized polypeptide
DRGTQA

SEQ ID NO: 115 - protein, 6 aa, Artificial Sequence
  Synthesized polypeptide
DRGAQA

SEQ ID NO: 116 - protein, 6 aa, Artificial Sequence
  Synthesized polypeptide
DRGSQA

SEQ ID NO: 117 - protein, 6 aa, Artificial Sequence
  Synthesized polypeptide
QVMPGD

SEQ ID NO: 118 - protein, 6 aa, Artificial Sequence
  Synthesized polypeptide
QVVPGD

SEQ ID NO: 119 - protein, 6 aa, Artificial Sequence
  Synthesized polypeptide
LVQPGD

SEQ ID NO: 120 - protein, 6 aa, Artificial Sequence
  Synthesized polypeptide
FQDSIE

SEQ ID NO: 121 - protein, 6 aa, Artificial Sequence
  Synthesized polypeptide
FQGRSQ

SEQ ID NO: 122 - protein, 14 aa, Artificial Sequence
  Synthesized polypeptide
ASNSGPVSGIQPQH

SEQ ID NO: 123 - protein, 14 aa, Artificial Sequence
  Synthesized polypeptide
ASNQSGFSGIQPQH

SEQ ID NO: 124 - protein, 14 aa, Artificial Sequence
  Synthesized polypeptide
ASSMSGFSGIQPQH

SEQ ID NO: 125 - protein, 14 aa, Artificial Sequence
  Synthesized polypeptide
ASSSGLLSGIQPQH

SEQ ID NO: 126 - protein, 6 aa, Artificial Sequence
  Synthesized polypeptide
TVQDGD

SEQ ID NO: 127 - protein, 12 aa, Artificial Sequence
  Synthesized polypeptide
AVARTYLSNRFY

SEQ ID NO: 128 - protein, 12 aa, Artificial Sequence
  Synthesized polypeptide
AVARTWASNQFY

SEQ ID NO: 129 - protein, 12 aa, Artificial Sequence
  Synthesized polypeptide
AVARSYQSNQFY

SEQ ID NO: 130 - protein, 12 aa, Artificial Sequence
  Synthesized polypeptide
AVARSWFSNQFY

SEQ ID NO: 131 - protein, 12 aa, Artificial Sequence
  Synthesized polypeptide
AVARTYKSNRFY

SEQ ID NO: 132 - protein, 12 aa, Artificial Sequence
  Synthesized polypeptide
AVARTYKSNQFY

SEQ ID NO: 133 - protein, 12 aa, Artificial Sequence
  Synthesized polypeptide
AVARSYKSNQFY

SEQ ID NO: 134 - protein, 12 aa, Artificial Sequence
  Synthesized polypeptide
AVARSWTSNQFY

SEQ ID NO: 135 - protein, 12 aa, Artificial Sequence
  Synthesized polypeptide
AVARTYKSNGFY

SEQ ID NO: 136 - protein, 12 aa, Artificial Sequence
  Synthesized polypeptide
AVVLSYKSNGFY

SEQ ID NO: 137 - protein, 6 aa, Artificial Sequence
  Synthesized polypeptide
FQGSAQ

SEQ ID NO: 138 - protein, 14 aa, Artificial Sequence
  Synthesized polypeptide
ASNSSGFSGIQPQH

SEQ ID NO: 139 - protein, 14 aa, Artificial Sequence
  Synthesized polypeptide
ASSASGFSGIQPQH

SEQ ID NO: 140 - protein, 14 aa, Artificial Sequence
  Synthesized polypeptide
ASSPSGFSGIQPQH

SEQ ID NO: 141 - protein, 14 aa, Artificial Sequence
  Synthesized polypeptide
ASNNSGFSGIQPQH

SEQ ID NO: 142 - protein, 14 aa, Artificial Sequence
  Synthesized polypeptide
ASNKSGFSGIQPQH

SEQ ID NO: 143 - protein, 14 aa, Artificial Sequence
  Synthesized polypeptide
ASNTSGFSGIQPQH

SEQ ID NO: 144 - protein, 14 aa, Artificial Sequence
  Synthesized polypeptide
ASSRTGFSGIQPQH

SEQ ID NO: 145 - protein, 14 aa, Artificial Sequence
  Synthesized polypeptide
ASNQTGFSGIQPQH